(19) United States
(12) Patent Application Publication
(10) Pub. No.: US 2025/0177303 A1
(43) Pub. Date: Jun. 5, 2025

(54) A DRUG NANOCARRIER SYSTEM TO DELIVER A COMBINATION OF TLR AGONISTS AND/OR A LIPOXIN PLUS IMMUNOGENIC CELL DEATH INDUCING CHEMOTHERAPEUTIC AGENTS FOR CANCER IMMUNOTHERAPY

(71) Applicant: The Regents of the University of California, Oakland, CA (US)

(72) Inventors: Andre E. NEL, Sherman Oaks, CA (US); Lijia LUO, Los Angeles, CA (US); Saborni CHATTOPADHYAY, Los Angeles, CA (US)

(73) Assignee: The Regents of the University of California, Oakland, CA (US)

(21) Appl. No.: 18/844,688

(22) PCT Filed: Oct. 19, 2022
(86) PCT No.: PCT/US2022/047177

§ 371 Date: Sep. 6, 2024

Related U.S. Application Data
(60) Provisional application No. 63/318,711, filed on Mar. 10, 2022.
(60) Provisional application No. 63/357,543, filed on Jun. 30, 2022.

(51) Int. Cl. A61K 9/1271; A61K 9/00; A61K 9/51; A61K 31/136; A61K 31/4745; A61K 31/513; A61K 31/519; A61K 31/555; A61K 31/704; A61K 47/69; A61P 35/00

(52) U.S. Cl. CPC A61K 9/1271; A61K 9/0019; A61K 9/5115; A61K 31/136; A61K 31/4745; A61K 31/513; A61K 31/519; A61K 31/555; A61K 31/704; A61K 47/6923; A61P 35/00

(57) ABSTRACT

In various embodiments, drug delivery vehicles are provided for co-delivery of a chemotherapeutic agent and a TLR7/8 agonist and/or a lipoxin to a cancer. In certain embodiments the vehicles comprise a silicasome comprising: a porous nanoparticle encapsulated in a lipid bilayer, where the lipid bilayer contains a lipoxin and/or a lipid compatible TLR7/8 agonist disposed in the lipid bilayer, and the chemotherapeutic agent is contained in pores comprising the porous nanoparticle and the chemotherapeutic agent comprises a chemotherapeutic agent that induces immunogenic cell death (ICD); or a liposome comprising a lipid bilayer where the lipid bilayer contains a lipoxin and/or a lipid compatible a TLR7/8 agonist disposed in the lipid bilayer; and the chemotherapeutic agent is inside the liposome and the chemotherapeutic agent comprises a chemotherapeutic agent that induces immunogenic cell death (ICD).

### CROSS-REFERENCE TO RELATED APPLICATIONS

This application claims priority to and benefit of U.S. Ser. No. 63/357,543, filed on Jun. 30, 2022 and U.S. Ser. No. 63/318,711, filed on Mar. 10, 2022, both of which are incorporated herein by reference in their entirety for all purposes.

### STATEMENT OF GOVERNMENTAL SUPPORT

This invention was made with government support under Grant No. CA247666, awarded by the National Institutes of Health. The government has certain rights in the invention.

### INCORPORATION BY REFERENCE OF SEQUENCE LISTING PROVIDED AS A TEXT FILE

[Not Applicable]

### BACKGROUND

Pancreatic ductal adenocarcinomas (PDAC) has the worst prognosis of solid cancers, with a 5-year survival rate of approximately 8%. This high rate of mortality is due to several factors, including late presentation, aggressive biology with early metastatic spread, presence of dysplastic stroma that interferes in drug delivery, also promoting drug resistance. In addition, the dysplastic stroma is responsible for immune suppressive effects, which are further enhanced by a low burden of new neoantigens and tumor immunogenicity. Finally, PDAC exhibits resistance to many antineoplastic therapies, with rapid progression and low rates of a pathologic complete response. According to the American Cancer Society, approximately 60430 new cases of pancreatic cancer were diagnosed in the USA in 2021, leading to mortality in 48220 cases.

Irinotecan, a topoisomerase I inhibitor, is frequently used for PDAC chemotherapy, and can exert a significant impact on survival improvement when used in combination with other drugs in a combination known as the FOLFIRINOX (folinic acid, 5-fluorouracil, irinotecan and oxaliplatin) regimen. More recently, Irinotecan has also been available in the form of a liposome (ONIVYDE®), which can improve the efficacy of delivery and lowering of drug toxicity. However, liposomes are leaky and has resulted in ONIVYDE® receiving a black box warning from the FDA. For that reason, we have developed a lipid bilayer coated mesoporous silica nanoparticle (MSNP) platform, also known as a silicasome for improving drug delivery to PDAC and other cancer sites (see, e.g., Liu et al. (2016) ACS Nano, 10(2): 2702-2715 DOI: 10.1021/acsnano.5b07781). This nanocarrier holds several advantages over ONIVYDE® from the perspective of allowing improved drug loading, exhibiting less leakiness, improving drug delivery and reducing systemic toxicity (particularly in the bone marrow, gastrointestinal tract and the liver). We have also recently demonstrated that the Irinotecan-silicasome carrier is capable of generating immunogenic cell death, which triggers an anti-PDAC immune response that can be further propagated by anti-PD1 monoclonal antibodies (see, e.g., Liu et al. (2021) Adv. Sci. 8(6): 2002147).

The PDAC tumor microenvironment is comprised of a complex cellular network, which, in addition to the ductal cancer cells, includes cancer-associated fibroblasts, myeloid suppressor cells, M2 macrophages, and regulatory T-cells endothelial cells. Many of these cells exert immune suppressive effects, which are further accentuated by the dysplastic stroma, which plays a role in recruiting these immune suppressive cell types to the cancer site. This helps to establish a complex immune landscape, in which a large number of immune escape mechanisms can interfere in the activity of cytotoxic CD8+ T-cells.

While from an immunotherapeutic perspective it is possible to improve the rigor of the ICD-induced immune response by combination therapy with immune checkpoint inhibitors (such as anti-PD1, this only leads to a partial improvement in the response outcome, necessitating the use of additional immune modulatory agents to reprogram the tumor microenvironment. Toll-like receptors (TLRs) are widely expressed by innate and cognitive cellular elements in the PDAC tumor microenvironment, with the possibility to augment the immune response at the tumor site, either independently or in combination with chemotherapeutic agents that induce immunogenic cell death. Among the different TLRs, the endosomal TLR7 and TLR8 complexes have been of particular interest since both are expressed by all the major human dendritic cell (DC) subsets, as well as by human B cells. Thus, the triggering of signal transduction through engagement of TLRs (including TLR7), can lead to the activation of antigen-presenting cells (APCs) and the introduction of pro-inflammatory responses in which cytokines and chemokines that can augment the anti-tumor immune response. This includes improved antigen presentation, as well as the ability to reprogram the immune suppressive effects of myeloid derived dendritic cells and M2 macrophages.

While small molecule and synthetic lipids can be used therapeutically as TLR7 agonists, there is the potential downside of systemic toxicity, including the generation of a cytokine storm. Therefore, the development of injectable, local-release formulations of TLR7/8 agonists with physicochemical properties is an area of intense study and drug development.

### SUMMARY

Various embodiments provided herein may include, but need not be limited to, one or more of the following:

Embodiment 1: A drug delivery vehicle for the co-delivery of a chemotherapeutic agent and a Toll-Like Receptor (TLR) agonist and/or a lipoxin, said vehicle comprising:
a silicasome comprising:
   a porous nanoparticle encapsulated in a lipid bilayer, where:
   said lipid bilayer contains a lipoxin and/or a lipid compatible Toll-Like Receptor (TLR) agonist disposed in the lipid bilayer; and
   said chemotherapeutic agent is contained in pores comprising said porous nanoparticle and said chemotherapeutic agent comprises a chemotherapeutic agent that induces immunogenic cell death (ICD); or
a liposome comprising a lipid bilayer where:
   said lipid bilayer contains a lipoxin and/or a lipid compatible a Toll-Like Receptor (TLR) agonist disposed in the lipid bilayer; and
   said chemotherapeutic agent is inside said liposome and said chemotherapeutic agent comprises a chemotherapeutic agent that induces immunogenic cell death (ICD).

Embodiment 2: The drug delivery vehicle of embodiment 1, wherein said TLR agonist comprises a TLR7/8 agonist.

Embodiment 3: The drug delivery vehicle according to any one of embodiments 1-2, wherein said vehicle comprises a silicasome.

Embodiment 4: The drug delivery vehicle of embodiment 3, wherein said porous nanoparticle comprises a mesoporous silica nanoparticle.

Embodiment 5: The drug delivery vehicle according to any one of embodiments 1-2, wherein said vehicle comprises a liposome.

Embodiment 6: The drug delivery vehicle according to any one of embodiments 1-5, wherein said drug delivery vehicle comprises as lipid compatible TLR agonist.

Embodiment 7: The drug delivery vehicle of embodiment 6, wherein said drug delivery vehicle comprises a lipidated TLR agonist.

Embodiment 8: The drug delivery vehicle according to any one of embodiments 6-7, wherein said drug delivery vehicle comprises a TLR7/TLR8 agonist.

Embodiment 9: The drug delivery vehicle of embodiment 8, wherein said drug delivery vehicle comprises a lipidated TLR7/8 agonist selected from the group consisting of 3M-052, lipidated UM-3001, and an imidazoquinoline molecule covalently linked to a phospho- or phosphonolipid group.

Embodiment 10: The drug delivery vehicle of embodiment 9, wherein said lipidated TLR7/8 agonist comprises 3M-052 (Telratolimod).

Embodiment 11: The drug delivery vehicle of embodiment 9, wherein said lipidated TLR7/8 agonist comprises a lipidated UM-3001 selected from the group consisting of UM-3003, UM-3004, and UM-3005.

Embodiment 12: The drug delivery vehicle of embodiment 11, wherein said lipidated imidazoquinoline comprises UM-3003.

Embodiment 13: The drug delivery vehicle of embodiment 11, wherein said lipidated imidazoquinoline comprises UM-3004.

Embodiment 14: The drug delivery vehicle of embodiment 11, wherein said lipidated imidazoquinoline comprises UM-3005.

Embodiment 15: The drug delivery vehicle of embodiment 9, wherein said lipidated TLR7/8 agonist comprises a lipidated imidazoquinoline a molecule selected from the group consisting of L1, L2, L3, L4, and L5.

Embodiment 16: The drug delivery vehicle of embodiment 15, wherein said lipidated imidazoquinoline comprises L1.

Embodiment 17: The drug delivery vehicle of embodiment 15, wherein said lipidated imidazoquinoline comprises L2.

Embodiment 18: The drug delivery vehicle of embodiment 15, wherein said lipidated imidazoquinoline comprises L3.

Embodiment 19: The drug delivery vehicle of embodiment 15, wherein said lipidated imidazoquinoline comprises L4.

Embodiment 20: The drug delivery vehicle of embodiment 15, wherein said lipidated imidazoquinoline comprises L5.

Embodiment 21: The drug delivery vehicle according to any one of embodiments 1-20, wherein said drug delivery vehicle comprises a lipoxin.

Embodiment 22: The drug delivery vehicle of embodiment 21, wherein said lipoxin comprises LXA4 or an analog thereof.

Embodiment 23: The drug delivery vehicle of embodiment 22, wherein said lipoxin comprises a lipoxin selected from the group consisting of 16-phenoxy-LXA4-Me, 15-cyclohexyl-LXA4-Me, and 15-R/S-methyl-LXA4-Me.

Embodiment 24: The drug delivery vehicle according to any one of embodiments 6-7, wherein said drug delivery vehicle comprises a TLR agonist shown in Table 1, and/or shown in FIG. 33, and/or selected from the group consisting of MEDI9197, 3M-052 (Telratolimod), MPLA (PHAD®), KRN7000, Kdo2-Lipid A ammonium, Pam2CSK4, Pam3CSK4, FSL-1, CRX-527, LXA4, Resolvins (D series 1-6), and Resolvins (E series 1-6).

Embodiment 25: The drug delivery vehicle according to any one of embodiments 1-24, wherein said chemotherapeutic agent is an ICD inducer selected from the group consisting of mitoxantrone (MTX), doxorubicin (DOX), oxaliplatin, anthracenedione, bleomycin, bortezomib, cisplatin, daunorubicin, docetaxel, epirubicin, idarubicin, paclitaxel, R2016, cyclophosphamide, and irinotecan.

Embodiment 26: The drug delivery vehicle of embodiment 25, wherein said chemotherapeutic agent comprises irinotecan (IRIN).

Embodiment 27: The drug delivery vehicle of embodiment 25, wherein said chemotherapeutic agent comprises mitoxantrone (MTX).

Embodiment 28: The drug delivery vehicle of embodiment 25, wherein said chemotherapeutic agent comprises oxaliplatin.

Embodiment 29: The drug delivery vehicle of embodiment 25, wherein said chemotherapeutic agent comprises doxorubicin.

Embodiment 30: The drug delivery vehicle of according to any one of embodiments 1-29, wherein said lipid bilayer comprises a phospholipid.

Embodiment 31: The drug delivery vehicle of embodiment 30, wherein said phospholipid comprises a saturated fatty acid with a C14-C20 carbon chain, and/or an unsaturated fatty acid with a C14-C20 carbon chain, and/or a natural lipid comprising a mixture of fatty acids with C12-C20 carbon chains.

Embodiment 32: The drug delivery vehicle of embodiment 31, wherein said phospholipid comprises a phospholipid selected from the group consisting of phosphatidylcholine (DPPC), 1,2-dimyristoleoyl-sn-glycero-3-phosphocholine (DMPC), 1,2-dioleoyl-sn-glycero-3-phosphocholine (DOPC), 1,2-distearoyl-sn-glycero-3-phospho-rac-glycerol (DSPG), 1,2-dipalmitoyl-sn-glycero-3-phosphoglycerol (DPPG), distearoylphosphatidylcholine (DSPC), 1,2-dieicosenoyl-sn-glycero-3-phosphocholine, and diactylphosphatidylcholine (DAPC).

Embodiment 33: The drug delivery vehicle of embodiment 31, wherein said phospholipid comprises a natural lipid selected from the group consisting of egg phosphatidylcholine (egg PC), and soy phosphatidylcholine (soy PC).

Embodiment 34: The drug delivery vehicle of embodiment 31, wherein said phospholipid comprises distearoylphosphatidylcholine (DSPC).

Embodiment 35: The drug delivery vehicle according to any one of embodiments 30-34, wherein said lipid bilayer comprises an mPEG phospholipid with a phospholipid C14-C18 carbon chain, and a PEG molecular weight ranging from about 350 Da to 5000 Da.

Embodiment 36: The drug delivery vehicle of embodiment 35, wherein said lipid bilayer comprises 1,2-distearoyl-sn-glycero-3-phosphoethanolamine-PEG (DSPE-PEG).

Embodiment 37: The drug delivery vehicle of embodiment 36, wherein said DSPE-PEG comprises DPSE-PEG_{2K} or DPSE-PEG_{5K}.

Embodiment 38: The drug delivery vehicle according to any one of embodiments 34-37, wherein said lipid bilayer comprises DSPC:CHOL and/or CHEMS DSPE-PEG: lipid compatible TLR7/TLR8 agonist and/or lipoxin.

Embodiment 39: The drug delivery vehicle of embodiment 38, wherein the ratio of DSPC:CHOL and/or CHEMS:DSPE-PEG:TLR7/8 agonist ranges from 40-90% DSPC:10%-50% CHEL and/or CHEMS: 1%-10% DSPE-PEG:1%-20% TLR7/8 agonist (molar ratio).

Embodiment 40: The drug delivery vehicle of embodiment 39, wherein the lipid bilayer comprise 55.5:38.5:2.7:3.3 for DSPC, cholesterol, DSPE-PEG₂ₖ and TLR7/8 agonist.

Embodiment 41: The drug delivery vehicle of embodiment 40, wherein said TLR7/8 agonist comprises 3M-052.

Embodiment 42: The drug delivery vehicle of embodiment 38, wherein the ratio of DSPC:CHOL and/or CHEMS:DSPE-PEG:TLR7/8 agonist ranges from 40-90% DSPC:10%-50% CHEL and/or CHEMS: 1%-10% DSPE-PEG:0.1%-20% lipoxin (molar ratio).

Embodiment 43: The drug delivery vehicle of embodiment 42, wherein the lipid bilayer comprise 55.4:39.6:4.7:0.2 for DSPC, cholesterol, DSPE-PEG₂ₖ and lipoxin.

Embodiment 44: The drug delivery vehicle of embodiment 43, wherein said lipoxin comprises LXA4.

Embodiment 45: The drug delivery vehicle according to any one of embodiments 30-44, wherein said lipid bilayer comprises a cholesterol derivative selected from the group consisting of cholesterol hemisuccinate (CHEMS), lysine-based cholesterol (CHLYS), and PEGylated cholesterol (Chol-PEG).

Embodiment 46: The drug delivery vehicle of embodiment 45, wherein said lipid bilayer comprises CHEMS.

Embodiment 47: The drug delivery vehicle of embodiment 46, wherein said bilayer comprises CHEMS ranging from about 5% (mol percent) up to about 30% total lipid.

Embodiment 48: The drug delivery vehicle of embodiment 47, wherein said bilayer comprise about 10% or about 20% CHEMS or about 30% CHEMS or about 40% CHEMS.

Embodiment 49: The drug delivery vehicle according to any one of embodiments 1-48, wherein when the drug delivery vehicle contains a cargo-trapping agent (e.g., protonating agent).

Embodiment 50: The drug delivery vehicle of embodiment 49, wherein said cargo trapping agent before reaction with the chemotherapeutic agent loaded in drug delivery vehicle is selected from the group consisting of citric acid, triethylammonium sucrose octasulfate (TEA₈SOS), (NH₄)₂SO₄, an ammonium salt, a trimethylammonium salt, and a triethylammonium salt.

Embodiment 51: The drug delivery vehicle of embodiment 50, wherein said cargo-trapping agent before reaction with said chemotherapeutic agent is citric acid.

Embodiment 52: The drug delivery vehicle of embodiment 50, wherein said cargo-trapping agent before reaction with said chemotherapeutic agent is ammonium sulfate.

Embodiment 53: The drug delivery vehicle according to any one of embodiments 1-52, wherein said drug carrier is conjugated to a moiety selected from the group consisting of a targeting moiety, a fusogenic peptide, and a transport peptide.

Embodiment 54: The drug delivery vehicle according to any one of embodiments 1-53, wherein:
said drug delivery vehicle in suspension is stable for at least 1 month, or at least 2 months, or at least 3 months, or at least 4 months, or at least 5 months, or at least 6 months when stored at 4° C.; and/or
said drug delivery vehicle forms a stable suspension on rehydration after lyophilization; and/or
said drug delivery vehicle shows reduced drug toxicity as compared to free drug; and/or
said drug delivery vehicle colloidal stability in physiological fluids with pH 7.4 and remains monodisperse to allow systemic biodistribution and is capable of entering a disease site by vascular leakage (EPR effect) or transcytosis.

Embodiment 55: The drug delivery vehicle drug carrier according to any one of embodiments 1-54, wherein said carrier is colloidally stable.

Embodiment 56: A pharmaceutical formulation comprising:
a drug delivery vehicle according to any one of embodiments 1-55; and
a pharmaceutically acceptable carrier.

Embodiment 57: The pharmaceutical formulation of embodiment 56, wherein said formulation is formulated for administration via a route selected from the group consisting of intravenous administration, intraarterial administration, intracerebral administration, intrathecal administration, oral administration, aerosol administration, administration via inhalation, intracranial administration via a cannula, and subcutaneous or intramuscular depot deposition.

Embodiment 58: The pharmaceutical formulation according to any one of embodiments 56-57, wherein said formulation is formulated for systemic administration.

Embodiment 59: The pharmaceutical formulation according to any one of embodiments 56-58, wherein said formulation is sterile.

Embodiment 60: The pharmaceutical formulation according to any one of embodiments 56-59, wherein said formulation is a unit dosage formulation.

Embodiment 61: A method of treating a cancer in a mammal, said method comprising administering to said mammal an effective amount of a drug delivery carrier according to any one of embodiments 1-55.

Embodiment 62: The method of embodiment 61, wherein said administering comprises administering an effective amount of a pharmaceutical formation according to any one of embodiments 56-61.

Embodiment 63: The method according to any one of embodiments 61-62, wherein said cancer comprises a cancer selected from the group consisting of pancreatic ductal adenocarcinoma (PDAC). In certain embodiments the cancer is a cancer selected from the group consisting of acute lymphoblastic leukemia (ALL), acute myeloid leukemia (AML), adrenocortical carcinoma, AIDS-related cancers (e.g., Kaposi sarcoma, lymphoma), anal cancer, appendix cancer, astrocytomas, atypical teratoid/rhabdoid tumor, bile duct cancer, extrahepatic cancer, bladder cancer, bone cancer (e.g., Ewing sarcoma, osteosarcoma, malignant fibrous histiocytoma), brain stem glioma, brain tumors (e.g., astrocytomas, glioblastoma, brain and spinal cord tumors, brain stem glioma, central nervous system atypical teratoid/rhabdoid tumor, central nervous system embryonal tumors, central nervous system germ cell tumors, craniopharyngioma, ependymoma, breast cancer, bronchial tumors, burkitt lymphoma, carcinoid tumors (e.g., childhood, gastrointestinal), cardiac tumors, cervical cancer, chordoma, chronic lymphocytic leukemia (CLL), chronic myelogenous leukemia (CML), chronic myeloproliferative disorders, colon cancer, colorectal cancer, craniopharyngioma, cutaneous t-cell lymphoma, duct cancers e.g. (bile, extrahepatic), ductal carcinoma in situ (DCIS), embryonal tumors, endometrial cancer, ependymoma, esophageal cancer, esthesioneuroblastoma, extracranial germ cell tumor, extragonadal germ cell tumor, extrahepatic bile duct cancer, eye cancer (e.g., intraocular melanoma, retinoblastoma), fibrous histiocytoma of bone, malignant, and osteosarcoma, gallbladder cancer, gastric (stomach) cancer, gastrointestinal carcinoid tumor, gastrointestinal stromal tumors (GIST), germ cell tumors (e.g., ovarian cancer, testicular cancer, extracranial cancers, extragonadal cancers, central nervous system), gestational trophoblastic tumor, brain stem cancer, hairy cell leukemia, head and neck cancer, heart cancer, hepatocellular (liver) cancer, histiocytosis, langerhans cell cancer, Hodgkin lymphoma, hypopharyngeal cancer, intraocular melanoma, islet cell tumors, pancreatic neuroendocrine tumors, kaposi sarcoma, kidney cancer (e.g., renal cell, Wilm's tumor, and other kidney tumors), langerhans cell histiocytosis, laryngeal cancer, leukemia, acute lymphoblastic (ALL), acute myeloid (AML), chronic lymphocytic (CLL), chronic myelogenous (CML), hairy cell, lip and oral cavity cancer, liver cancer (primary), lobular carcinoma in situ (LCIS), lung cancer (e.g., childhood, non-small cell, small cell), lymphoma (e.g., AIDS-related, Burkitt (e.g., non-Hodgkin lymphoma), cutaneous T-Cell (e.g., mycosis fungoides, Sézary syndrome), Hodgkin, non-Hodgkin, primary central nervous system (CNS)), macroglobulinemia, Waldenström, male breast cancer, malignant fibrous histiocytoma of bone and osteosarcoma, melanoma (e.g., childhood, intraocular (eye)), merkel cell carcinoma, mesothelioma, metastatic squamous neck cancer, midline tract carcinoma, mouth cancer, multiple endocrine neoplasia syndromes, multiple myeloma/plasma cell neoplasm, mycosis fungoides, myelodysplastic syndromes, chronic myeloid leukemia (CML), multiple myeloma, nasal cavity and paranasal sinus cancer, nasopharyngeal cancer, neuroblastoma, oral cavity cancer, lip and oropharyngeal cancer, osteosarcoma, ovarian cancer, pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, plasma cell neoplasm, pleuropulmonary blastoma, primary central nervous system (CNS) lymphoma, prostate cancer, rectal cancer, renal cell (kidney) cancer, renal pelvis and ureter, transitional cell cancer, rhabdomyosarcoma, salivary gland cancer, sarcoma (e.g., Ewing, Kaposi, osteosarcoma, rhadomyosarcoma, soft tissue, uterine), Sézary syndrome, skin cancer (e.g., melanoma, merkel cell carcinoma, basal cell carcinoma, nonmelanoma), small intestine cancer, squamous cell carcinoma, squamous neck cancer with occult primary, stomach (gastric) cancer, testicular cancer, throat cancer, thymoma and thymic carcinoma, thyroid cancer, trophoblastic tumor, ureter and renal pelvis cancer, urethral cancer, uterine cancer, endometrial cancer, uterine sarcoma, vaginal cancer, vulvar cancer, Waldenström macroglobulinemia, and Wilm's tumor.

Embodiment 64: The method of embodiment 63, wherein said cancer comprise pancreatic cancer.

Embodiment 65: The method of embodiment 64, wherein said cancer comprises advanced PDAC.

Embodiment 66: The method according to any one of embodiments 64-65, wherein said drug delivery carrier comprises a component in a drug combination known as the FOLFIRINOX (folinic acid, 5-fluorouracil, irinotecan, and oxaliplatin) regimen.

Embodiment 67: The method of embodiment 66, wherein said drug delivery carrier comprises irinotecan and said carrier replaces irinotecan in the FOLFIRINOX drug combination.

Embodiment 68: The method of embodiment 66, wherein said drug delivery carrier comprises oxaliplatin and said carrier replaces oxaliplatin in the FOLFIRINOX drug combination.

Embodiment 69: The method of embodiment 66, wherein said drug delivery carrier is administered in addition to the combination of folinic acid, 5-fluorouracil, irinotecan, and oxaliplatin.

### Definitions

The terms “subject,” “individual,” and “patient” may be used interchangeably and refer to humans, as well as non-human mammals (e.g., non-human primates, canines, equines, felines, porcines, bovines, ungulates, lagomorphs, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

As used herein, the phrase “a subject in need thereof” refers to a subject, as described infra, that suffers from, or is at risk for a cancer as described herein. Thus, for example, in certain embodiments the subject is a subject with a cancer (e.g., pancreatic ductal adenocarcinoma (PDAC), breast cancer (e.g., drug-resistant breast cancer), colon cancer, brain cancer, and the like). In certain embodiments the methods described herein are prophylactic and the subject is one in whom a cancer is to be inhibited or prevented. In certain embodiments the subject for prophylaxis is one with a family history of cancer and/or a risk factor for a cancer (e.g., a genetic risk factor, an environmental exposure, and the like).

The term “treat” when used with reference to treating, e.g., a pathology or disease refers to the mitigation and/or elimination of one or more symptoms of that pathology or disease, and/or a delay in the progression and/or a reduction in the rate of onset or severity of one or more symptoms of that pathology or disease, and/or the prevention of that pathology or disease. The term treat can refer to prophylactic treatment which includes a delay in the onset or the prevention of the onset of a pathology or disease.

The terms “coadministration” or “administration in conjunction with” or “cotreatment” when used in reference to the coadministration of a first compound (or component) (e.g., an ICD inducer) and a second compound (or component) (e.g., an IDO inhibitor) indicates that the first compound (or component) and the second compound (or component) are administered so that there is at least some chronological overlap in the biological activity of first compound and the second compound in the organism to which they are administered. Coadministration can include simultaneous administration or sequential administration. In sequential administration there may even be some substantial delay (e.g., minutes or even hours) between administration of the first compound and the second compound as long as their biological activities overlap. In certain embodiments, the coadminstration is over a time frame that permits the first compound and second compound to produce an enhanced therapeutic or prophylactic effect on the organism. In certain embodiments the enhanced effect is a synergistic effect.

The term “immunogenic cell death” or “ICD” refers to a unique form of cell death caused by some cytostatic agents such as anthracyclines (Obeid et al. (2007) Nature Med., 13(1): 54-61), anthracenedione (mitoxantrone, aka MTX), oxaliplatin, irinotecan, and bortezomib, or radiotherapy and/or photodynamic therapy (PDT). Unlike regular apoptosis, which is mostly non-immunogenic or even tolerogenic, immunogenic apoptosis of cancer cells can induce an effective antitumor immune response through activation of dendritic cells (DCs) and consequent activation of specific T cell responses (Spisek and Dhodapkar (2007) Cell Cycle, 6(16): 1962-1965). Endoplasmic reticulum (ER) stress, reactive oxygen species (ROS) production and induction of autophagy are key intracellular response pathways that govern ICD (Krysko et al. (2012) Nat. Rev. Canc. 12(12): 860-875). In addition to facilitating tumor cell death that facilitates antigen presentation by dendritic cells, ICD is characterized by secretion or release of damage-associated molecular patterns (DAMPs), which exert additional immune adjuvant effects. Calreticulin (CRT), one of the DAMP molecules, which is normally in the lumen of the ER, is translocated to the surface of dying cell where it functions as an “eat me” signal for phagocytes. Other important surface exposed DAMPs are heat-shock proteins (HSPs), namely HSP70 and HSP90, which under stress condition are also translocated to the plasma membrane. On the cell surface they have an immunostimulatory effect, based on their interaction with number of antigen-presenting cell (APC) surface receptors like CD91 and CD40 and also facilitate cross-presentation of antigens derived from tumor cells on MHC class I molecule, which then triggers CD8+ T cell activation and expansion. Other important DAMPs, characteristic for ICD are secreted amphoterin (HMGB1) and ATP (see, e.g., Apetoh et al. (2007) Nature Med. 13(9): 1050-1059; Ghiringhelli et al. (2009) Nature Med. 15(10): 1170-1178). HMGB1 is considered to be a late apoptotic marker and its release to the extracellular space appears to be required for the optimal release and presentation of tumor antigens to dendritic cells. It binds to several pattern recognition receptors (PRRs) such as Toll-like receptor (TLR) 2 and 4, which are expressed on APCs. The most recently found DAMP released during immunogenic cell death is ATP, which functions as a “find-me” signal for monocytes when secreted and induces their attraction to the site of apoptosis (see, e.g., Garg et al. (2012) EMBO J. 31(5): 1062-1079). ATP binds to purinergic receptors on APCs.

The terms “nanocarrier” and “nanoparticle drug carrier” and drug delivery vehicle are used interchangeably and refer to a nanostructure an interior core region into which one or more drugs can be disposed. In certain embodiments the nanocarrier comprises a lipid bilayer encasing (or surrounding or enveloping) an aqueous core and thereby forms a liposome. In certain embodiments the nanocarrier comprises a lipid bilayer encasing (or surrounding a porous nanoparticle core and thereby forms a silicasome.

As used herein, the term “lipid” refers to conventional lipids, phospholipids, cholesterol, cholesterol hemisuccinate, and chemically functionalized lipids for attachment of PEG and ligands, etc.

As used herein, the terms “lipid bilayer” or “LB” refers to any double layer of oriented amphipathic lipid molecules in which the hydrocarbon tails face inward to form a continuous non-polar phase.

As used herein, the terms “liposome” or “lipid vesicle” or “vesicle” are used interchangeably to refer to an aqueous compartment enclosed by a lipid bilayer, as being conventionally defined (see, e.g., Stryer (1981) Biochemistry, 2d Edition, W. H. Freeman & Co., p. 213). In certain embodiments the drugs and/or targeting moieties are covalently coupled (e.g., directly or through a linker) to the lipid bilayer. In certain embodiments compatible moieties are lipid compatible/lipid soluble moieties (e.g., drugs) that are disposed within the lipid bilayer.

The term “silicasome” refers to a porous nanoparticle encapsulated in a lipid bilayer. In certain embodiments the porous nanoparticle comprises a mesoporous silica nanoparticle. In certain embodiments the silicasome contains a drug disposed in the interior of the nanoparticle (e.g., in the pores comprising the nanoparticle) and/or drugs and/or targeting moieties associated with the lipid bilayer. In certain embodiments the drugs and/or targeting moieties are covalently coupled (e.g., directly or through a linker) to the lipid bilayer. In certain embodiments compatible moieties are lipid compatible/lipid soluble moieties (e.g., drugs) that are disposed within the lipid bilayer.

The term “liposome” refers to an artificial vesicle consisting of an aqueous core enclosed in a lipid bilayer, e.g., a lipid bilayer comprising phospholipids and/or other lipid molecules. In certain embodiments the liposome contains a drug in the aqueous interior and/or drugs and/or targeting moieties associated with the lipid bilayer (lipid membrane). In certain embodiments the drugs and/or targeting moieties are covalently coupled (e.g., directly or through a linker) to the lipid bilayer. In certain embodiments compatible moieties are (e.g., lipid compatible/lipid soluble) moieties (e.g., drugs) are disposed within the lipid bilayer.

Compared with the lipid bilayer coated on a porous nanoparticle (e.g., a mesoporous silica nanoparticle, the lipid bilayer in a lipid vesicle or liposome can be referred to as an “unsupported lipid bilayer” and the lipid vesicle itself (when unloaded) can be referred to as an “empty vesicle”. The lipid bilayer coated on a nanoparticle (e.g., a mesoporous silica nanoparticle) can be referred to as a “supported lipid bilayer” because the lipid bilayer is located on the surface and supported by a porous particle core. In certain embodiments, the lipid bilayer can have a thickness ranging from about 6 nm to about 7 nm which includes a 3-4 nm thickness of the hydrophobic core, plus the hydrated hydrophilic head group layers (each about 0.9 nm) plus two partially hydrated regions of about 0.3 nm each. In various embodiments, the lipid bilayer surrounding the nanoparticle comprises a continuous bilayer or substantially continuous bilayer that effectively encapsulates and seals the nanoparticle.

As used herein, the term “selective targeting” or “specific binding” refers to use of targeting ligands on the surface of a drug delivery nanocarrier (e.g., a LB-coated nanoparticle). In certain embodiments the targeting ligand(s) are on the surface of a lipid bilayer or LB-coated nanoparticle. Typically, the ligands interact specifically/selectively with receptors or other biomolecular components expressed on the target, e.g., a cell surface of interest. The targeting ligands can include such molecules and/or materials as peptides, antibodies, aptamers, targeting peptides, polysaccharides, and the like.

A drug delivery vehicle having targeting ligands can be referred to as a “targeted drug delivery vehicle”.

The term “about” or “approximately” as used herein refers to being within an acceptable error range for the particular value as determined by one of ordinary skill in the art, which will depend in part on how the value is measured or determined, i.e. the limitations of the measurement system, i.e. the degree of precision required for a particular purpose, such as a pharmaceutical formulation. For example, “about” can mean within 1 or more than 1 standard deviation, per the practice in the art. Alternatively, “about” can mean a range of up to 20%, preferably up to 10%, more preferably up to 5% and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

The term “drug” as used herein refers to a chemical entity of varying molecular size, small and large, naturally occurring or synthetic, that exhibits a therapeutic effect in animals and humans. A drug may include, but is not limited to, an organic molecule (e.g., a small organic molecule), a therapeutic protein, peptide, antigen, or other biomolecule, an oligonucleotide, an siRNA, a construct encoding CRISPR cas9 components and, optionally one or more guide RNAs, and the like.

A “pharmaceutically acceptable carrier” as used herein is defined as any of the standard pharmaceutically acceptable carriers. The pharmaceutical compositions of the subject invention can be formulated according to known methods for preparing pharmaceutically useful compositions. The pharmaceutically acceptable carrier can include diluents, adjuvants, and vehicles, as well as carriers, and inert, non-toxic solid or liquid fillers, diluents, or encapsulating material that does not react with the active ingredients of the invention. Examples include, but are not limited to: phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations are described in a number of sources that are well known and readily available to those skilled in the art. For example, Remington's Pharmaceutical Sciences (Martin E W [1995] Easton Pa., Mack Publishing Company, 19th ed.) describes formulations which can be used in connection with the drug delivery nanocarrier(s) (e.g., LB-coated nanoparticle(s)) described herein.

Phosphonolipids consist of 2-aminoethylphosphonic acid (ciliatine) residues attached to a lipid backbone, which can be either a ceramide, diacylglycerol or even a carbohydrate moiety of a glycolipid, i.e. the lipids have a carbon-phosphorus bond rather than carbon-oxygen-phosphorus bonds. Lipid-bound aminoethylphosphonic acid was first detected in the single-celled microorganism Tetrahymena pyriformis and then in protozoa. This proved to be a glycerophosphonolipid, but ceramide 2-aminoethylphosphonate, first found in sea anemones, and related sphingolipids are more often encountered and have been more studied. It is now recognized that organo-phosphonate compounds in general are widespread in nature, especially in microorganisms in the oceans, and that they make an appreciable contribution to the global biogeochemical cycle of phosphorus.

A “lipid compatible TLR7/8 agonist” as used herein refers to a TLR7/8 agonist that can be incorporated into a lipid bilayer without destabilizing that bilayer. In certain embodiments the lipid compatible TLR7/8 agonist is a hydrophobic molecule or a molecule comprising a hydrophobic domain or a tail. In certain embodiments the lipid compatible TLR7/8 agonist is a lipidated TLR7/8 agonist (e.g., in certain embodiments, the TLR7/8 agonist is coupled to a phospholipid or to a phosphonolipid).

### BRIEF DESCRIPTION OF THE DRAWINGS

FIG. 1 illustrates the structures of the lipidated TLR7/8 agonist 3M-052 (a.k.a. telratolimod) (top) and the lipoxin LXA4 (bottom).

FIG. 2, panels A-B, illustrates lipid bilayer encapsulation of the TLR7/8 agonist, 3M-052, in liposomes and silicasomes. Panel A depicts loading of lipophilic TLR7/8 agonist, 3M-052, into a lipid bilayer structure that could be applied to liposomes and silicasomes. Panel B depicts the synthesis of 3M-Liposome-IRIN (IRIN=Irinotecan).

FIG. 3, panels A-C, illustrates synthesis and preparation a co-formulated silicasome drug carrier to deliver a combination of 3M-052, plus the ICD-chemotherapeutic agents IRIN. Panel A) Schematic to outline the synthesis steps for constructing the silicasome. Briefly, MSNP was soaked in TEA₈SOS trapping agent at 65° C. (step 1), prior to adding to preheated (65° C.) pure ethanol, into which we dissolved a mixture of 3M-052 and lipids (DSPC/Chol/DSPE-PEG2000/3M-052, in the molar ratio of 55.5:38.5:2.7:3.3) (step 2). This mixture was sonicated to provide LB coating (step 3). For IRIN remote loading, irinotecan was dissolved in HEPES-buffered dextrose, before mixing and incubation of the purified TEA₈SOS-loaded 3M-silicasome at 65° C. (step 4). After quenching in ice water, the 3M-silicasome-IR was purified (step 5). As a control, we also synthesized a silicasome that incorporates 3M-052 only, without irinotecan loading (3M-silicasome-IR). Panels B-C) CryoEM visualization of the 3M-silicasome (panel B) and the 3M-silicasome-IR (panel C). The bar is 100 nm.

FIG. 4, panels A-D, shows examples of cryo-electron images of silicasomes and liposomes. Panel A depicts the 3M-Silicasome. Panel B shows the 3M-Silicasome-IRIN. Panel C shows the liposome structure and Panel D shows the 3M-Liposome-IRIN. Bar is 100 nm.

FIG. 5 shows a demonstration of a TLR7 agonist effect in HEK-Blue™ mTLR7 cells, which express both a copy of the murine TLR7 gene and an NF-κB/AP-1-inducible SEAP (secreted embryonic alkaline phosphatase) reporter gene.

FIG. 6, panels A-E, illustrates the therapeutic and synergistic drug effects of the dual delivery silicasome in a subcutaneous Kras pancreatic cancer (KPC) model. Panel A depicts the treatment outline for conducting the subcutaneous KPC tumor experiment. Panel B depicts the average subcutaneous KPC tumor growth kinetics, as determined by tumor volume. Panel C depicts the spaghetti growth curves (tumor volume) for each animal in each of the treatment groups. Panel D shows tumor reports in animal body weight over 20 days. Panel E shows photographic images of tumors in each treatment group on day 21. ***p<0.001.

FIG. 7, panels A-B, illustrates the presence and distribution of CD8+ cytotoxic T cells and FoxP3+ regulatory T cells (Treg) at the subcutaneous tumor sites. Panel A depicts the representative IHC images on day 21. Bar is 100 μm. Panel B depicts the quantitative data for CD8+ and FoxP3+ cells, as well as the ratio of the cell types. Data represents mean±SEM, n=5. *p<0.05; **p<0.01; ***p<0.001.

FIG. 8, panels A-G, illustrates the therapeutic and synergistic drug effects of the dual delivery silicasome in an orthotopic KPC pancreatic cancer model. Panel A depicts the experimental outline of the study conducted by orthotopic implantation KPC-luc tumor cells in the pancreatic tail of syngeneic mice. Panel B depicts the IVIS imaging intensity for each orthotopic tumor in each of the animal groups, recorded on days 7, 15, 18 and 21. Panel C depicts the average quantitative tumor bioluminescence in each group at day 21, as calculated by IVIS software. **p<0.01. Panel D depicts the IVIS imaging performed on the ex vivo explanted tumor tissues and potential metastatic organs on day 21. Panel E shows the average quantitative bioluminescence of the primary tumors in each group. Panel F depicts photographic images and primary tumor weights in each group shown, at the same level of magnification, on day 21. Panel G depicts animal body weights over a time period of 21 days.

FIG. 9, panels A-B, illustrates preparation of LXA4 embedded lipid bilayer for liposome synthesis. Panel A shows a schematic showing incorporation of lipophilic LXA4 into a lipid bilayer structure. Panel B shows a schematic showing synthesis methodology of liposomal LXA4 remotely loaded with irinotecan (IRIN).

FIG. 10, panels A-B, illustrates liposome characterization. Panel A) The liposomes were characterized for size and zeta potential and the drug loading capacity was determined with a ZETAPALS instrument (Brookhaven Instruments Corporation) and UV spectroscopy respectively. Panel B) Images of the resulting liposomes with cryo EM to determine the morphology, uniformity and structural integrity of the lipid bilayer coating. Scale bar is 100 nm.

FIG. 11, panels A-B, shows in vitro functional characterization of Lipo-LXA4-IRIN on human pancreatic stellate cells (hPSCs). Panel A) Schematic shows the TGF-β mediated cross-talk between cancer-associated fibroblasts (CAF) and cancer cells and involvement of cytokines such as IL6 that induces cancer cell growth (from Wu et al. (2021) Sig. Transduction & Targeted Therapy, 6: 218). Panel B) LXA4 mediated inhibition of IL6 secretion from TGF-β activated hPSCs detected from culture supernatant by ELISA.

FIG. 12, panels A-E, show in vivo therapeutic efficacy of the dual-delivery lipo-LXA4-IRIN in a subcutaneous KPC pancreatic cancer model. Panel A) Schematic outlines the implantation and treatment timeline of the subcutaneous KPC model. Panel B) Mean tumor volume across the treatment groups over the duration of the study depicting the growth kinetics. Panel C) Average body weights of the treated mice across the treatment groups depicting the overall health of the animal over the duration of the study. Panel D) Spaghetti plots showing the growth of individual tumors in each treatment group. Panel E) Images of the tumors that were excised upon termination of the experiment.

FIG. 13, panels A-B, shows the effect of LXA4 on the severity of desmoplasia estimated by collagen content in the KPC tumor microenvironment. Panel A) Images showing paraffin embedded tumor sections for each treatment group, sectioned at 4 μm stained for Masson's trichrome. Scale bar=500 μm. Panel B) Quantification of collagen positive areas, normalized over no treatment control. Data represents mean±SEM, n=5. Statistical analysis was performed by one way ANOVA. **p<0.01, ***p<0.001

FIG. 14, panels A-E, shows the effect of lipo-LXA4-IRIN on the immune potentiation estimated by immunohistochemistry for T lymphocytes in the KPC tumor microenvironment. Panel A) Representative IHC images of tumor sections stained for presence of CD8+ and FoxP3+ T cells. Scale bar=500 μm. Panels B and C) Quantification of the number of CD8+ and FoxP3+ T cells, expressed as a ratio of CD8+ to FoxP3+ T cells as assessed in the cores and margins of the tumors, respectively. Panel D) Representative IHC images of tumor sections stained for Perforin that serves as a marker for cytotoxic activity of CD8+ T cells. Scale bar=100 μm. Panel E) Quantification of perforin positive signals, normalized over no treatment control. Data represents mean±SEM, n=5. Statistical analysis was performed by one way ANOVA. **p<0.01, ***p<0.001,****p<0.0001.

FIG. 15 shows a schematic explaining the key design features for co-formulated drug delivery by lipid bilayer coated silicasomes and liposomes. The basic approach to drug coformulation is to use the hydrophilic interior of these carriers for remote loading of amphiphilic drugs, such as irinotecan, while employing the lipophilic bilayer to incorporate synthetic lipid moieties and prodrugs. The lipid moieties include synthetic agents with immune stimulatory activity, such as 3M-052 (a.k.a. Telratolimod). 3M-052 contains a C18 lipid tail that facilitates bilayer incorporation, first tested in a liposome to obtain optimal LB composition before applying that to the design of the MSNP bilayer. The schematic also shows that, in addition to 3M-052, the LB can be used, to incorporate a list of lipid-conjugated prodrugs that can provide immune checkpoint blockade or interfere in the immunometabolic IDO-1 pathway, as previously described by us.²² Irinotecan remote loading is accomplished by using ammonium sulfate or sucralose octasulfate for import into the aqueous interiors in the liposome or silicasome, respectively. These trapping agents allow amphipathic weak basic molecules (such as irinotecan, doxorubicin, and mitoxantrone) to cross the LB for protonation inside these carriers, where they collect as slowly dissolving drug precipitates. The hypothesis for the experimentation carried out in this communication is that the generation of immunogenic cell death by irinotecan will be boosted by co-delivery of 3M-052, leading to boosting of dendritic cell activation. Adapted with permission from ref 15. Copyright 2022 American Chemical Society

FIG. 16, panels A-F provides a demonstration of TLR7/8 agonist effect of the 3M-silicasome in vitro. Panel A) Schematic illustration of the TLR7-mediated signaling pathway, as portrayed for the HEK-Blue™ mTLR7 cell line. Panel B) Dose-dependent (fold) increase in activation of the SEAP transporter gene in HEK-Blue™ cells in response to treatment with a dose range of free R848, free 3M, and 3M-silicasome. Panel C) Flow cytometry analysis to quantify CD80 expression in the RAW264.7 macrophage cell line in response to the same stimuli, used at 10 μM. Panel D) ELISA results in the same cell line for IL-12p40 and TNF-α release in response to 10 μM of the same stimuli. Panel E) Flow cytometry analysis for CD11c expression on BMDCs by the same stimuli. Panel F) ELISA results for the same cytokines in cytokines in BMDCs as for (D). Data represent mean±SEM, n=3. ***p<0.001, ##p<0.01, ###p<0.001.

FIG. 17, panels A-D, illustrates the therapeutic impact on tumor growth in a subcutaneous KPC model. Panel A) Experimental timeline to assess the therapeutic impact of single and dual delivery carriers in subcutaneous KPC tumor-bearing mice. Panel B) Averaged tumor growth kinetics (n=6-7) through assessment of tumor volume in animals treated with saline, free 3M-052, free IRIN, 3M-silicasome, and 3M-silicasome-IR at dose equivalents of 2 mg/kg and 40 mg/kg for 3M-052 and IRIN, respectively, every 3-4 days, for a total of 4 IV injections. Please notice that the inhibition of tumor growth by free 3M-052, free IRIN, 3M-silicasome, and 3M-silicasome-IR is statistically significant compared to the saline control (p<0.001) on day 20. Although limited animal availability during COVID-19 precluded the inclusion of a carrier delivering encapsulated IRIN only, we have previously shown the advantage of an IRIN silicasome over liposomal or free drug delivery in KPC tumors.^{[7-9]} Panel C) photographic images of harvested tumors at the same magnification on day 21. Panel D). Spaghetti plots to depict the individual tumor growth kinetics over the course of the study. These plots further depict that inhibition of tumor growth by 3M-silicasome becomes statistically significant compared to free 3M-052 (p<0.05), from day 18 onwards. Moreover, inhibition of tumor growth by 3M-silicasome-IR becomes statistically significant from free IRIN and the 3M-silicasome (p<0.01) from day 8 onwards, and from free 3M-052 (p<0.01) from day 10 onwards. Data represent mean±SEM. #p<0.05, ##p<0.01, ###p<0.001.

FIG. 18, panels A-C, provides evidence of therapeutic anti-PDAC immunity in the subcutaneous pancreatic cancer model. KPC tumor-bearing mice were euthanized on day 21, followed by harvesting of the primary tumors and regional draining inguinal lymph nodes. Panel A) Representative IHC images and the quantification of CD8⁺ and FoxP3⁺ cell numbers at the primary tumor sites of different animal groups. Five primary tumors in each group were analyzed. Three fields were randomly selected for counting and the average counting number in each mouse was shown in the graphics. Bar is 100 μm. Data represent mean±SEM. Panel B) Representative flow cytometric analysis for quantitation of CD45+ dendritic cells in the lymph nodes, using the gating procedure described in online FIG. 27. Panel C) Flow cytometry analysis of the percentage CD80⁺CD86⁺ cells in CD11c⁺/CD45⁺ population. Data represent mean±SEM, *p<0.05; **p<0.01; ***p<0.001, ###p<0.001.

FIG. 19, panels A-B, illustrates pharmacokinetics and drug delivery by silicasomes versus free drugs. Healthy B6129SF1/J mice received one IV injection of free 3M-052, free IRIN, and 3M-silicasome-IR at dose equivalents of 2 mg/kg and 40 mg/kg for 3M-052 and IRIN, respectively. Blood collection was performed at 1, 5, 24, and 48 h post-injection (n=3). Free 3M-052 and IRIN were extracted by methanol, and plasma drug concentrations were measured by HPLC, as described in the experimental section. While free IRIN (panel A) and free 3M-052 (panel B) rapidly disappeared from the blood (i.e., <60 min, prompting the inserted graphs display), the 3M-silicasome-IR significantly increased both plasma IRIN and 3M-052 concentrations

FIG. 20, panels A-E, illustrates the use of DiR-labeled dual-drug silicasomes to assess in vivo biodistribution in an orthotopic KPC model. A DiR-labeled 3M-silicasome-IR nanocarrier was prepared for biodistribution assessment in an established KPC-derived orthotopic tumor model in B6129SF1/J mouse model. Tumor-bearing mice received one IV injection of saline, free IRIN, and DiR-3M-silicasome-IR at a dose equivalent of 40 mg/kg for IRIN. The in vivo and ex vivo IVIS imaging in the DiR label group were performed 24 and 48 h after IV injection, as well tumors were collected from all groups to determine IRIN contents by HPLC analysis. Panel A) Physicochemical properties of the DiR-labeled3M-silicasome-IR, including hydrodynamic size, PDI, and zeta potential. Panel B) Experimental timeline for the drug biodistribution study in orthotopic KPC tumor-bearing mice (n=3 or 4 animals). Panel C) In vivo and ex vivo fluorescence imaging and B6129SF1/J mice, 24 and 48 h after IV particle injection. The ex vivo IVIS image is representative of one of the animals in each of the 24 and 48 h animal groups. Additional ex vivo images appear online in FIG. 28. Panel D) Normalized fluorescence intensities, expressed as radiant efficiency, were calculated for all the explanted organs 24 and 48 h after IV injection. Panel E) The IRIN contents at the primary tumor sites were determined by HPLC analysis, as described in the experimental section. Data represent mean±SEM. ***p<0.001.

FIG. 21, panels A-G, shows a demonstration of synergistic drug effects in an orthotopic pancreatic cancer model. Panel A) Experimental timeline for tumor inhibition study of orthotopic KPC tumor-bearing mice (n=5). Panel B) IVIS images of individual tumors in each treatment group were obtained on days 7, 15, 18, and 21. Panel C) Use of IVIS software to calculate the average tumor bioluminescence intensity for each group on day 21. Panel D) Photographs and calculation of (panel E) average primary tumor weights in each group were obtained on day 21. Panel F) Ex vivo IVIS imaging of explanted organs from the orthotopic tumor-bearing mice were obtained on day 21. Panel G) Representative IHC images and the quantification of CD8+ and FoxP3⁺ cell numbers in the different treatment groups. 4 to 5 primary tumors in each group were analyzed. 3 fields were randomly selected for counting and the average counting number in each mouse was shown in the graphics. Bar is 100 μm. Data represent mean±SEM. **p<0.01; ***p<0.001, #p<0.05, ##p<0.01, p<0.001.

FIG. 22, panels A-C, shows incorporation of the TLR7/8 agonist, 3M-052, into a liposomal lipid bilayer. Panel A) Schematic of the lipid bilayer components that were used at different molar ratios to find an optimal composition for 3M-052 incorporation into the liposome. Panel B) Schematic to outline the synthesis steps for preparing a dual-drug liposome. Briefly, a mixture of 3M-052 and lipids (DSPC/Chol/DSPE-PEG2000/3M-052, in the molar ratio 55.5:38.5:2.7:3.3, was dissolved in CHCl3 (step 1) before solvent evaporation, with formation of a lipid film (step 2). The lipid film was hydrated in an ammonium sulfate buffer solution (pH 5.4) (step 3), followed by membrane extrusion (step 4) and finally buffer exchange by eluting across a desalting column (step 5). To accomplish IRIN remote loading, the drug was dissolved in HEPES-buffered dextrose, before mixing and incubation of the purified 3M-liposome at 65° C. (step 6). The reaction was quenched on ice water and the liposome purified (Step 7). The same procedure was used, to synthesize a liposome without Irinotecan loading. Panel C) Characterization of 3M-liposome and 3M-liposome-IR for hydrodynamic size, polydispersity index (PDI), zeta potential, and drug loading capacities.

FIG. 23, panels A-C, shows characterization of 3M-silicasome-DiD for hydrodynamic size and PDI (panel A), flow cytometry to demonstrate that BMDCs (panel B) and RAW 264.7 cells (panel C) participate in 3M-Si-DiD association/uptake at incremental doses over 21 h.

FIG. 24, panels A-D, illustrates cell viability testing, using an MTS assay, to demonstrate the absence of toxicity in RAW264.7 (panel A) and KPC (panel B) cell lines, exposed to 3M-Si at different concentrations for 48 h. Panel C) In vitro killing of KPC cells exposed to the different irinotecan concentrations for 48 h. Cytotoxicity was assessed by a CCK-8 assay. Panel D) Schematic to illustrate that the ICD response is mediated by two IRIN mechanisms of action, namely DNA damage due to its topoisomerase I inhibitory activity as well as induction of endoplasmic reticulum stress due to lysosomal alkalization. This ICD response is characterized by calreticulin (CRT) expression on the dying KPC cell surface, as well as HMGB1 and ATP release. CRT expression acts as an “eat-me” signal for DC which is also induced to mature by the adjuvant stimuli. Activated APCs migrate to the regional lymph nodes and the spleen to participate in the activation of cytotoxic T cells.^{[1]} Adapted with permission from ref 1. Copyright 2022 American Chemical Society.

FIG. 25 shows representative IHC images of CD8+ and FoxP3+ T-cell staining in subcutaneous tumors on day 21. The bar is 100 μm.

FIG. 26 shows a primary gating strategy for dendritic cell analysis. Singlet cells were selected from the cell population, with the exclusion of dead cells. Following gating of CD11c+CD45+ dendritic cells, the selected subset was subsequently analyzed for the expression of the costimulatory surface receptors, CD80 and CD86. FSC-H, forward scatter height; FSC-A, forward scatter area.

FIG. 27 shows body weight measurements of mice during treatment with saline, free 3M-052, free IRIN, 3M-silicasome, and 3M-silicasome-IR for 20 days.

FIG. 28 shows IVIS imaging of explanted organs 24 and 48 hours after a single IV injection of DiR-labeling 3M-silicasome-IR, delivering dose equivalents of 2 mg/kg and 40 mg/kg, respectively, for 3M-052 and IRIN (n=4).

FIG. 29, panels A-B, show IVIS imaging of explanted organs from individual orthotopic tumor-bearing mice in each treatment group, following sacrifice on day 21 (n=4-5) (panel A) and physicochemical characterization of the silicasome-IR for hydrodynamic size and PDI (panel B).

FIG. 30 shows representative IHC images of CD8+ and FoxP3+ T-cell staining in orthotopic tumors, harvested on day 21. The bar is 100 μm.

FIG. 31 shows body weight assessment of mice after treatment with saline, free IRIN, 3M-silicasome, silicasome-IR, and 3M-silicasome-IR.

FIG. 32 shows illustrative TLR agonists that can be included in the LB of nanocarriers (e.g., liposomes and silicasomes).

### DETAILED DESCRIPTION

In various embodiments provided herein are liposomes and silicasomes (lipid bilayer coated porous silica nanoparticles) that co-deliver a chemotherapeutic agent (such as Irinotecan) with a lipid compatible TLR7 and/or TLR8 (e.g., TLR7/8) agonist (such as 3M-052) and/or with a lipoxin (e.g., LXA4) to mount a synergistic anti-tumor (e.g., anti-PDAC) immune response. In certain embodiments the chemotherapeutic agent is an agent that induces immunogenic cell death (ICD inducer).

Accordingly, we envisaged Irinotecan (and/or other agents) inducing an immunogenic cell death response (that includes the release of a TLR4 agonist (HMGB1) at the newly generated “hot” TME, which can be further propagated by the impact of a co-delivered TLR7/TLR8 agonist (e.g., 3M-052). More generally, in light of the immunogenic properties of Irinotecan, we postulated that a silicasome (or liposome) that co-delivers both a chemotherapeutic agent (e.g., ICD inducer), as well as a TLR7/8 agonist could act synergistically to improve the anti-PDAC immune response. Thus, we posited that Irinotecan (or other ICD inducer) will induce immunogenic cell death (which includes the delivery of an endogenous TLR4 signal in the form of HMGB-1), which with the action of a co-delivered TLR7/8 agonist (e.g., 3M-052) would further strengthen the immune response in the newly generated “hot” TME by stimulating TLR7 and/or TLR8. It is possible to envisage multiple mechanisms of synergy, including integration of the immune activation pathways of surface membrane associated TLR4 with endosomal TLR7 and 8 in multiple cell types. It is also possible that the synergy can take place at the level of the regional lymph nodes, where TLR7 also exert a robust adjuvant effect on T-cell activation and as a component of the cancer immunity cycle.

It is also noted use of lipid compatible TLR7/8 agonists incorporated into the lipid bilayers of the drug delivery vehicles described herein can not only limit systemic toxicity, but the encapsulation into a nanocarrier could also improve the pharmacokinetics of TLR7/8 agonist (e.g., 3M-052) delivery to the tumor site to improve innate and cognate immune activity, including activation and recruitment of cytotoxic T cells, while also interfering in immune suppressive cells. In this regard, 3M-052, and other TLR7/8 agonists, have also been shown to reprogram tumor associate macrophages from an M2-dominant to M1-dominant phenotype. A further advantage of the drug delivery vehicles (nanocarriers) described herein is to utilize the lipid bilayer for remote loading of ICD inducing chemotherapeutics (e.g., Irinotecan), leading to dual drug delivery with harmonized PK and the potential for the TLR7/8 agonist to synergize with the immunogenic death response in the same local regional TME domain.

It is also recognized that pancreatic ductal adenocarcinoma (PDAC) consists of transformed cells, immune cells as well as a non-transformed stroma that accounts for 70-90% of tumor mass. The tumor microenvironment is a crucial factor in the pathobiology and progression of PDAC. Pancreatic stellate cells (PSCs) are myofibroblast-like cells in the pancreas that interact with transformed cells and mount a dysregulated wound healing response. The resulting fibrosis progresses to generate the desmoplastic stroma which in turn modulates immune evasion, proliferation, EMT, migration and invasion of pancreatic cancer cells. PSCs are activated by a plethora of molecules including transforming growth factor-β (TGF-β) which is released by cancer cells and immune cells. Upon activation, PSCs secrete cytokines such as IL6, IL1β and TGF-β as paracrine signals to cancer cells to sustain proliferation, migration, and invasiveness. PSCs are not passive bystanders but pro-inflammatory, tumor-supporting and therefore, warrant therapeutic targeting alongside tumor cells to establish treatment longevity and improve patient outcome.

Lipoxins belong to the first recognized class of anti-inflammatory lipids that function as endogenous “stop signals”, impeding the deleterious responses of PMNs and regulating excessive leukocyte trafficking. Lipoxins are transiently and locally secreted by immune cells such as neutrophils and macrophages in response to injury or inflammation.

Lipoxins and epi-lipoxins bind to the high-affinity G protein-coupled lipoxin A4 (LXA4) receptor formyl peptide receptor 2 (FPR2)/ALX to resolve inflammation at nanomolar concentrations. There are several lines of evidence indicating possible anti-fibrotic properties of LXA4. It has been studied to inhibit connective tissue growth factor-induced proliferation and to interfere with TGF-β dependent pro-fibrotic properties of lung myofibroblasts. LXA4 also attenuated experimental renal fibrosis and inhibited epithelial to mesenchymal transition of renal epithelial cells in proximal tubules. Presently, it's role in reducing desmoplasia is being explored for anti-cancer application to inhibit cancer progression and metastasis in pancreatic tumors.

LXA4 is rapidly metabolized by human monocytes by dehydrogenation and reduction to 13,14-dihydro LXA4. Therefore, the quest for stable and hydrophilic analogs of LXA4 to resist rapid enzymatic inactivation and to prolong their duration of action is of great relevance. Commercially, lipoxins are available as a solution in EtOH, DMF or DMSO. We incorporated LXA4 into a liposomal bilayer (e.g., in a liposome or silicasome) to develop a stable, injectable, sustained-release formulation with room for remote-loading of a chemotherapeutic for targeted therapy in PDAC. Considering the immunogenic properties of irinotecan (or other ICD inducers), we proposed that the development of a dual-delivery liposome (or silicasome) comprising a chemotherapeutic agent, and a pro-resolving, anti-fibrotic lipid could present a therapeutic strategy for, inter alia, the following reasons:
(i) LXA4 mediated attenuation of the TGF-β pathway in the TME will desist activation of tumor resident PSCs, potentially reducing stromogenesis.
(ii) By alleviating the stromal burden, the LXA4-irinotecan liposomes would enhance sensitivity of tumor cells to the ablative effects of irinotecan as well as enhance intratumoral trafficking of immune cells.
(iii) Induction of ICD (e.g., irinotecan induced ICD) would potentiate training of antigen-specific cytotoxic T cells, thus enabling regression of primary and metastatic lesions.

It will be recognized that, in certain embodiments the drug delivery carrier can comprise both a lipoxin and a TLR7/8 agonist along with a chemotherapeutic drug (e.g., an ICD inducer such as irinotecan (IRIIN)).

Accordingly, in various embodiments, a drug delivery vehicle for the co-delivery of a chemotherapeutic agent and a TLR7/8 agonist and/or a lipoxin is provided where the vehicle comprises:
a) a silicasome comprising a porous nanoparticle encapsulated in a lipid bilayer, where the lipid bilayer contains a lipoxin and/or a lipid compatible TLR7/8 agonist disposed in the bilayer and a chemotherapeutic agent (e.g., an ICD inducer) contained in pores comprising the porous nanoparticle and said chemotherapeutic agent comprises a chemotherapeutic agent that induces immunogenic cell death (ICD); or
b) liposome comprising a lipid bilayer where the lipid bilayer contains a lipoxin and/or a TLR7/8 agonist disposed in the bilayer, and a chemotherapeutic agent (e.g., an ICD inducer) inside the liposome.

Also provided are pharmaceutical formulations comprising the drug delivery carrier, kits containing the drug delivery carrier as well as methods of use of the drug deliver carrier for the treatment of a cancer.

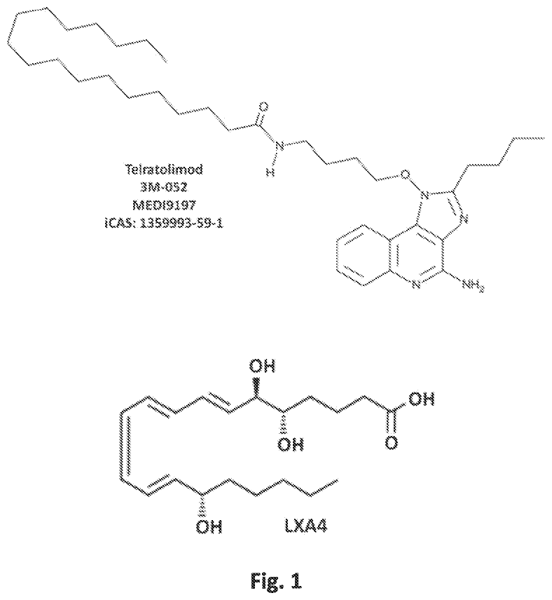

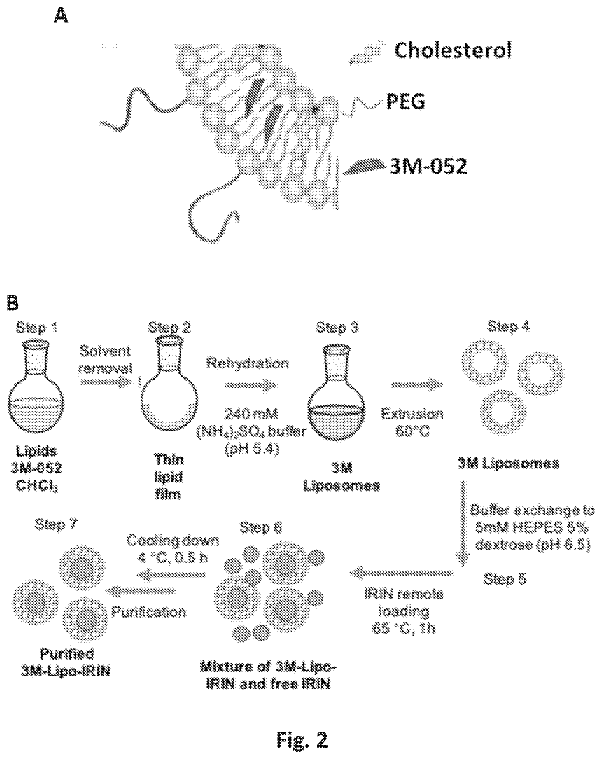

We note that proof of principle is provided in the Examples that show drug delivery vehicles that co-deliver the lipidated TLR7/8 agonist telratolimod (see, e.g., FIG. 1) and the chemotherapeutic agent irinotecan (IRIN) (Example 1) and that show drug delivery vehicles that co-deliver the lipoxin (LXA4) (see, e.g., FIG. 1) and the chemotherapeutic agent irinotecan (IRIN) (Example 2).

However using the teachings provided herein drug delivery vehicles that deliver both a TLR7/8 agonist and a lipoxin in combination with a chemotherapeutic drug and/or that comprise TLR7/8 agonists, lipoxins, and chemotherapeutic agents other than Irinotecan.

### Toll-Like Receptor (TLR) Agonists for Use in the Silicasomes and Liposomes Described Herein.

One strategy to turn an immunologically cold tumor hot is to promote activation of antigen presenting cells (APC) by targeting the endosomal Toll Like Receptors (e.g., TLR3, TLR7, TLR8, TLR9, etc.). TLR3, TLR7, TLR8, and TLR9 recognize single and double stranded viral RNA and bacterial CpG DNA in the endosome following internalization by APCs. TLR signaling activates APCs, increasing expression of inflammatory cytokines and co-stimulatory molecules, and enhancing antigen presentation capacity. Thus, APC activation by TLRs can promote switching of CD4+ T cell response from Th2 to Th1, enhance CD8+ T cell responses, and inhibit T regulatory cell responses (see, e.g., Pasare et al. (2004) Microbes Infect. 6(15): 1382-1387; Peng et al. (2005) Science, 309(5739): 1380-1384; Tomai et al. (2000) Cell Immunol. 203(1): 55-65; Vasilakos et al. (2000) Cell Immunol. 204(1): 64-74).

Thus, as indicated above, in various embodiments the drug delivery vehicles described herein comprises TLR7 and/or TLR8 agonists (TLR7/8 agonists). In certain embodiments the TRL7/8 agonist comprises a lipid compatible TLR7/8 agonist. In certain embodiments the TRL7/8 agonist comprises a lipidated TRL7/8 agonist.

A TLR7/8 agonist has the potential to activate a broad range of human APCs within the tumor microenvironment. TLR7 is expressed on plasmacytoid dendritic cells (pDCs) and B cells, while TLR8 is more widely expressed on monocytes and myeloid dendritic cells (mDCs) (Kadowaki et al. (2001) J. Exp. Med. 194(6): 863-869).

It was hypothesized that retention of the TLR7/8 agonist in the tumor may be important for efficacy. Moreover, without being bound to a particular theory, it is believed that a TL7/8 agonist when co-delivered with an inducer if immunogenic cell death (ICD) inducer can act synergistically with the ICD inducer to facilitate a strong anti-tumor immune response in the tumor microenvironment. In certain embodiments the TLR7/8 agonist facilitates transition of the tumor microenvironment (TME) from immunologically cold to hot to thereby facilitate/synergize the activity of the ICD inducer.

Accordingly as indicated above, in various embodiments, a drug delivery carrier that comprises both a TLR7/8 agonist and a chemotherapeutic drug, in certain embodiments a chemotherapeutic drug that is an ICD inducer is provided.

Lipid compatible TLR7/8 agonists are well known to those of skill in the art and include but are not limited to lipidated versions of known TLR7/8 agonists. The experiments and results described herein utilize the lipidated TLR7/8 agonist 3M-052 (see, e.g., FIG. 1, top)). However, it is believed that other lipidated TLR7/8 agonists can function in a similar manner in the drug delivery vehicles described herein.

Such lipidated TLR7 agonists include, but are not limited to lipidated imidazoquinoline derivatives (IMDs) such as a lipidated imiquimod, a lipidated resiquimod, a lipidated 852-A PF-4878691), and the like, a lipidated pteridinone-based TLR7/8 agonist such as lipidated vesatolimod (GS-9620), lipidated 8-oxoadenine (AZD-8848), and the like, and lipidated TLR8-specific benzazepine such as lipidated motolimod (VTX-2337), a lipidated and pyrimidine such as lipidated selgantolimod (GS-9688), and the like.

Illustrative, but non-limiting, examples of lipidated TLR7/TLR8 agonists are described by Miller et al. (2020) Front. Immunol., Vol. 11, Art. 406, doi: 10.3389/fimmu.2020.00406. Illustrative TLR7/8 agonists described therein that can be used in the presently described drug delivery vehicles (drug carriers) include lipidated versions of UM-3001 shown below.

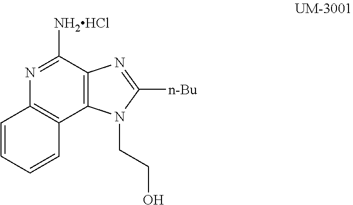

One example of such is shown by the formula:

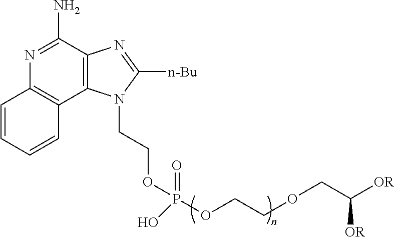

where n is 3-15 and R is C(O)(CH₂)₁₄CH₃. In certain embodiments n is 3, 6, 9, or 12 and R is C(O)(CH₂)₁₄CH₃. In certain embodiments the compound is UM-3003 (Miller et al. supra) in which n is 3 and R is C(O)(CH₂)₁₄CH₃, or UM-3004 in which n is 6 and R is C(O)(CH₂)₁₄CH₃.

In certain embodiments the TLR7/TLR8 agonist is UM-3005 having the formula shown below.

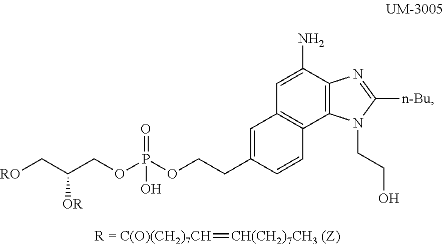

in which R is C(O)(CH₂)₇CH═CH(CH₂)₇CH₃.

Other suitable TLR7/8 agonists are described, inter alia, in U.S. Pat. Nos. 8,946,421 and/or 8,624,029 which are incorporated herein by reference in their entirety for all purposes. Typically, these compounds comprise an imidazoquinoline molecule that may be covalently linked to a phospho- or phosphonolipid group. In certain embodiments the agonists are broadly described by Formula I:

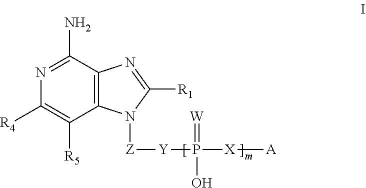

where R₁ is H, C₁₋₆ alkyl, C₁₋₆ alkylamino, C₁₋₆ alkoxy, C₃₋₆ cycloalkylC₁₋₆ alkyl, C₃₋₆cycloalkylC₁₋₆alkylamino, C₃₋₆cycloalkylC₁₋₆ alkoxy, C₁₋₆alkoxyC₁₋₆alkyl, C₁₋₆ alkoxyC₁₋₆alkylamino, C₁₋₆alkoxyC₁₋₆ alkoxy; branched or unbranched and optionally terminally substituted with a hydroxyl, amino, thio, hydrazino, hydrazido, azido, acetylenyl, carboxyl, or maleimido group, Z is C₂-C₆ alkyl or alkenyl, unsubstituted or terminally substituted by —(O—C₂-C₆ alkyl)₁₋₆, Y is Y is O, or NH, X is X is O, CH₂, or CF₂, W is O or S, m is 1 or 2, and A is

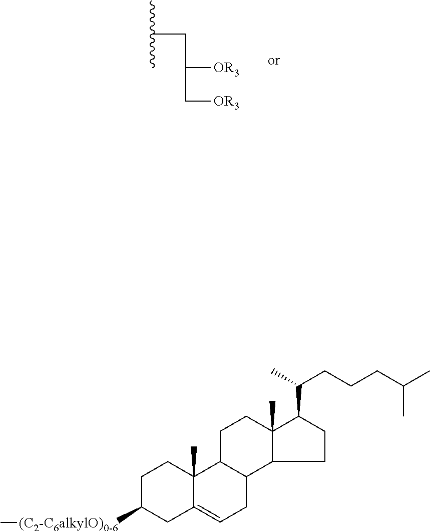

where R₂ is straight/branched/unsaturated C₄-C₂₄ alkyl or acyl, and R₃ is straight/branched/unsaturated C₄-C₂₄ alkyl or acyl, R₄ and R₅ are independently H, C₁-C₆ alkyl, C₁-C₆ alkoxy, halogen, or trifluoromethyl; or taken together alternatively form a 6-membered aryl, heteroaryl containing one nitrogen atom, cycloalkyl, or heterocycloalkyl ring containing one nitrogen atom unsubstituted or substituted by one or more of C1-C₆ alkyl, C₁-C₆ alkoxy, halogen, or trifluoromethyl, or pharmaceutically acceptable salts thereof.

In one embodiment, these lipidated TLR7/8 agonists are more specifically described by Formula II:

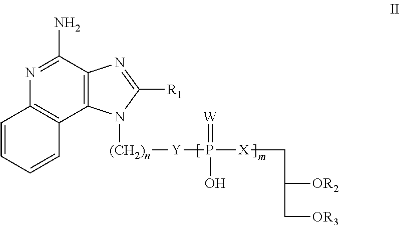

in which R₁ is H, C₁_₆ alkyl, C₁_₆ alkylamino, C₁_₆ alkoxy, C₃₋₆cycloalkylC₁₋₆ alkyl, C₃₋₆cycloalkylC₁₋₆alkylamino, C₃₋₆cycloalkylC₁₋₆ alkoxy, C₁₋₆ alkoxyC₁₋₆ alkyl, C₁₋₆ alkoxyC₁₋₆ alkylamino, C₁_₆ alkoxyC₁₋₆alkyl; branched or unbranched and optionally terminally substituted with a hydroxyl, amino, thio, hydrazino, hydrazido, azido, acetylenyl, carboxyl, or maleimido group, n is 1-6, Y is O or NH, X is O, CH₂, or CF₂, W is O or S, m is 1-2, R₂ is H or straight/branched/unsaturated C₄-C₂ alkyl or acyl, R₃ is straight/branched/unsaturated C₄-C₂₄ alkyl or acyl (e.g. phosphatidyl, lysophosphatidyl ether or ester, and when W is O, X is O and M is 1.

In certain embodiments the compound is a compound of Formula II where R1 is H, n-butyl, or ethoxymethyl, n is 2-4, X is O, Y is O, W is O, m is 1-2, and both R₂ and R₃ are hexadecanoyl.

In one illustrative embodiment, the lipidated TLR7/TLR8 agonist comprise compound L4 from U.S. Pat. No. 8,624,029 B2 (4-amino-1-[2-(1,2-dipalmitoyl-sn-glycero-3-phospho)ethyl]-1H-imidazo[4,5-c]quinoline (Formula (I) where R1 is H, Y, W, and X are O, n is 2, m is 1, R₂ and R3 are n-C₁₅H₃₁CO):

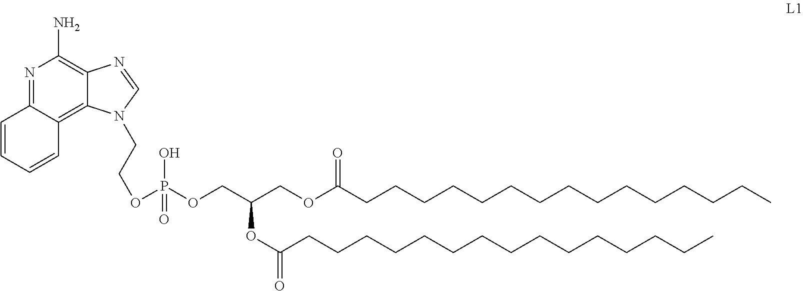 [SMILES: CCCCCCCCCCCCCCCC(=O)OC[C@H](COP(=O)(O)OCCn1cnc2c(N)nc3ccccc3c21)OC(=O)CCCCCCCCCCCCCCC]

In one illustrative embodiment, the lipidated TLR7/TLR8 agonist comprise compound L4 from U.S. Pat. No. 8,624,029 B2 (4-amino-1-[2-(1,2-dipalmitoyl-sn-glycero-3-phospho)ethyl]-2-butyl-1H-imidazo[4,5-c]quinoline (Formula I in which R₁ is n-C₄H₉, Y, W, and X are all O, n is 2, m is 1, and R₂ and ═R₃ are both n-C₁₅H₃₁CO):

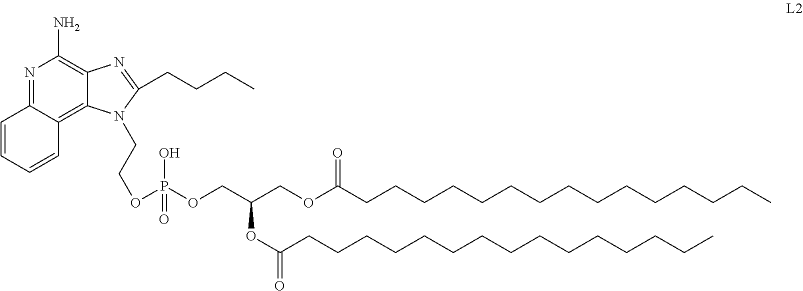 [SMILES: CCCCCCCCCCCCCCCC(=O)OC[C@H](COP(=O)(O)OCCn1c(CCCC)nc2c(N)nc3ccccc3c21)OC(=O)CCCCCCCCCCCCCCC]

In one illustrative embodiment, the lipidated TLR7/TLR8 agonist comprise compound L4 from U.S. Pat. No. 8,624,029 B2 (4-Amino-1-[2-(1,2-dipalmitoyl-sn-glycero-3-phospho)ethyl]-2-ethoxymethyl-1H-imidazo [4,5-c]quinoline (Formula I where R₁ is CH₂OCH₂CH₃, Y, W, and X are all O, n is 2, m is 1, and R₂ and R₃ are both n-C₁₅H₃₁CO):

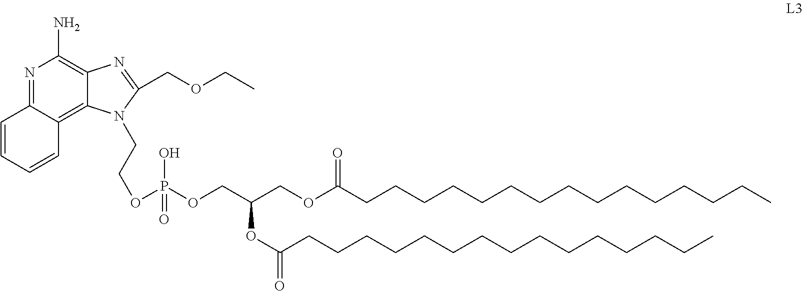 [SMILES: CCCCCCCCCCCCCCCC(=O)OC[C@H](COP(=O)(O)OCCn1c(COCC)nc2c(N)nc3ccccc3c21)OC(=O)CCCCCCCCCCCCCCC]

In one illustrative embodiment, the lipidated TLR7/TLR8 agonist comprise compound L4 from U.S. Pat. No. 8,624,029 B2 (4-Amino-1-[2-(1,2-dipalmitoyl-sn-glycero-3-phospho)butyl]-2-ethoxymethyl-1H-imidazo[4,5-c]quinoline (Formula 1 where R₁ is H, Y, W, and X are all O, n is 4, m is 1, and R₂ and R₃ are both n-C₁₅H₃₁CO):

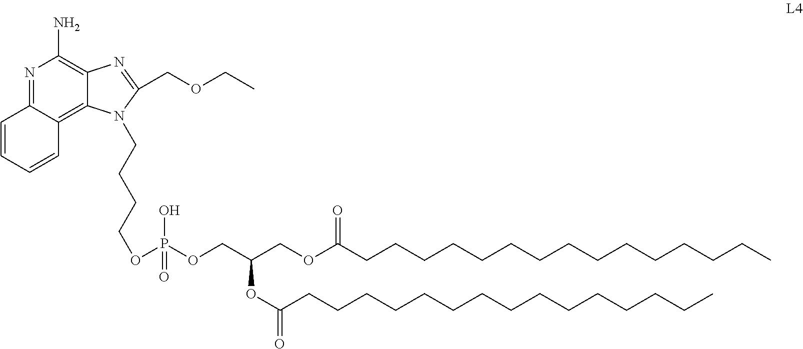 [SMILES: CCCCCCCCCCCCCCCC(=O)OC[C@H](COP(=O)(O)OCCCCn1c(COCC)nc2c(N)nc3ccccc3c21)OC(=O)CCCCCCCCCCCCCCC]

In one illustrative embodiment, the lipidated TLR7/TLR8 agonist comprise compound L5 from U.S. Pat. No. 8,624,029 B2 4-amino-1-[2-(1,2-dipalmitoyl-sn-glycero-3-diphospho)ethyl]-2-ethoxymethyl-1-1H-imidazo[4,5-c]quinoline (Formula I wherein R₁ is CH₂OCH₂CH₃, Y, W, and X are all O, n is 2, m is 2, and R₂ and R₃ are both n-C₁₅H₃₁CO):

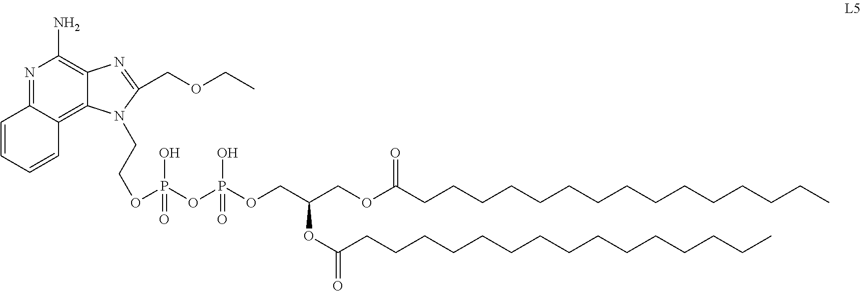 [SMILES: CCCCCCCCCCCCCCCC(=O)OC[C@H](COP(=O)(O)OP(=O)(O)OCCn1c(COCC)nc2c(N)nc3ccccc3c21)OC(=O)CCCCCCCCCCCCCCC]

With respect to the TLR7/8 agonists, it is also noted that TLR7 agonists also exert adjuvant effects. Nanocarriers (drug delivery vehicles) that include these molecules can show activity at the level of lymph nodes, in addition to the action at the primary cancer site. For instance, Bhagchandani et al. (2021) Adv. Drug Deliv. Rev., 175: 113803. Indicates that particulate TLR7/8 conjugates showed enhanced lymph node cytokine production and uptake by migratory APCs as well as an order of magnitude increase in the influx of CD11c+ DCs and monocytes in the draining LN compared with soluble forms. The draining lymph node actively contributes to the immunogenic cell death effect leading to T-cell activation (by providing antigen ingested dendritic cells), before returning those T cells to the primary tumor site. Thus, it is believed the TLR7 agonists, and accordingly the drug delivery vehicles described herein comprising TLR7/8 agonists, can exert adjuvant effects at two separate but complementary localities in the so-called cancer immunity cycle, namely at the primary tumor site, as well as the participating draining lymph node.

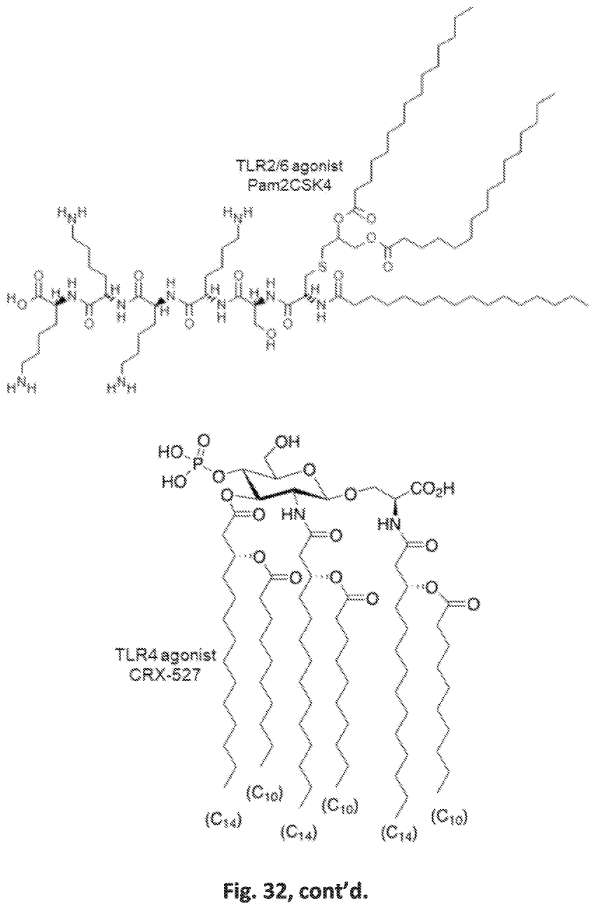

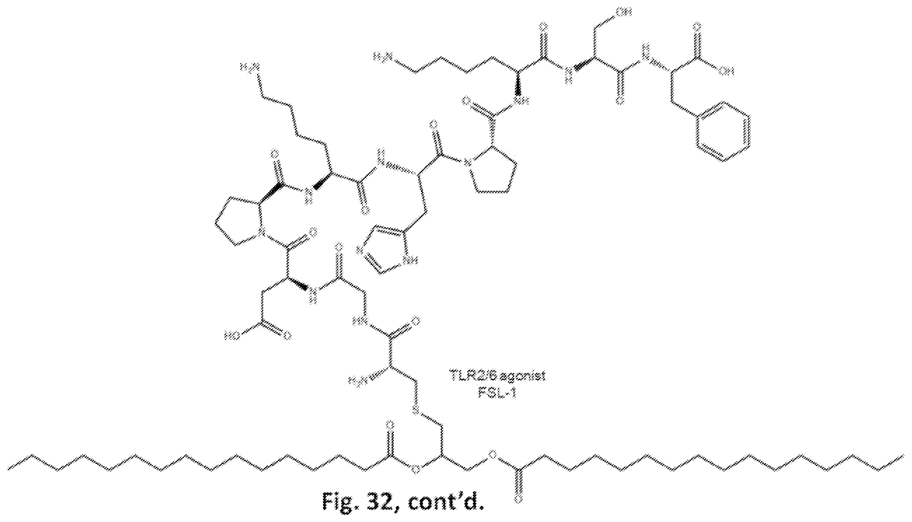

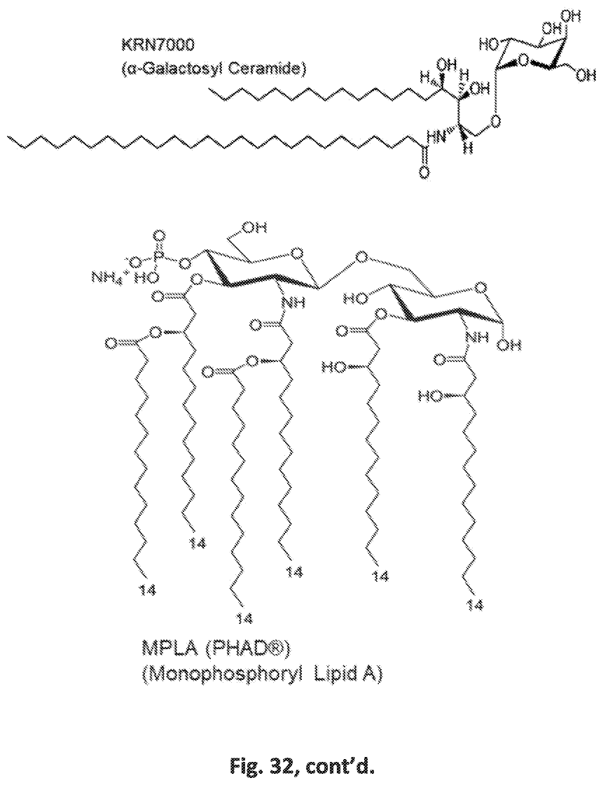

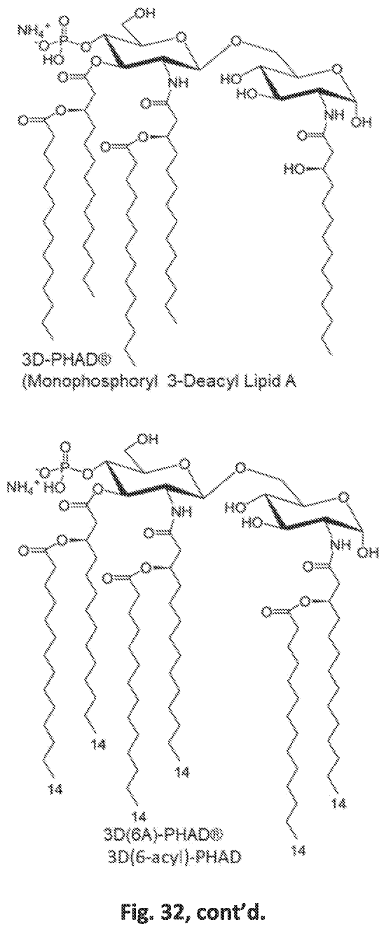

Other suitable TLR agonists that can be included in a lipid bilayer are shown in Table 1 and FIG. 32.

TABLE 1
Illustrative TLR agonists that can be included in the
LB of nanocarriers (e.g., liposomes and silicasomes).

| Category | Drug | Activity | Reference |
|---|---|---|---|
| TLR4 | MPLA | Monophosphoryl Lipid A | Wang et al. (2020) Eur. |
| agonists | (PHAD ®) | (Synthetic), TLR4 agonist, | J. Pharm. Biopharm. |
| | | induces proinflammatory | 147: 76-86. |
| | | cytokines and antigen-specific | Wu et al. (2021) |
| | | effector CD4⁺ and memory | Bioeng. Transl. Med. |
| | | CD8⁺ T cells. Administration | 6(1): e10188 |
| | | to human subjects without | Chatzikleanthous, et al. |
| | | serious adverse effects. | (2021) Mol. |
| | | Excellent efficacy of | Pharmaceutics, 18(8): |
| | | doxorubicin-MPLA | 2867-2888 |
| | | liposomes against 4T1 | |
| | | tumors. Additional analogs | |
| | | include: | |
| | | 3D-PHAD ® | |
| | | (Monophosphoryl 3-Deacyl | |
| | | Lipid A): less pyrogenic | |
| | | 3D(6A)-PHAD ® [3D(6- | |
| | | acyl)-PHAD ®]: Adjuvant | |
| | | used in GSK's Adjuvant | |
| | | Systems AS01, AS02, and | |
| | | AS04, including for use in | |
| | | liposomes | |
| TLR7/8 | MEDI9197; | Injectable, tissue-retained | Singh, J Immunol. |
| | 3M-052 | agonist that forms a tissue | 2014 Nov. 1; 193(9): |
| | (Telratolimod) | depot with gradual, sustained | 4722-4731. |
| | | release, allowing local TLR | Bhagchandani, ADDR |
| | | activation without systemic | 2021 |
| | | cytokine release; induces | |
| | | local innate immune | |
| | | activation as well as systemic, | |
| | | antigen-specific CD8+ T cell | |
| | | responses which suppress | |
| | | tumor growth; currently under | |
| | | clinical development for use | |
| | | in vaccines and cancer | |
| | | therapy. | |
| | | Design strategies for | |
| | | nanoparticles construction | |
| | | include self-assembly | |
| | | conjugate formulations, | |
| | | conjugation of protein | |
| | | antigens to copolymers or | |
| | | P(Man-TLR7) covalently | |
| | | conjugated onto synthetic | |
| | | polymer scaffolds | |
| α-Galactosyl- | KRN7000 | Synthetic analog and ligand of | //avantilipids.com/product/867000 |
| ceramide | | the lipid-binding MHC class | |
| analogues | | I-like protein, CD1d. Displays | |
| | | potent antitumor activity in | |
| | | various in vivo models. | |
| TLR4 agonist | Kdo2-Lipid A | Kdo2-Lipid A ammonium is a | Saito et al. (2010) |
| | ammonium | chemically defined | Br J Pharmacol. |
| | | lipopolysaccharide (LPS) with | 160(7): 1754-1764. |
| | | endotoxin activity equal to | |
| | | LPS. Kdo2-Lipid A | |
| | | ammonium is highly selective | |
| | | for TLR4. Kdo2-Lipid A | |
| | | ammonium stimulates the | |
| | | release of both TNF and | |
| | | PGE2. | |
| | | Stimulates the release of both | |
| | | TNF and PGE2 from the adult | |
| | | rat spinal astrocytes cell | |
| | | cultures. | |
| | | Activates toll-like receptor 4 | |
| | | (TLR4) in vivo. | |
| TLR2/TLR6 | Pam2CSK4 | Synthetic diacylated | Parra-Izquierdo et al. |
| agonist | | lipopeptide (LP) and a potent | (2021) Front Immunol. |
| | | activator of the pro- | 2: 729951. |
| | | inflammatory transcription | Kang et al. (2009) |
| | | factor NF-κB. Pam2CSK4 | Immunity, 31(6): 873-84. |
| | | mimics the acylated amino | Noh et al. (2015) |
| | | terminus of bacterial LPs and | Mol. Immunol. 64(1): |
| | | is recognized by the | 183-189. |
| | | TLR2/TLR6 heterodimer. | |
| TLR2/TLR1 | Pam3CSK4 | Synthetic triacylated | Aliprantis et al. (1999) |
| agonist | | lipopeptide (LP) and a | Science, 285(5428): |
| | | TLR2/TLR1 ligand. | 736-739. |
| | | A potent activator of the pro- | Oliveira-Nascimento et |
| | | inflammatory transcription | al. (2012) Front Immunol. |
| | | factor NF-κB. | 3: 79. |
| | | Mimics the acylated amino | Ozinsky et al. (2000) |
| | | terminus of bacterial LPs. | Proc. Natl. Acad. Sci. |
| | | | USA,, 97(25): 13766- |
| | | | 13771. |
| TLR2/TLR6 | FSL-1 | Synthetic lipoprotein derived | Shibata et al. (2000) |
| agonist | | from Mycoplasma salivarium. | J. Immunol. 165: 6538- |
| | | Contains a diacylated cysteine | 65-44. |
| | | residue and is recognized by | Okusawa et al. (2004) |
| | | the TLR2/TLR6 heterodimer. | Infect. Immun. 72(3): |
| | | | 1657-1665. |
| | | | Takeuchi et al. (2001) |
| | | | Int. Immunol. 13(7): |
| | | | 933-40. |
| TLR4 | CRX-527 | Synthetic lipid A mimic | Stover et al. (2003) |
| agonist | | belonging to the aminoakyl | J. Biol. Chem. 279: |
| | | glucoaminide 4-phosphate | 4440-4449. |
| | | (AGP) family. | Legat et al. (2010) |
| | | Initiates MyD88- and TRIF- | Eur. J. Immunol. 40: |
| | | dependent pro-inflammatory | 792-802. |
| | | signaling cascades. | Tan et al. (2013) |
| | | | Open J. Immunol. 3: 1-9. |
| Pro-resolving | LXA4 | Autacoid metabolite of | Zong et al. (2017) |
| lipid | | arachidonic acid, interacting | J. Exp. Clin. Cancer Res. |
| mediators | | with lipoxygenase and acting | 36: Art. Num: 181 |
| | | primarily to dampen | |
| | | inflammation. Downregulates | |
| | | TGF-β1 autocrine signaling, | |
| | | with reversal of mesenchymal | |
| | | phenotypes and metastases in | |
| | | PDAC. | |
| | Resolvins (D | Lipid autacoids with anti- | Hallisey et al. (2020) |
| | series 1-6) | angiogenic properties that | FASEB J., 34(S1) |
| | | reduce local inflammatory | |
| | | cytokines by macrophage | |
| | | mediated clearing of cellular | |
| | | debris, thereby inhibiting | |
| | | tumor cell proliferation. | |
| | Resolvins (E | Apoptotic pro-tumorigenic | Sulciner et al. (2018) |
| | series 1-6) | cellular debris are cleared out | J. Exp. Med. 215(1) |
| | | in situ by resolving-mediated | |
| | | recruitment of TIM4+ and | |
| | | CD11b+ phagocytic | |
| | | macrophages. | |

The foregoing lipid compatible TLR agonists are illustrative and non-limiting. Using the teaching provided herein, numerous drug delivery vehicles comprising other TLR7/8 agonists disposed in a lipid bilayer will be available to one of skill in the art.

### ICD Inducers for Use in the Silicasomes and Liposomes Described Herein.

In various embodiments the liposomes or silicasomes described herein contain (e.g., within the aqueous core of the liposome of within the pores of the nanoparticle comprising the silicasome) one or more inducers of immunogenic cell death (ICD inducers). ICD inducers are well known to those of skill in the art and include but are not limited to mitoxantrone (MTX), doxorubicin (DOX), oxaliplatin, anthracenedione, bleomycin, bortezomib, cisplatin, daunorubicin, docetaxel, epirubicin, idarubicin, paclitaxel, R2016, cyclophosphamide, irinotecan, and the like (see, e.g., PCT Publication Nos: PCT/US2018/033265 (WO 2018/033265), PCT/US2020/055585 (WO 2021/076630), and the like).

Additional ICD inducers include but are not limited to nanomaterials that induce ICD. Such nanomaterials include, but are not limited to CuO, Cu₂O, Sb₂O₃, As₂O₃, Bi₂O₃, P₂O₃, ZnO, TiO₂, graphene oxide, and 2D materials other than graphene or graphene oxide.

The foregoing ICD inducers are illustrative and non-limiting. Using the teaching provided herein, numerous drug delivery vehicles comprising other ICD inducers will be available to one of skill in the art.

### Lipoxins for Use in the Silicasomes and Liposomes Described Herein.

In various embodiments the drug delivery vehicles described herein (e.g., silicasomes and/or liposomes) comprise a lipoxin disposed in the lipid bilayer. Lipoxins belong to the first recognized class of anti-inflammatory lipids that function as endogenous “stop signals”, impeding the deleterious responses of PMNs and regulating excessive leukocyte trafficking. Lipoxins are transiently and locally secreted by immune cells such as neutrophils and macrophages in response to injury or inflammation. Lipoxins and epi-lipoxins bind to the high-affinity G protein-coupled lipoxin A4 (LXA4) receptor formyl peptide receptor 2 (FPR2)/ALX to resolve inflammation at nanomolar concentrations. There are several lines of evidence indicating possible anti-fibrotic properties of LXA4. It has been studied to inhibit connective tissue growth factor-induced proliferation and to interfere with TGF-β dependent pro-fibrotic properties of lung myofibroblasts. LXA4 also attenuated experimental renal fibrosis and inhibited epithelial to mesenchymal transition of renal epithelial cells in proximal tubules.

Lipoxins are well known to those of skill in the art and are described, for example, by Scalia et al. (1997) Proc. Nat. Acad. Sci. USA, ; 94(18): 9967-9972. In one illustrative, but non-limiting embodiment the lipoxin comprises LXA4 (see, e.g., FIG. 1, bottom). However, the lipoxin(s) disposed in the lipid bilayers of the presently described drug delivery vehicles are not limited to LXA4. Other lipoxins include, but are not limited to

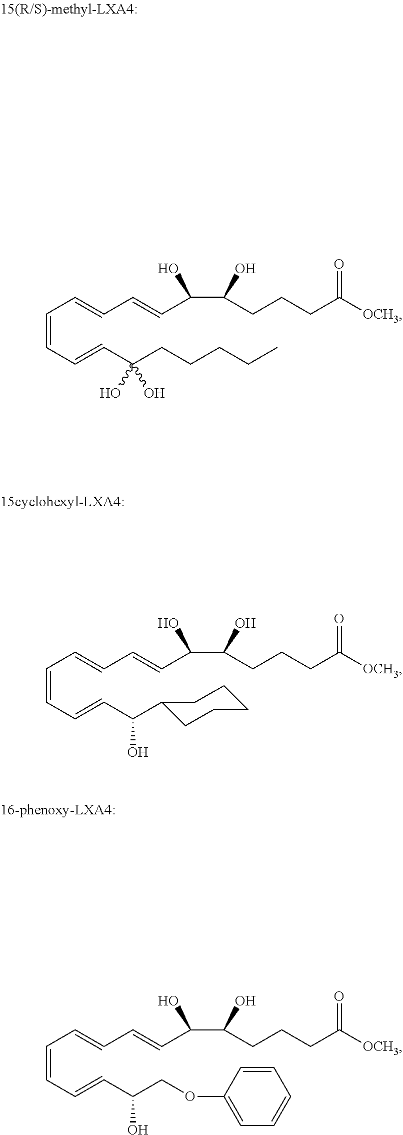

and the like.

The foregoing lipoxins are illustrative and non-limiting. Using the teaching provided herein, numerous drug delivery vehicles comprising other lipoxins disposed in a lipid bilayer will be available to one of skill in the art.

### Preparation and Remote Loading of Silicasomes, and Liposomes

In certain embodiments, the encapsulation of the chemotherapeutic agent, e.g., the ICD inducer, in the drug delivery vehicle can be optimized by using a “remote loading” strategy in which the addition of the drug (e.g., ICD-inducer such as irinotecan) to preformed liposomes or silicasomes can achieve high loading levels using a pH gradient or an ion gradient capable of generating a pH gradient (see, e.g., Ogawa et al. (2009) J. Control. Rel. 1(5): 4-10; Fritze et al. (2006) Biochimica et Biophys Acta. 1758: 1633-1640). In general, the remote loading method involves adding a cargo-trapping reagent (e.g., protonating reagent such as TEA₈SOS, ammonium sulfate, etc.) which can be added to the lipid biofilm prior to the sonication in the formation of silicasomes, or can be incorporated into the liposome prior to the formation of the liposomes e.g., as described in the Examples herein as well as in PCT Publication Nos: PCT/US2018/033265 (WO 2018/033265), and PCT/US2020/055585 (WO 2021/076630).

The cargo-trapping reagent can be selected to interact with a desired cargo (chemotherapeutic agent as described herein). In some embodiments, this interaction can be an ionic or protonation reaction, although other modes of interaction are contemplated. The cargo-trapping agent can have one or more ionic sites, i.e., can be mono-ionic or poly-ionic. The ionic moiety can be cationic, anionic, or in some cases the cargo-trapping agent can include both cationic and anionic moieties. The ionic sites can be in equilibrium with corresponding uncharged forms; for example, an anionic carboxylate (—COO⁻) can be in equilibrium with its corresponding carboxylic acid (—COOH); or in another example, an amine (—NH₂) can be in equilibrium with its corresponding protonated ammonium form (—NH₃⁺). These equilibriums are influenced by the pH of the local environment. Certain ICD-inducing weak-base reagents, such as doxorubicin, can be loaded using a trapping agent mediated approach for loading (see, e.g., PCT Publication Nos: PCT/US2018/033265 (WO 2018/033265), and PCT/US2020/055585 (WO 2021/076630) and the like).

Likewise, in certain embodiments, the cargo (chemotherapeutic agent) can include one or more ionic sites. The cargo-trapping agent and cargo can be selected to interact inside the drug delivery vehicle. This interaction can help retain the cargo within the nanoparticle until release of the cargo is desired. In some embodiments, the cargo can exist in a pH-dependent equilibrium between non-ionic and ionic forms. The non-ionic form can diffuse across the lipid bilayer and enter the liposome or the pores of the porous nanoparticle. There, the cargo-trapping agent (e.g., a polyionic cargo-trapping agent) can interact with the ionic form of the cargo and thereby retain the cargo within the nanocarrier, e.g., within the liposome or within the pores of the nanoparticle (e.g., mesoporous silica nanoparticle (MSNP)) provided the ionic forms of the cargo and cargo-trapping agent have opposite charges. In certain embodiments the interaction can be an ionic interaction and can include formation of a precipitate. Trapping of cargo within the drug delivery vehicle can provide higher levels of cargo loading compared to similar systems, e.g., nanocarriers that omit the cargo-trapping agent, or liposomes that do include a trapping agent. Release of the cargo can be achieved by an appropriate change in pH to disrupt the interaction between the cargo and cargo-trapping agent, for example, by returning the cargo to its non-ionic state which can more readily diffuse across the lipid bilayer. In one illustrative, but nonn-lembodiment, the cargo is irinotecan and the cargo-trapping agent is TEA₈SOS.

The cargo trapping agent need not be limited to TEA₈SOS. In certain embodiments the cargo trapping comprises small molecules like citric acid, (NH₄)₂SO₄, and the like. Other trapping agents include, but are not limited to, ammonium salts (e.g., ammonium sulfate, ammonium sucrose octasulfate, ammonium α-cyclodextrin sulfate, ammonium β-cyclodextrin sulfate, ammonium γ-cyclodextrin sulfate, ammonium phosphate, ammonium α-cyclodextrin phosphate, ammonium β-cyclodextrin phosphate, ammonium γ-cyclodextrin phosphate, ammonium citrate, ammonium acetate, and the like), trimethylammonium salts (e.g., trimethylammonium sulfate, trimethylammonium sucrose octasulfate, trimethylammonium α-cyclodextrin sulfate, trimethylammonium β-cyclodextrin sulfate, trimethylammonium γ-cyclodextrin sulfate, trimethylammonium phosphate, trimethylammonium α-cyclodextrin phosphate, trimethylammonium β-cyclodextrin phosphate, trimethylammonium γ-cyclodextrin phosphate, trimethylammonium citrate, trimethylammonium acetate, and the like), triethylammonium salts (e.g., triethylammonium sulfate, triethylammonium sucrose octasulfate, triethylammonium α-cyclodextrin sulfate, triethylammonium β-cyclodextrin sulfate, triethylammonium γ-cyclodextrin sulfate, triethylammonium phosphate, triethylammonium α-cyclodextrin phosphate, triethylammonium β-cyclodextrin phosphate, triethylammonium γ-cyclodextrin phosphate, triethylammonium citrate, triethylammonium acetate, and the like).

It is also worth pointing out that, in addition to TEA₈SOS, transmembrane pH gradients can also be generated by acidic buffers (e.g. citrate) (Chou et al. (2003) J. Biosci. Bioengineer., 95(4): 405-408; Nichols et al. (1976) Biochimica et Biophysica Acta (BBA)-Biomembranes, 455(1): 269-271), proton-generating dissociable salts (e.g. (NH₄)₂SO₄) (Haran et al. (1993) Biochimica et Biophysica Acta (BBA)-Biomembranes, 1151(2): 201-215; Maurer-Spurej et al. (1999) Biochimica et Biophysica Acta (BBA)-Biomembranes, 1416(1): 1-10; Fritze et al. (2006) Biochimica et Biophysica Acta (BBA)-Biomembranes, 1758(10): 1633-1640), or ionophore-mediated ion gradients from metal salts (e.g. A23187 and MnSO₄) (Messerer et al. (2004) Clinical Cancer Res. 10(19): 6638-6649; Ramsay et al. (2008) Eur. J. Pharmaceut. Biopharmaceut. 68(3): 607-617; Fenske et al. (1998) Biochimica et Biophysica Acta (BBA)-Biomembranes, 1414(1): 188-204). Moreover, it is possible to generate reverse pH gradients for drug loading, such as use a calcium acetate gradient to improve amphiphilic weak acid loading in LB-MSNP, a strategy that has been utilized in liposomes (Avnir et al. (2008) Arthritis & Rheumatism, 58(1): 119-129).

In certain embodiments the cargo-trapping reagent is particularly suitable for use with a cargo (e.g., chemotherapeutic agent described herein) that comprises an organic compound that includes at least one primary amine group, or at least one secondary amine group, or at least one tertiary amine group, or at least one quaternary amine group, or any combination thereof, capable of being protonated.

In certain embodiments the general characteristics of these cargo molecules include the following chemical properties:
(i) organic molecular compounds that include primary, secondary, tertiary or quaternary amine(s);
(ii) a pKa<11 to allow protonation and entrapment behind the LB (Zucker et al. (2009) J. Control. Release, 139(1): 73-80; Cern et al. (2012) J. Control. Release, 160(2): 147-157; Xu et al. (2014) Pharmaceut. Res. 31(10): 2583-2592);
(iii) a water solubility index of 5-25 mg/mL and amphipathic characteristics that allow diffusion across the LB;
(iv) an octanol/water partition coefficient or logP value of −3.0 to 3.0 (Zucker et al. (2009) J. Control. Release, 139(1): 73-80; Cern et al. (2012) J. Control. Release, 160(2): 147-157);
(v) suitable molecular weight with a geometric size less than MSNP pore size (2-8 nm), to allow entry into the MSNP pores (Li et al. (2012) Chem. Soc. Rev. 41(7): 2590-2605; Tang et al. (2012) Adv. Mat. 24(12): 1504-1534; Tarn et al. (2013) Acc. Chem. Res. 46(3): 792-801).

Using the teachings provided herein, numerous other chemotherapeutic agents can be remote loaded (e.g., loaded using a cargo trapping agent) into the drug delivery vehicles described herein.

Methods of making liposomes are well known to those of skill in the art and are described, for example, in PCT Publication Nos: PCT/US2018/033265 (WO 2018/033265), and PCT/US2020/055585 (WO 2021/076630).

For example, liposomes can be formed by dissolving the lipids and lipid compatible TLR7/8 agonist of lipoxin in dissolved in 5 mL chloroform in a 50 mL round bottom glass flask. The solvent can be evaporated under a rotatory vacuum to form a uniform thin lipid film that can be dried further under vacuum overnight. The film can be hydrated with a cargo-trapping agent (e.g., with 2 mL of ammonium sulfate (123 mM) and probe sonicated, e.g., for 1 h, then subsequently extruded, e.g., 15 times, through a Mini-Extruder (Avanti Polar Lipids), using, e.g., a polycarbonate membrane with 100 nm pores (Avanti Polar Lipids) at 80° C. Liposome size and morphology can be assessed by dynamic light scattering and cryoEM, respectively as desired. Unincorporated cargo-trapping agent (e.g., ammonium sulfate) can be removed, e.g., by running through a PD-10 size exclusion column. The chemotherapeutic agent to be loaded can be incubated with the above prepared liposomes, e.g., at 65° C. for 40 min. The liposomes can be fractionated across a PD-10 column, allowing the removal of free chemotherapeutic agent. Their size and morphology can be assessed by dynamic light scattering, cryoEM and UPLC/MS-MS, respectively. In another illustrative, but non-limiting embodiments, citrate can be used to load mitoxantrone.

Of course, this protocol is illustrative and non-limiting. Using this teaching, numerous other liposomes containing an ICD inducer and having a TLR7/8 agonist and/or lipoxion disposed in the lipid bilayer can be produced by one of skill in the art.

Similarly, preparation and remote-loading of a silicasome comprising a lipid compatible TLR7/8 agonist and containing a chemotherapeutic agent (e.g., an ICD-inducer) is illustrated in Example 1.

Typically preparation of silicasomes involves preparing porous nanoparticles such as MSNPs, e.g., by a sol-gel synthesis process (see. e.g., Meng et al. (2015) ACS Nano, 9(4): 540-3557). The MSNPs are then soaked in the cargo-trapping agent (e.g., ammonium sulfate) to load the agent into the pores of the MSNPs. The lipid formulation that will comprise the bilayer surrounding the silicasome is prepared, e.g., as described herein where the lipid formulation incorporates the lipid compatible TLR7/8 agonist and/or lipoxin. The cargo-trapping agent loaded MSNPs are added to the lipid film followed by sonication (e.g., 30 min probe sonication) to provide the desired silicasome. To remove the free ammonium sulfate, the particle suspension can be passed through a PD-10 size exclusion column. The pure MSNPs can be collected by centrifuging at 15,000 rpm for 15 min, three times.

This protocol also is illustrative and non-limiting. Using this teaching, numerous other silicasomes comprising a lipid compatible TLR7/8 agonist and containing a chemotherapeutic agent (e.g., ICD inducer) and various lipid formulations can be produced by one of skill in the art.

In this regard, it is noted that the lipid conjugation technology described herein can be used to make prodrugs out of chemo agents, which can be folded into a liposome. Thus, for example, ICD chemo agents like the taxanes can be incorporated into a phospholipid bilayer based on hydrophobicity, and this has been demonstrated for a MSNP where we used paclitaxel incorporation into the encapsulating phospholipid bilayer (see, e.g., Meng et al. (2015) ACS Nano, 9(4) ' 3540-3557). The same can be done for a liposome.

Thus, the versatility of the liposomal platform described herein allows the encapsulation of ICD-inducing drugs such as paclitaxel, docetaxel, doxorubicin mitroxantrone, irinotecan and etoposide through the use different loading strategies that depend on the chemical structure of the drugs. For example, it is believed that mitoxantrone, which is a weak basic molecule with MW of 444.4, water solubility of 89 mg/mL and log P value of −3.1 (mitoxantrone.www.drugbank.ca/drugs/DB01204), can be remotely loaded into a liposome or silicasome via a proton gradient, using (NH₄)₂SO₄ or citric acid. The same is possible for etoposide. Since docetaxel has high ethanol solubility (˜100 mg/mL), this lends itself to constructing liposomes by an ethanol injection method that can produce homogeneous unilamellar liposomes as described. In this method, water is poured into a concentrated lipid-ethanol solution (containing docetaxel and possibly Chol-IND in a ratiometric designed strategy), following which ethanol is removed in an evaporator (see, e.g., Pereira et al. (2016) Int. J. Pharmaceutics, 514: 150-159). Dilution with water causes spontaneous formation of small and homogenous unilamellar liposomes from the micellar aggregate. The size of the liposomes can be controlled by the ratio of ethanol to water.

These embodiments are illustrative and non-limiting. Using the teachings provided herein numerous variants will be available to one of skill in the art.

### Targeting Ligands and Immunoconjugates.

In certain embodiments the drug delivery vehicles described herein can be conjugated to one or more targeting ligands, e.g., to facilitate specific delivery in endothelial cells, to cancer cells, to fusogenic ligands, e.g., to facilitate endosomal escape, ligands to promote transport across the blood-brain barrier, and the like.

In one illustrative, but non-limiting embodiment, the drug delivery vehicle is conjugated to a fusogenic peptide such as histidine-rich HSWYG (H₂N-GLFHAIAHFIHGGWHGLIHGWYG-COOH, (SEQ ID NO:1)) (see, e.g., Midoux et al., (1998) Bioconjug. Chem. 9: 260-267).

In certain embodiments the drug delivery vehicles are conjugated to one or more targeting ligand(s) that can include antibodies as well as targeting peptides. Targeting antibodies include, but are not limited to intact immunoglobulins, immunoglobulin fragments (e.g., F(ab)′₂, Fab, etc.) single chain antibodies, diabodies, affibodies, unibodies, nanobodies, and the like. In certain embodiments antibodies will be used that specifically bind a cancer marker (e.g., a tumor associated antigen). A wide variety of cancer markers are known to those of skill in the art. The markers need not be unique to cancer cells but can also be effective where the expression of the marker is elevated in a cancer cell (as compared to normal healthy cells) or where the marker is not present at comparable levels in surrounding tissues (especially where the chimeric moiety is delivered locally).

Illustrative cancer markers include, for example, the tumor marker recognized by the ND4 monoclonal antibody. This marker is found on poorly differentiated colorectal cancer, as well as gastrointestinal neuroendocrine tumors (see, e.g., Tobi et al. (1998) Cancer Detection and Prevention, 22(2): 147-152). Other important targets for cancer immunotherapy are membrane bound complement regulatory glycoproteins CD46, CD55 and CD59, which have been found to be expressed on most tumor cells in vivo and in vitro. Human mucins (e.g., MUC1) are known tumor markers as are gp100, tyrosinase, and MAGE, which are found in melanoma. Wild-type Wilms' tumor gene WT1 is expressed at high levels not only in most of acute myelocytic, acute lymphocytic, and chronic myelocytic leukemia, but also in various types of solid tumors including lung cancer.

Acute lymphocytic leukemia has been characterized by the TAAs HLA-Dr, CD1, CD2, CD5, CD7, CD19, and CD20. Acute myelogenous leukemia has been characterized by the TAAs HLA-Dr, CD7, CD13, CD14, CD15, CD33, and CD34. Breast cancer has been characterized by the markers EGFR, HER2, MUC1, Tag-72. Various carcinomas have been characterized by the markers MUC1, TAG-72, and CEA. Chronic lymphocytic leukemia has been characterized by the markers CD3, CD19, CD20, CD21, CD25, and HLA-DR. Hairy cell leukemia has been characterized by the markers CD19, CD20, CD21, CD25. Hodgkin's disease has been characterized by the Leu-M1 marker. Various melanomas have been characterized by the HMB 45 marker. Non-hodgkins lymphomas have been characterized by the CD20, CD19, and Ia marker. And various prostate cancers have been characterized by the PSMA and SE10 markers.

In addition, many kinds of tumor cells display unusual antigens that are either inappropriate for the cell type and/or its environment or are only normally present during the organisms' development (e.g., fetal antigens). Examples of such antigens include the glycosphingolipid GD2, a disialoganglioside that is normally only expressed at a significant level on the outer surface membranes of neuronal cells, where its exposure to the immune system is limited by the blood-brain barrier. GD2 is expressed on the surfaces of a wide range of tumor cells including neuroblastoma, medulloblastomas, astrocytomas, melanomas, small-cell lung cancer, osteosarcomas and other soft tissue sarcomas. GD2 is thus a convenient tumor-specific target for immunotherapies.

Other kinds of tumor cells display cell surface receptors that are rare or absent on the surfaces of healthy cells, and which are responsible for activating cellular signaling pathways that cause the unregulated growth and division of the tumor cell. Examples include (ErbB2) HER2/neu, a constitutively active cell surface receptor that is produced at abnormally high levels on the surface of breast cancer tumor cells.

Other useful targets include, but are not limited to CD20, CD52, CD33, epidermal growth factor receptor and the like.

An illustrative, but not limiting list of suitable tumor markers is provided in Table 2. Antibodies to these and other cancer markers are known to those of skill in the art and can be obtained commercially or readily produced, e.g., using phage-display technology. Such antibodies can readily be conjugated to the drug delivery nanocarrier (e.g., LB-coated nanoparticle) described herein, e.g., in the same manner that iRGD peptide is conjugated in Example 3.

TABLE 2
Illustrative cancer markers and associated references, all of which are incorporated
herein by reference for the purpose of identifying the referenced tumor markers.

| Marker | Reference |
|---|---|
| 5 alpha reductase | Délos et al. (1998) Int J Cancer, 75: 6 840-846 |
| α-fetoprotein | Esteban et al. (1996) Tumour Biol., 17(5): 299-305 |
| AM-1 | Harada et al. (1996) Tohoku J Exp Med., 180(3): 273-288 |
| APC | Dihlmannet al. (1997) Oncol Res., 9(3) 119-127 |
| APRIL | Sordat et al. (′998) J Exp Med., 188(6): 1185-1190 |
| BAGE | Boel et al. (1995) Immunity, 2: 167-175. |
| β-catenin | Hugh et al. (1999) Int J Cancer, 82(4): 504-11 |
| Bc12 | Koty et al. (1999) Lung Cancer, 23(2): 115-127 |
| bcr-abl (b3a2) | Verfaillie et al. (′996) Blood, 87(11): 4770-4779 |
| CA-125 | Bast et al. (′998) Int J Biol Markers, 13(4): 179-187 |
| CASP-8/FLICE | Mandruzzato et al. (1997) J Exp Med., 186(5): 785-793. |
| Cathepsins | Thomssen et al. (1995) Clin Cancer Res., 1(7): 741-746 |
| CD19 | Scheuermann et al. (1995) Leuk Lymphoma, 18(5-6): 385-397 |
| CD20 | Knox et al. (1996) Clin Cancer Res., 2(3): 457-470 |
| CD21, CD23 | Shubinsky et al. (1997) Leuk Lymphoma, 25(5-6): 521-530 |
| CD22, CD38 | French et al. (1995) Br J Cancer, 71(5): 986-994 |
| CD33 | Nakase et al. (1996) Am J Clin Pathol., 105(6): 761-768 |
| CD35 | Yamakawa et al. Cancer, 73(11): 2808-2817 |
| CD44 | Naot et al. (1997) Adv Cancer Res., 71: 241-319 |
| CD45 | Buzzi et al. (1992) Cancer Res., 52(14): 4027-4035 |
| CD46 | Yamakawa et al. (1994) Cancer, 73(11): 2808-2817 |
| CD5 | Stein et al. (1991) Clin Exp Immunol., 85(3): 418-423 |
| CD52 | Ginaldi et al. (1998) Leuk Res., 22(2): 185-191 |
| CD55 | Spendlove et al. (1999) Cancer Res., 59: 2282-2286. |
| CD59 (791Tgp72) | Jarvis et al. (1997) Int J Cancer, 71(6): 1049-1055 |
| CDC27 | Wang et al. (1999) Science, 284(5418): 1351-1354 |
| CDK4 | Wölfel et al. (1995) Science, 269(5228): 1281-1284 |
| CEA | Kass et al. (1999) Cancer Res., 59(3): 676-683 |
| c-myc | Watson et al. (1991) Cancer Res., 51(15): 3996-4000 |
| Cox-2 | Tsujii et al. (1998) Cell, 93: 705-716 |
| DCC | Gotley et al. (1996) Oncogene, 13(4): 787-795 |
| DcR3 | Pitti et al. (1998) Nature, 396: 699-703 |
| E6/E7 | Steller et al. (1996) Cancer Res., 56(21): 5087-5091 |
| EGFR | Yang et al. (1999) Cancer Res., 59(6): 1236-1243. |
| EMBP | Shiina et al. (1996) Prostate, 29(3): 169-176. |
| Ena78 | Arenberg et al. (1998) J. Clin. Invest., 102: 465-472. |
| FGF8b and FGF8a | Dorkin et al. (1999) Oncogene, 18(17): 2755-2761 |
| FLK-1/KDR | Annie and Fong (1999) Cancer Res., 59: 99-106 |
| Folic Acid Receptor | Dixon et al. (1992) J Biol Chem., 267(33): 24140-72414 |
| G250 | Divgi et al. (1998) Clin Cancer Res., 4(11): 2729-2739 |
| GAGE-Family | De Backer et al. (1999) Cancer Res., 59(13): 3157-3165 |
| gastrin 17 | Watson et al. (1995) Int J Cancer, 61(2): 233-240 |
| Gastrin-releasing | Wang et al. (1996) Int J Cancer, 68(4): 528-534 |
| hormone (bombesin) | |
| GD2/GD3/GM2 | Wiesner and Sweeley (1995) Int J Cancer, 60(3): 294-299 |
| GnRH | Bahk et al. (1998) Urol Res., 26(4): 259-264 |
| GnTV | Hengstler et al. (1998) Recent Results Cancer Res., 154: 47-85 |
| gp100/Pmel17 | Wagner et al. (1997) Cancer Immunol Immunother., 44(4): |
| | 239-247 |
| gp-100-in4 | Kirkin et al. (1998) APMIS, 106(7): 665-679 |
| gp15 | Maeurer et al. (1996) Melanoma Res., 6(1): 11-24 |
| gp75/TRP-1 | Lewis et al. (1995) Semin Cancer Biol., 6(6): 321-327 |
| hCG | Hoermann et al. (1992) Cancer Res., 52(6): 1520-1524 |
| Heparanase | Vlodavsky et al. (1999) Nat Med., 5(7): 793-802 |
| Her2/neu | Lewis et al. (1995) Semin Cancer Biol., 6(6): 321-327 |
| Her3 | |
| HMTV | Kahl et al. (1991) Br J Cancer, 63(4): 534-540 |
| Hsp70 | Jaattela et al. (1998) EMBO J., 17(21): 6124-6134 |
| hTERT | Vonderheide et al. (1999) Immunity, 10: 673-679. 1999. |
| (telomerase) | |
| IGFR1 | Ellis et al. (1998) Breast Cancer Res. Treat., 52: 175-184 |
| IL-13R | Murata et al. (1997) Biochem Biophys Res Commun., 238(1): 90-94 |
| iNOS | Klotz et al. (1998) Cancer, 82(10): 1897-1903 |
| Ki 67 | Gerdes et al. (1983) Int J Cancer, 31: 13-20 |
| KIAA0205 | Guéguen et al. (1998) J Immunol., 160(12): 6188-6194 |
| K-ras, H-ras, | Abrams et al. (1996) Semin Oncol., 23(1): 118-134 |
| N-ras | |
| KSA | Zhang et al. (1998) Clin Cancer Res., 4(2): 295-302 |
| (CO17-1A) | |
| LDLR-FUT | Caruso et al. (1998) Oncol Rep., 5(4): 927-930 |
| MAGE Family | Marchand et al. (1999) Int J Cancer, 80(2): 219-230 |
| (MAGE1, | |
| MAGE3, etc.) | |
| Mammaglobin | Watson et al. (1999) Cancer Res., 59: 13 3028-3031 |
| MAP17 | Kocher et al. (1996) Am J Pathol., 149(2): 493-500 |
| Melan-A/ | Lewis and Houghton (1995) Semin Cancer Biol., 6(6): 321-327 |
| MART-1 | |
| mesothelin | Chang et al. (1996) Proc. Natl. Acad. Sci., USA, 93(1): 136-140 |
| MIC A/B | Groh et al. (1998) Science, 279: 1737-1740 |
| MT-MMP's, such as | Sato and Seiki (1996) J Biochem (Tokyo), 119(2): 209-215 |
| MMP2, MMP3, | |
| MMP7, MMP9 | |
| Mox1 | Candia et al. (1992) Development, 116(4): 1123-1136 |
| Mucin, such as | Lewis and Houghton (1995) Semin Cancer Biol., 6(6): 321-327 |
| MUC-1, MUC-2, | |
| MUC-3, and MUC-4 | |
| MUM-1 | Kirkin et al. (1998) APMIS, 106(7): 665-679 |
| NY-ESO-1 | Jager et al. (1998) J. Exp. Med., 187: 265-270 |
| Osteonectin | Graham et al. (1997) Eur J Cancer, 33(10): 1654-1660 |
| p15 | Yoshida et al. (1995) Cancer Res., 55(13): 2756-2760 |
| P170/MDR1 | Trock et al. (1997) J Natl Cancer Inst., 89(13): 917-931 |
| p53 | Roth et al. (1996) Proc. Natl. Acad. Sci., USA, 93(10): 4781-4786. |
| p97/melanotransferrin | Furukawa et al. (1989) J Exp Med., 169(2): 585-590 |
| PAI-1 | Grøndahl-Hansen et al. (1993) Cancer Res., 53(11): 2513-2521 |
| PDGF | Vassbotn et al. (1993) Mol Cell Biol., 13(7): 4066-4076 |
| Plasminogen (uPA) | Naitoh et al. (1995) Jpn J Cancer Res., 86(1): 48-56 |
| PRAME | Kirkin et al. (1998) APMIS, 106(7): 665-679 |
| Probasin | Matuo et al. (1985) Biochem Biophys Res Commun., |
| | 130(1): 293-300 |
| Progenipoietin | — |
| PSA | Sanda et al. (1999) Urology, 53(2): 260-266. |
| PSM | Kawakami et al. (1997) Cancer Res., 57(12): 2321-2324 |
| RAGE-1 | Gaugler et al. (1996) Immunogenetics, 44(5): 323-330 |
| Rb | Dosaka-Akita et al. (1997) Cancer, 79(7): 1329-1337 |
| RCAS1 | Sonoda et al. (1996) Cancer, 77(8): 1501-1509. |
| SART-1 | Kikuchi et al. (1999(Int J Cancer, 81(3): 459-466 |
| SSX gene | Gure et al. (1997) Int J Cancer, 72(6): 965-971 |
| Family | |
| STAT3 | Bromberg et al. (1999) Cell, 98(3): 295-303 |
| STn | Sandmaier et al. (1999) J Immunother., 22(1): 54-66 |
| (mucin assoc.) | |
| TAG-72 | Kuroki et al. (1990)Cancer Res., 50(16): 4872-4879 |
| TGF-α | Imanishi et al. (1989) Br J Cancer, 59(5): 761-765 |
| TGF-β | Picon et al. (1998) Cancer Epidemiol Biomarkers Prey, |
| | 7(6): 497-504 |
| Thymosin ß 15 | Bao et al. (1996) Nature Medicine. 2(12), 1322-1328 |
| IFN-α | Moradi et al. (1993) Cancer, 72(8): 2433-2440 |
| TPA | Maulard et al. (1994) Cancer, 73(2): 394-398 |
| TPI | Nishida et al. (1984) Cancer Res 44(8): 3324-9 |
| TRP-2 | Parkhurst et al. (1998) Cancer Res., 58(21) 4895-4901 |
| Tyrosinase | Kirkin et al. (1998) APMIS, 106(7): 665-679 |
| VEGF | Hyodo et al. (1998) Eur J Cancer, 34(13): 2041-2045 |
| ZAG | Sanchez et al. (1999) Science, 283(5409): 1914-1919 |
| p16INK4 | Quelle et al. (1995) Oncogene Aug. 17, 1995; 11(4): 635-645 |
| Glutathione | Hengstler (1998) et al. Recent Results Cancer Res., 154: 47-85 |
| S-transferase | |

Any of the foregoing markers can be used as targets for the targeting moieties comprising the drug delivery vehicles described herein. In certain embodiments the target markers include, but are not limited to members of the epidermal growth factor family (e.g., HER2, HER3, EGF, HER4), CD1, CD2, CD3, CD5, CD7, CD13, CD14, CD15, CD19, CD20, CD21, CD23, CD25, CD33, CD34, CD38, 5E10, CEA, HLA-DR, HM 1.24, HMB 45, 1a, Leu-M1, MUC1, PMSA, TAG-72, phosphatidyl serine antigen, and the like.

The foregoing markers are intended to be illustrative and not limiting. Other tumor associated antigens will be known to those of skill in the art.

Where the tumor marker is a cell surface receptor, a ligand to that receptor can function as targeting moieties. Similarly, mimetics of such ligands can also be used as targeting moieties. Thus, in certain embodiments peptide ligands can be used in addition to or in place of various antibodies. An illustrative, but non-limiting list of suitable targeting peptides is shown in Table 3. In certain embodiments any one or more of these peptides can be conjugated to a drug delivery vehicle described herein.

TABLE 3
Illustrative, but non-limiting peptides
that target membrane receptors expressed or
overexpressed by various cancer cells.

| Target Membrane | | SEQ |
|---|---|---|
| Receptor | Targeting Peptide | ID NO |
| Integrin | c(RGDfK) | 2 |
| receptor Aᵥβ₃ | c(RGDfC) | 3 |
| | c(RGDyC) | 4 |
| | RGD | |
| GFR | GE11 (YHWYGYTPQNVI) | 5 |
| GFR | GSG-KCCYSL | 6 |
| SSTR2 | Ostreotide | |
| GRP | QWAVGHML | 7 |
| CCK | DYMGWMDF | 8 |
| NT | RRPYIL | 9 |
| | RRPYILQLYENKPRRPYIL | 10 |
| LHRH | Gondaorelin | |
| GPRC family | Antagonist G | |
| members | | |

c() indicates cyclopeptide.
Lower case indicates “D” amino acid.

In certain embodiments the drug delivery vehicles can be conjugated to moieties that facilitate stability in circulation and/or that hide the nanocarrier from the reticuloendothelial system (REC) and/or that facilitate transport across a barrier (e.g., a stromal barrier, the blood brain barrier, etc.), and/or into a tissue. In certain embodiments the nanocarriers are conjugated to transferrin or ApoE to facilitate transport across the blood brain barrier. In certain embodiments the nanocarriers are conjugated to folate.

Methods of coupling the drug delivery vehicles to targeting (or other) agents are well known to those of skill in the art. Examples include, but are not limited to the use of biotin and avidin or streptavidin (see, e.g., U.S. Pat. No. 4,885,172 A), by traditional chemical reactions using, for example, bifunctional coupling agents such as glutaraldehyde, diimide esters, aromatic and aliphatic diisocyanates, bis-p-nitrophenyl esters of dicarboxylic acids, aromatic disulfonyl chlorides and bifunctional arylhalides such as 1,5-difluoro-2,4-dinitrobenzene; p,p′-difluoro m,m′-dinitrodiphenyl sulfone, sulfhydryl-reactive maleimides, and the like. Appropriate reactions which may be applied to such couplings are described in Williams et al. Methods in Immunology and Immunochemistry Vol. 1, Academic Press, New York 1967. In one illustrative but non-limiting approach a peptide (e.g., iRGD) is coupled to the (e.g., ICD/IDO silicasome, ICD/IDO lipid vesicle, ICD-inducing nanomaterial carrier, etc.) by substituting a lipid (e.g., DSPE-PEG₂₀₀₀) with a lipid coupled to a linker (e.g., DSPE-PEG₂₀₀₀-maleimide), allowing thiol-maleimide coupling to the cysteine-modified peptide. It will also be recognized that in certain embodiments the targeting (and other) moieties can be conjugated to other moieties comprising the lipid bilayer on a silicasome or vesicle, or comprising the nanomaterial carrier. It is also possible to improve tumor delivery of the IDO inhibitor-ICD inducing nanoparticle, (e.g., OX laden IND-Lipid bilayer-MSNP (IND-LB-MSNP), MTX loaded Chol-IND-MSNP, etc.), through co-administration (not conjugated) of the iRGD peptide to enhance particle transcytosis.

The former conjugates and coupling methods are illustrative and non-limiting. Using the teachings provided herein, numerous other moieties can be conjugated to the drug delivery vehicles described herein by any of a variety of methods.

### Pharmaceutical Formulations, Administration and Therapy

### Pharmaceutical Formulations.

In some embodiments, the drug delivery vehicles described herein are administered alone or in a mixture with a physiologically acceptable carrier (such as physiological saline or phosphate buffer) selected in accordance with the route of administration and standard pharmaceutical practice. For example, when used as an injectable, the nanocarriers can be formulated as a sterile suspension, dispersion, or emulsion with a pharmaceutically acceptable carrier. In certain embodiments normal saline can be employed as the pharmaceutically acceptable carrier. Other suitable carriers include, e.g., water, buffered water, 0.4% saline, 0.3% glycine, 5% glucose and the like, including glycoproteins for enhanced stability, such as albumin, lipoprotein, globulin, etc. In compositions comprising saline or other salt-containing carriers, the carrier is preferably added following nanocarrier formation. Thus, after the drug delivery vehicle is formed and loaded with suitable drug(s), the drug delivery vehicles described herein can be diluted into pharmaceutically acceptable carriers such as normal saline.

The pharmaceutical compositions may be sterilized by conventional, well-known sterilization techniques. The resulting aqueous solutions, suspensions, dispersions, emulsions, etc., may be packaged for use or filtered under aseptic conditions. In certain embodiments the drug delivery vehicles described herein are lyophilized, the lyophilized preparation being combined with a sterile aqueous solution prior to administration. The compositions may also contain pharmaceutically acceptable auxiliary substances as required to approximate physiological conditions, such as pH-adjusting and buffering agents, tonicity adjusting agents and the like, for example, sodium acetate, sodium lactate, sodium chloride, potassium chloride, calcium chloride, etc.

Additionally, in certain embodiments, the pharmaceutical formulation may include lipid-protective agents that protect lipids against free-radical and lipid-peroxidative damage on storage. Lipophilic free-radical quenchers, such as alpha-tocopherol and water-soluble iron-specific chelators, such as ferrioxamine, are suitable.

The concentration of the drug delivery vehicles in the pharmaceutical formulations can vary widely, e.g., from less than approximately 0.05%, usually at least approximately 2 to 5% to as much as 10 to 50%, or to 40%, or to 30% by weight and are selected primarily by fluid volumes, viscosities, etc., in accordance with the particular mode of administration selected. For example, the concentration may be increased to lower the fluid load associated with treatment. This may be particularly desirable in patients having atherosclerosis-associated congestive heart failure or severe hypertension. Alternatively, nanocarriers composed of irritating lipids may be diluted to low concentrations to lessen inflammation at the site of administration. The amount of nanocarriers administered will depend upon the particular drug used, the disease state being treated and the judgment of the clinician but will generally be between approximately 0.01 and approximately 50 mg per kilogram of body weight, preferably between approximately 0.1 and approximately 5 mg per kg of body weight.

In some embodiments, e.g., it is desirable to include polyethylene glycol (PEG)-modified phospholipids in the drug delivery vehicles described herein. Alternatively, or additionally, in certain embodiments, PEG-ceramide, or ganglioside G_{MI}-modified lipids can be incorporated in the nanocarrier (e.g., ICD/IDO silicasome, ICD/IDO lipid vesicle, ICD-inducing nanomaterial carrier, etc.). Addition of such components helps prevent nanocarrier aggregation and provides for increasing circulation lifetime and increasing the delivery of the loaded nanocarriers to the target tissues. In certain embodiments the concentration of the PEG-modified phospholipids, PEG-ceramide, or G_{MI}-modified lipids in the nanocarriers will be approximately 1 to 15%.

In some embodiments, overall drug delivery vehicle is an important determinant in nanocarrier clearance from the blood. It is believed that highly charged nanocarriers (i.e. zeta potential >+35 mV) will be typically taken up more rapidly by the reticuloendothelial system (see, e.g., Juliano (1975), Biochem. Biophys. Res. Commun. 63: 651-658 discussing liposome clearance by the RES) and thus have shorter half-lives in the bloodstream. drug delivery vehicles with prolonged circulation half-lives are typically desirable for therapeutic uses. For instance, in certain embodiments, drug delivery vehicles described herein that are maintained from 8 hrs, or 12 hrs, or 24 hrs, or greater are desirable.

In another example of their use, drug delivery vehicles described herein can be incorporated into a broad range of topical dosage forms including but not limited to gels, oils, emulsions, and the like, e.g., for the treatment of a topical cancer. For instance, in some embodiments the suspension containing the nanocarrier is formulated and administered as a topical cream, paste, ointment, gel, lotion, and the like.

In some embodiments, pharmaceutical formulations comprising the drug delivery vehicles described herein additionally incorporate a buffering agent. The buffering agent may be any pharmaceutically acceptable buffering agent. Buffer systems include, but are not limited to citrate buffers, acetate buffers, borate buffers, and phosphate buffers. Examples of buffers include, but are not limited to citric acid, sodium citrate, sodium acetate, acetic acid, sodium phosphate and phosphoric acid, sodium ascorbate, tartaric acid, maleic acid, glycine, sodium lactate, lactic acid, ascorbic acid, imidazole, sodium bicarbonate and carbonic acid, sodium succinate and succinic acid, histidine, and sodium benzoate, benzoic acid, and the like.

In some embodiments, pharmaceutical formulations comprising the drug delivery vehicles described herein additionally incorporate a chelating agent. The chelating agent may be any pharmaceutically acceptable chelating agent. Chelating agents include but are not limited to ethylene diaminetetraacetic acid (also synonymous with EDTA, edetic acid, versene acid, and sequestrene), and EDTA derivatives, such as dipotassium edetate, disodium edetate, edetate calcium disodium, sodium edetate, trisodium edetate, and potassium edetate. Other chelating agents include citric acid (e.g., citric acid monohydrate) and derivatives thereof. Derivatives of citric acid include anhydrous citric acid, trisodiumcitrate-dihydrate, and the like. Still other chelating agents include, but are not limited to, niacinamide and derivatives thereof and sodium deoxycholate and derivatives thereof.

In some embodiments, pharmaceutical formulations comprising the drug delivery vehicles described herein additionally incorporate an antioxidant. The antioxidant may be any pharmaceutically acceptable antioxidant. Antioxidants are well known to those of ordinary skill in the art and include, but are not limited to, materials such as ascorbic acid, ascorbic acid derivatives (e.g., ascorbylpalmitate, ascorbylstearate, sodium ascorbate, calcium ascorbate, etc.), butylated hydroxy anisole, buylated hydroxy toluene, alkylgallate, sodium meta-bisulfate, sodium bisulfate, sodium dithionite, sodium thioglycollic acid, sodium formaldehyde sulfoxylate, tocopherol and derivatives thereof, (d-alpha tocopherol, d-alpha tocopherol acetate, d1-alpha tocopherol acetate, d-alpha tocopherol succinate, beta tocopherol, delta tocopherol, gamma tocopherol, and d-alpha tocopherol polyoxyethylene glycol 1000 succinate) monothioglycerol, sodium sulfite and N-acetyl cysteine. In certain embodiments such materials, when present, are typically added in ranges from 0.01 to 2.0%.

In some embodiments, pharmaceutical formulations comprising the drug delivery vehicles described herein are formulated with a cryoprotectant. The cryoprotecting agent may be any pharmaceutically acceptable cryoprotecting agent. Common cryoprotecting agents include, but are not limited to, histidine, polyethylene glycol, polyvinyl pyrrolidine, lactose, sucrose, mannitol, polyols, and the like.

In some embodiments, pharmaceutical formulations comprising the drug delivery vehicles described herein are formulated with an isotonic agent. The isotonic agent can be any pharmaceutically acceptable isotonic agent. This term is used in the art interchangeably with iso-osmotic agent and is known as a compound that is added to the pharmaceutical preparation to increase the osmotic pressure, e.g., in some embodiments to that of 0.9% sodium chloride solution, which is iso-osmotic with human extracellular fluids, such as plasma. Illustrative isotonicity agents include, but are not limited to, sodium chloride, mannitol, sorbitol, lactose, dextrose and glycerol.

In certain embodiments pharmaceutical formulations of the drug delivery vehicles described herein may optionally comprise a preservative. Common preservatives include, but are not limited to, those selected from the group consisting of chlorobutanol, parabens, thimerosol, benzyl alcohol, and phenol. Suitable preservatives include but are not limited to: chlorobutanol (e.g., 0.3-0.9% w/v), parabens (e.g., 0.01-5.0%), thimerosal (e.g., 0.004-0.2%), benzyl alcohol (e.g., 0.5-5%), phenol (e.g., 0.1-1.0%), and the like.

In some embodiments, pharmaceutical formulations comprising the drug delivery vehicles described herein are formulated with a humectant, e.g., to provide a pleasant mouth feel in oral applications. Humectants known in the art include, but are not limited to, cholesterol, fatty acids, glycerin, lauric acid, magnesium stearate, pentaerythritol, and propylene glycol.

In some embodiments, an emulsifying agent is included in the formulations, for example, to ensure complete dissolution of all excipients, especially hydrophobic components such as benzyl alcohol. Many emulsifiers are known in the art, e.g., polysorbate 60.

For some embodiments related to oral administration, it may be desirable to add a pharmaceutically acceptable flavoring agent and/or sweetener. Compounds such as saccharin, glycerin, simple syrup, and sorbitol are useful as sweeteners.

### Administration

The drug delivery vehicles described herein can be administered to a subject (e.g., patient) by any of a variety of techniques.

In certain embodiments drug delivery vehicles described herein and/or pharmaceutical formulations thereof are administered parenterally, e.g., intraarticularly, intravenously, intraperitoneally, subcutaneously, or intramuscularly. In some embodiments, the pharmaceutical compositions are administered intravenously, intra-arterially, or intraperitoneally by a bolus injection (see, e.g., U.S. Pat. Nos. 3,993,754; 4,145,410; 4,235,871; 4,224,179; 4,522,803; and 4,588,578 describing administration of liposomes). Particular pharmaceutical formulations suitable for this administration are found in Remington's Pharmaceutical Sciences, Mack Publishing Company, Philadelphia, Pa., 17th ed. (1985). Typically, the formulations comprise a solution of the drug delivery nanocarrier suspended in an acceptable carrier, preferably an aqueous carrier. As noted above, suitable aqueous solutions include, but are not limited to physiologically compatible buffers such as Hanks solution, Ringer's solution, or physiological (e.g., 0.9% isotonic) saline buffer and/or in certain emulsion formulations. The solution(s) can contain formulatory agents such as suspending, stabilizing and/or dispersing agents. In certain embodiments the drug delivery vehicles described herein can be provided in powder form for constitution with a suitable vehicle, e.g., sterile pyrogen-free water, before use. For transmucosal administration, and/or for blood/brain barrier passage, penetrants appropriate to the barrier to be permeated can be used in the formulation. These compositions may be sterilized by conventional, well-known sterilization techniques, or may be sterile filtered. The resulting aqueous solutions may be packaged for use as is, or lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc., e.g., as described above.

In other methods, the pharmaceutical formulations containing the drug delivery vehicles described herein may be contacted with the target tissue by direct application of the preparation to the tissue. The application may be made by topical, “open” or “closed” procedures. By “topical” it is meant the direct application of the pharmaceutical preparation to a tissue exposed to the environment, such as the skin, oropharynx, external auditory canal, and the like. Open procedures are those procedures that include incising the skin of a patient and directly visualizing the underlying tissue to which the pharmaceutical formulations are applied. This is generally accomplished by a surgical procedure, such as a thoracotomy to access the lungs, abdominal laparotomy to access abdominal viscera, or other direct surgical approaches to the target tissue. Closed procedures are invasive procedures in which the internal target tissues are not directly visualized but accessed via inserting instruments through small wounds in the skin. For example, the preparations may be administered to the peritoneum by needle lavage. Likewise, the pharmaceutical preparations may be administered to the meninges or spinal cord by infusion during a lumbar puncture followed by appropriate positioning of the patient as commonly practiced for spinal anesthesia or metrizamide imaging of the spinal cord. Alternatively, the preparations may be administered through endoscopic devices. In certain embodiments the pharmaceutical formulations are introduced via a cannula.

In certain embodiments the pharmaceutical formulations comprising drug delivery vehicles described herein are administered via inhalation (e.g., as an aerosol). Inhalation can be a particularly effective delivery route for administration to the lungs and/or to the brain. For administration by inhalation, the drug delivery nanocarriers are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator may be formulated containing a powder mix of the compound and a suitable powder base such as lactose or starch.

In certain embodiments, the drug delivery vehicles described herein are formulated for oral administration. For oral administration, suitable formulations can be readily formulated by combining the drug delivery vehicles with pharmaceutically acceptable carriers suitable for oral delivery well known in the art. Such carriers enable the drug delivery vehicles described herein to be formulated as tablets, pills, dragees, caplets, lozenges, gelcaps, capsules, liquids, gels, syrups, slurries, suspensions and the like, for oral ingestion by a patient to be treated. For oral solid formulations such as, for example, powders, capsules and tablets, suitable excipients can include fillers such as sugars (e.g., lactose, sucrose, mannitol and sorbitol), cellulose preparations (e.g., maize starch, wheat starch, rice starch, potato starch, gelatin, gum tragacanth, methyl cellulose, hydroxypropylmethyl-cellulose, sodium carboxymethylcellulose), synthetic polymers (e.g., polyvinylpyrrolidone (PVP)), granulating agents, and binding agents. If desired, disintegrating agents may be added, such as the cross-linked polyvinylpyrrolidone, agar, or alginic acid or a salt thereof such as sodium alginate. If desired, solid dosage forms may be sugar-coated or enteric coated using standard techniques. The preparation of enteric-coated particles is disclosed for example in U.S. Pat. Nos. 4,786,505 and 4,853,230.

In various embodiments the drug delivery vehicles described herein can be formulated in rectal or vaginal compositions such as suppositories or retention enemas, e.g., containing conventional suppository bases such as cocoa butter or other glycerides. Methods of formulating active agents for rectal or vaginal delivery are well known to those of skill in the art (see, e.g., Allen (2007) Suppositories, Pharmaceutical Press) and typically involve combining the active agents with a suitable base (e.g., hydrophilic (PEG), lipophilic materials such as cocoa butter or Witepsol W45), amphiphilic materials such as Suppocire AP and polyglycolized glyceride, and the like). The base is selected and compounded for a desired melting/delivery profile.

The route of delivery of the drug delivery vehicles described herein can also affect their distribution in the body. Passive delivery of drug delivery vehicles described herein involves the use of various routes of administration e.g., parenterally, although other effective administration forms, such as intraarticular injection, inhalant mists, orally active formulations, transdermal iontophoresis, or suppositories are also envisioned. Each route produces differences in localization of the drug delivery nanocarrier.

Because dosage regimens for pharmaceutical agents are well known to medical practitioners, the amount of the drug delivery vehicles described herein that is effective or therapeutic for the treatment of a disease or condition in mammals and particularly in humans will be apparent to those skilled in the art. The optimal quantity and spacing of individual dosages of the formulations herein will be determined by the nature and extent of the condition being treated, the form, route and site of administration, and the particular patient being treated, and such optima can be determined by conventional techniques. It will also be appreciated by one of skill in the art that the optimal course of treatment, e.g., the number of doses given per day for a defined number of days, can be ascertained by those skilled in the art using conventional course of treatment determination tests.

Typically, the drug delivery vehicles described herein and/or pharmaceutical formations thereof described herein are used therapeutically in animals (including man) in the treatment of various cancers. In certain embodiments the drug delivery vehicles described herein and/or pharmaceutical formations thereof described herein are particularly well suited in conditions that require: (1) repeated administrations; and/or (2) the sustained delivery of the drug in its bioactive form; and/or (3) the decreased toxicity with suitable efficacy compared with the free drug(s) in question. In various embodiments the nanocarriers and/or pharmaceutical formations thereof are administered in a therapeutically effective dose. The term “therapeutically effective” as it pertains to the nanocarriers described herein and formulations thereof means that the combination of ICD inducer and IDO inhibitor produces a desirable effect on the cancer. Such desirable effects include but are not limited to slowing and/or stopping tumor growth and/or proliferation and/or slowing and/or stopping proliferation of metastatic cells, reduction in size and/or number of tumors, and/or elimination of tumor cells and/or metastatic cells, and/or prevention of recurrence of the cancer following remission.

Exact dosages will vary depending upon such factors as the particular composition of the drug delivery vehicle and the desirable medical effect, as well as patient factors such as age, sex, general condition, and the like. Those of skill in the art can readily take these factors into account and use them to establish effective therapeutic concentrations without resort to undue experimentation.

For administration to humans (or to non-human mammals) in the curative, remissive, retardive, or prophylactic treatment of diseases the prescribing physician will ultimately determine the appropriate dosage of the drug delivery vehicles described herein for a given human (or non-human) subject, and this can be expected to vary according to the age, weight, and response of the individual as well as the nature and severity of the patient's disease. In certain embodiments the dosage of the drug delivery vehicles described herein can be approximately equal to that employed for the free drug. However as noted above, the drug delivery vehicles described herein can significantly reduce the toxicity of the drug(s) administered thereby and significantly increase a therapeutic window. Accordingly, in some cases dosages in excess of those prescribed for the free drug(s) will be utilized.

In certain embodiments, the drug delivery vehicles described herein administered at a particular time point will be in the range from about 1 to about 1,000 mg/m²/day, or to about 800 mg/m²/day, or to about 600 mg/m²/day, or to about 400 mg/m²/day. For example, in certain embodiments a dosage (dosage regiment) is utilized that provides a range from about 1 to about 350 mg/m²/day, 1 to about 300 mg/m²/day, 1 to about 250 mg/m²/day, 1 to about 200 mg/m²/day, 1 to about 150 mg/m²/day, 1 to about 100 mg/m²/day, from about 5 to about 80 mg/m²/day, from about 5 to about 70 mg/m²/day, from about 5 to about 60 mg/m²/day, from about 5 to about 50 mg/m²/day, from about 5 to about 40 mg/m²/day, from about 5 to about 20 mg/m²/day, from about 10 to about 80 mg/m²/day, from about 10 to about 70 mg/m²/day, from about 10 to about 60 mg/m²/day, from about 10 to about 50 mg/m²/day, from about 10 to about 40 mg/m²/day, from about 10 to about 20 mg/m²/day, from about 20 to about 40 mg/m²/day, from about 20 to about 50 mg/m²/day, from about 20 to about 90 mg/m²/day, from about 30 to about 80 mg/m²/day, from about 40 to about 90 mg/m²/day, from about 40 to about 100 mg/m²/day, from about 80 to about 150 mg/m²/day, from about 80 to about 140 mg/m²/day, from about 80 to about 135 mg/m²/day, from about 80 to about 130 mg/m²/day, from about 80 to about 120 mg/m²/day, from about 85 to about 140 mg/m²/day, from about 85 to about 135 mg/m²/day, from about 85 to about 135 mg/m²/day, from about 85 to about 130 mg/m²/day, or from about 85 to about 120 mg/m²/day. In certain embodiments the dose administered at a particular time point may also be about 130 mg/m²/day, about 120 mg/m²/day, about 100 mg/m²/day, about 90 mg/m²/day, about 85 mg/m²/day, about 80 mg/m²/day, about 70 mg/m²/day, about 60 mg/m²/day, about 50 mg/m²/day, about 40 mg/m²/day, about 30 mg/m²/day, about 20 mg/m²/day, about 15 mg/m²/day, or about 10 mg/m²/day.

Dosages may also be estimated using in vivo animal models, as will be appreciated by those skill in the art.

The dose administered may be higher or lower than the dose ranges described herein, depending upon, among other factors, the bioavailability of the composition, the tolerance of the individual to adverse side effects, the mode of administration and various factors discussed above. Dosage amount and interval may be adjusted individually to provide plasma levels of the composition that are sufficient to maintain therapeutic effect, according to the judgment of the prescribing physician. Skilled artisans will be able to optimize effective local dosages without undue experimentation in view of the teaching provided herein.

Multiple doses (e.g., continuous or bolus) of the drug delivery vehicles described herein may also be administered to individuals in need thereof of the course of hours, days, weeks, or months. For example, but not limited to, 1, 2, 3, 4, 5, or 6 times daily, every other day, every 10 days, weekly, monthly, twice weekly, three times a week, twice monthly, three times a month, four times a month, five times a month, every other month, every third month, every fourth month, etc.

### Methods of Treatment.

In various embodiments methods of treatment using the drug delivery vehicles described herein and/or pharmaceutical formulation(s) comprising nanoparticle drug carriers described herein are provided. In certain embodiments the method(s) comprise a method of treating a cancer. In certain embodiments the method can comprise administering to a subject in need thereof an effective amount of the drug delivery vehicles described herein and/or a pharmaceutical formulation comprising drug delivery vehicles described herein, where the drug delivery vehicles described herein and/or said pharmaceutical formulation provide an anti-cancer drug effect, e.g., enhance a cancer-directed immunoresponse.

In certain embodiments the drug delivery vehicles described herein and/or pharmaceutical formulation is a primary therapy in a chemotherapeutic regimen. In certain embodiments the drug delivery vehicles described herein and/or pharmaceutical formulation is a component in an adjunct therapy in addition to chemotherapy using one or more other chemotherapeutic agents, and/or surgical resection of a tumor mass, and/or radiotherapy.

In certain embodiments the drug delivery vehicles described herein and/or pharmaceutical formulation is a component in a multi-drug chemotherapeutic regimen. In certain embodiments the multi-drug chemotherapeutic regimen comprises at least two drugs selected from the group consisting of irinotecan (IRIN), oxaliplatin (OX), 5-fluorouracil (5-FU), and leucovorin (LV). In certain embodiments the multi-drug chemotherapeutic regimen comprises at least three drugs selected from the group consisting of irinotecan (IRIN), oxaliplatin (OX), 5-fluorouracil (5-FU), and leucovorin (LV). In certain embodiments the multi-drug chemotherapeutic regimen comprises at least irinotecan (IRIN), oxaliplatin (OX), 5-fluorouracil (5-FU), and leucovorin (LV).

In various embodiments the drug delivery vehicles described herein and/or pharmaceutical formulation(s) thereof described herein are effective for treating any of a variety of cancers. In certain embodiments the cancer is pancreatic ductal adenocarcinoma (PDAC). In certain embodiments the cancer is a cancer selected from the group consisting of acute lymphoblastic leukemia (ALL), acute myeloid leukemia (AML), adrenocortical carcinoma, AIDS-related cancers (e.g., Kaposi sarcoma, lymphoma), anal cancer, appendix cancer, astrocytomas, atypical teratoid/rhabdoid tumor, bile duct cancer, extrahepatic cancer, bladder cancer, bone cancer (e.g., Ewing sarcoma, osteosarcoma, malignant fibrous histiocytoma), brain stem glioma, brain tumors (e.g., astrocytomas, glioblastoma, brain and spinal cord tumors, brain stem glioma, central nervous system atypical teratoid/rhabdoid tumor, central nervous system embryonal tumors, central nervous system germ cell tumors, craniopharyngioma, ependymoma, breast cancer, bronchial tumors, burkitt lymphoma, carcinoid tumors (e.g., childhood, gastrointestinal), cardiac tumors, cervical cancer, chordoma, chronic lymphocytic leukemia (CLL), chronic myelogenous leukemia (CML), chronic myeloproliferative disorders, colon cancer, colorectal cancer, craniopharyngioma, cutaneous t-cell lymphoma, duct cancers e.g. (bile, extrahepatic), ductal carcinoma in situ (DCIS), embryonal tumors, endometrial cancer, ependymoma, esophageal cancer, esthesioneuroblastoma, extracranial germ cell tumor, extragonadal germ cell tumor, extrahepatic bile duct cancer, eye cancer (e.g., intraocular melanoma, retinoblastoma), fibrous histiocytoma of bone, malignant, and osteosarcoma, gallbladder cancer, gastric (stomach) cancer, gastrointestinal carcinoid tumor, gastrointestinal stromal tumors (GIST), germ cell tumors (e.g., ovarian cancer, testicular cancer, extracranial cancers, extragonadal cancers, central nervous system), gestational trophoblastic tumor, brain stem cancer, hairy cell leukemia, head and neck cancer, heart cancer, hepatocellular (liver) cancer, histiocytosis, langerhans cell cancer, Hodgkin lymphoma, hypopharyngeal cancer, intraocular melanoma, islet cell tumors, pancreatic neuroendocrine tumors, kaposi sarcoma, kidney cancer (e.g., renal cell, Wilm's tumor, and other kidney tumors), langerhans cell histiocytosis, laryngeal cancer, leukemia, acute lymphoblastic (ALL), acute myeloid (AML), chronic lymphocytic (CLL), chronic myelogenous (CML), hairy cell, lip and oral cavity cancer, liver cancer (primary), lobular carcinoma in situ (LCIS), lung cancer (e.g., childhood, non-small cell, small cell), lymphoma (e.g., AIDS-related, Burkitt (e.g., non-Hodgkin lymphoma), cutaneous T-Cell (e.g., mycosis fungoides, Sézary syndrome), Hodgkin, non-Hodgkin, primary central nervous system (CNS)), macroglobulinemia, Waldenström, male breast cancer, malignant fibrous histiocytoma of bone and osteosarcoma, melanoma (e.g., childhood, intraocular (eye)), merkel cell carcinoma, mesothelioma, metastatic squamous neck cancer, midline tract carcinoma, mouth cancer, multiple endocrine neoplasia syndromes, multiple myeloma/plasma cell neoplasm, mycosis fungoides, myelodysplastic syndromes, chronic myeloid leukemia (CML), multiple myeloma, nasal cavity and paranasal sinus cancer, nasopharyngeal cancer, neuroblastoma, oral cavity cancer, lip and oropharyngeal cancer, osteosarcoma, ovarian cancer, pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, plasma cell neoplasm, pleuropulmonary blastoma, primary central nervous system (CNS) lymphoma, prostate cancer, rectal cancer, renal cell (kidney) cancer, renal pelvis and ureter, transitional cell cancer, rhabdomyosarcoma, salivary gland cancer, sarcoma (e.g., Ewing, Kaposi, osteosarcoma, rhadomyosarcoma, soft tissue, uterine), Sézary syndrome, skin cancer (e.g., melanoma, merkel cell carcinoma, basal cell carcinoma, nonmelanoma), small intestine cancer, squamous cell carcinoma, squamous neck cancer with occult primary, stomach (gastric) cancer, testicular cancer, throat cancer, thymoma and thymic carcinoma, thyroid cancer, trophoblastic tumor, ureter and renal pelvis cancer, urethral cancer, uterine cancer, endometrial cancer, uterine sarcoma, vaginal cancer, vulvar cancer, Waldenström macroglobulinemia, and Wilm's tumor.

In certain embodiments the drug delivery vehicles described herein is not conjugated to an iRGD peptide and the nanocarrier is administered in conjunction with an iRGD peptide (e.g., the nanocarrier and the iRGD peptide are co-administered as separate formulations).

In various embodiments of these treatment methods, drug delivery vehicles described herein and/or pharmaceutical formulation is administered via a route selected from the group consisting of intravenous administration, intraarterial administration, intracerebral administration, intrathecal administration, oral administration, aerosol administration, administration via inhalation (including intranasal and intratracheal delivery, intracranial administration via a cannula, and subcutaneous or intramuscular depot deposition. In certain embodiments the nanocarrier and/or pharmaceutical formulation is administered as an injection, from an IV drip bag, or via a drug-delivery cannula. In various embodiments the subject is a human and in other embodiments the subject is a non-human mammal.

### Kits.

In certain embodiments, kits are provided containing reagents for the practice of any of the methods described herein. In certain embodiments the kit comprises a container containing a drug delivery vehicle as described herein (e.g., a liposome or a silicasome).

Additionally, in certain embodiments, the kits can include instructional materials disclosing the means of the use of the drug delivery vehicles described herein for a cancer (e.g., a pancreatic cancer, gastric cancer, cervical cancer, ovarian cancer, etc.).

In addition, the kits optionally include labeling and/or instructional materials providing directions (e.g., protocols) for the use of the materials described herein, e.g., alone or in combination for the treatment of various cancers. Instructional materials can also include recommended dosages, description(s) of counterindications, and the like.

While the instructional materials in the various kits typically comprise written or printed materials they are not limited to such. Any medium capable of storing such instructions and communicating them to an end user is contemplated by this invention. Such media include but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### Development of a Dual Delivery TLR7/8 Agonist-Chemotherapeutic Drug (e.g., ICD Inducer) Delivery Vehicle

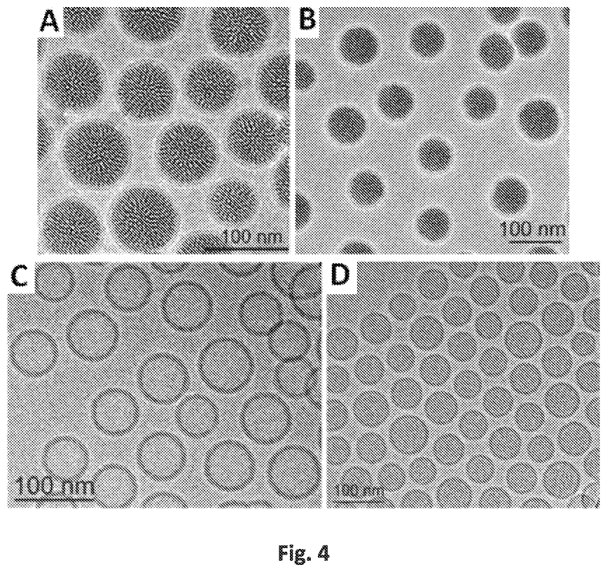

FIG. 2, panels A-B illustrates encapsulation of the TLR7/8 agonist, 3M-052, in liposomes and silicasomes. In particular FIG. 2, panel A depicts loading of lipophilic TLR7/8 agonist, 3M-052, into a lipid bilayer structure that could be applied to liposomes and silicasomes. FIG. 2, panel B depicts the synthesis of 3M-Liposome-IRIN. In brief, lipids and 3M-052 were dissolved in chloroform, at a molar ratio of 55.5:38.5:2.73.3 for DSPC, cholesterol, DSPE-PEG₂ₖ and 3M-052. The solvent was evaporated to the formation of a thin lipid film. The dried film was rehydrated in ammonium sulfate solution (pH 5.4), followed by an extrusion through a series of filters. For creation of a cross-bilayer proton gradient, to allow remote drug loading, the buffer in which the 3M-Liposomes were suspended was changed to a HEPES-buffered dextrose solution (5 mM HEPES, 5% dextrose, pH 6.5) by a de-salting, size-exclusion PD-10 column. For IRIN remote loading, 3M-Liposomes were mixed with IRIN and incubated at 65° C. for 1 h. After cooling down at 4° C. for 0.5 h, 3M-Liposome-IRIN was purified by column PD-10. The liposomes were characterized for size and zeta potential and the drug loading capacity was determined by a ZETAPALS instrument (Brookhaven Instruments Corp.) and UV spectroscopy. We also performed cryo EM to determine liposome structure, as shown in FIG. 4, panels C and D.

Tables 4-6 illustrate the characterization of 3M-liposome and 3M-liposome-IRIN (Tables 4 and 6), and 3M-Silicasome and 3M-Silicasome-IRIN (Table 5).

TABLE 4
Characterization of 3M-Liposome and 3M-Liposome-
IRIN for hydrodynamic size, polydispersity Index
(PDI), zeta potential and drug loading capacities.

| | | | | 3M-052 | IRIN |
|---|---|---|---|---|---|
| | | | Zeta | loading | loading |
| | Size | | potential | capacity | capacity |
| Vehicle | (nm) | PDI | (mV) | (%) | (%) |
| 3M-Liposome | 135.8 ± 2.2 | 0.06 | −14 ± 2.23 | 1.5 | — |
| 3M-Liposome- | 150.5 ± 3.4 | 0.033 | −37.76 ± 4.56 | 1.5 | 28.6 |
| IRIN | | | | | |

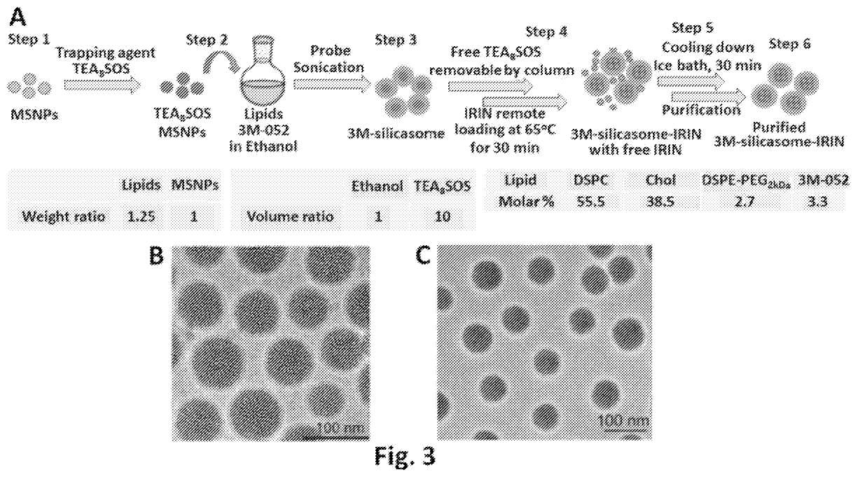

FIG. 3 shows the synthesis and preparation of the 3M-Silicasome-IRIN. Briefly, the mixture of lipids and 3M-052 dissolved in ethanol (500 mg/mL) were incubated at 65° C. before mesoporous silica nanoparticles (MSNPs), bathed in the trapping agent TEA₈SOS solution (40 mg/mL), were added to the solution at a volume ratio of 1:10. This yields mixing of lipids with the 3M-052:MSNPs in a ratio of 1.25:1 (w/w). Probe sonication was then used for 15 min with 15/15 s on/off working cycle, at a power output of 32.5 W. Following the removal of free TEA₈SOS during column exchange in a HEPES-buffered dextrose solution (5 mM HEPES, 5% dextrose, pH 6.5), IRIN was remotely loaded into purified 3M-silicasomes at 65° C. for 30 min. After cooling down on ice bath for another 30 min and centrifugation, purified 3M-Silicasome-IRIN was obtained. The silicasomes were characterized for size and zeta potential and the drug loading capacity was determined by a ZETAPALS instrument (Brookhaven Instruments Corporation), UV spectroscopy and TGA.

We also performed cryo EM to determine liposome structure, as shown in FIG. 4, panel B. As a control, we also synthesized a silicasome that incorporates 3M-052, without Irinotecan loading (FIG. 4, panel A). In addition to the use of a lipid biofilm technique for construction of liposomes and silicasomes, it is also important to point out that larger scale production of the same carriers can be achieved by the use of flow-through sonication (for silicasomes) or the use of a microfluidic synthesizer, which blends and aqueous with a lipid solvent stream, with active mixing.

TABLE 5
Characterization of 3M-Liposome and 3M-Liposome-IRIN for hydrodynamic size,
polydispersity Index (PDI), zeta potential and drug loading capacities.

| | | | | 3M-052 | IRIN |
|---|---|---|---|---|---|
| | | | Zeta | loading | loading |
| | Size | | potential | capacity | capacity |
| Vehicle | (nm) | PDI | (mV) | (%) | (%) |
| 3M-Silicasome | 112.2 ± 1.0 | 0.102 | −9.09 ± 0.91 | 2.1 | — |
| 3M-Silicasome- | 127.0 ± 2.9 | 0.066 | −12.84 ± 1.99 | 2.1 | 37.9 |
| IRIN | | | | | |

TABLE 6
Characterization of 3M-Liposome and 3M-Liposome-
IRIN for hydrodynamic size, polydispersity Index
(PDI), zeta potential and drug loading capacities.

| | | | | 3M-052 | IRIN |
|---|---|---|---|---|---|
| | | | Zeta | loading | loading |
| | Size | | potential | capacity | capacity |
| Vehicle | (nm) | PDI | (mV) | (%) | (%) |
| 3M-Liposome | 89.6 ± 1.1 | 0.040 | −7.83 ± 1.99 | 2.4 | 0 |
| 3M-Liposome- | 97.5 ± 0.2 | 0.078 | −15.31 ± 1.4 | 2.4 | 22.2 |
| IRIN | | | | | |

FIG. 4, panels A-D, shows example cryo-electron images of silicasomes and liposomes. A cryoEM microscope (TF20 FEI TecnaiG2) was used to assess the morphology, uniformity and integrity of the lipid bilayer coating. FIG. 4, panel A depicts the 3M-Silicasome, FIG. 4, panel B shows the 3M-Silicasome-IRIN, FIG. 4, panel C shows the liposome structure and FIG. 4, panel D, shows the 3M-Liposome-IRIN.

The images show uniform particle sizes, in which the surfaces of the 3M-Silicasome and 3M-Silicasome-IRIN were completely covered by a 7 nm thick lipid bilayer. The particle sizes were ˜88 nm, with negative zeta potential. The images of the liposomes show unilamellar bilayer structures and the sizes were ˜60 nm, with negative zeta potential.

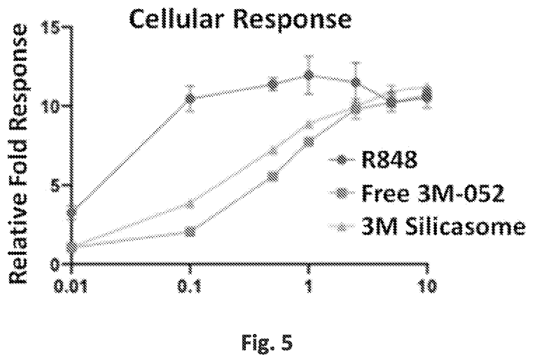

FIG. 5 shows a demonstration of a TLR7 agonist impact on HEK-Blue™ mTLR7 cells, which express both a copy of the murine TLR7 gene and an NF-κB/AP-1-inducible SEAP (secreted embryonic alkaline phosphatase) reporter gene. TLR7 agonist activity was assessed by comparing the effect of the 3M-Silicasome on the SEAP levels in comparison to free R848 and free 3M-052, over a concentration range from 0.01 to 10 μm for 20 h. The signal was detected by the HEK-Blue™ Detection kit.

The data showed that both free 3M-052 and the 3M-Silicasome could induce similar fold increases in the SEAP levels in HEK-Blue™ mTLR7 cells. Both stimuli had comparable dose-response kinetics, which were not as abundant as the positive control (R848). These data show effective 3M-053 cellular delivery from the 3M-Silicasome.

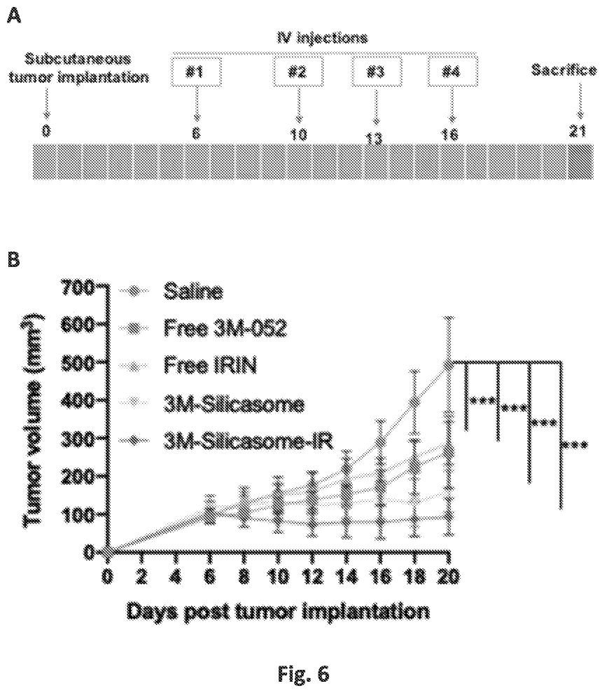

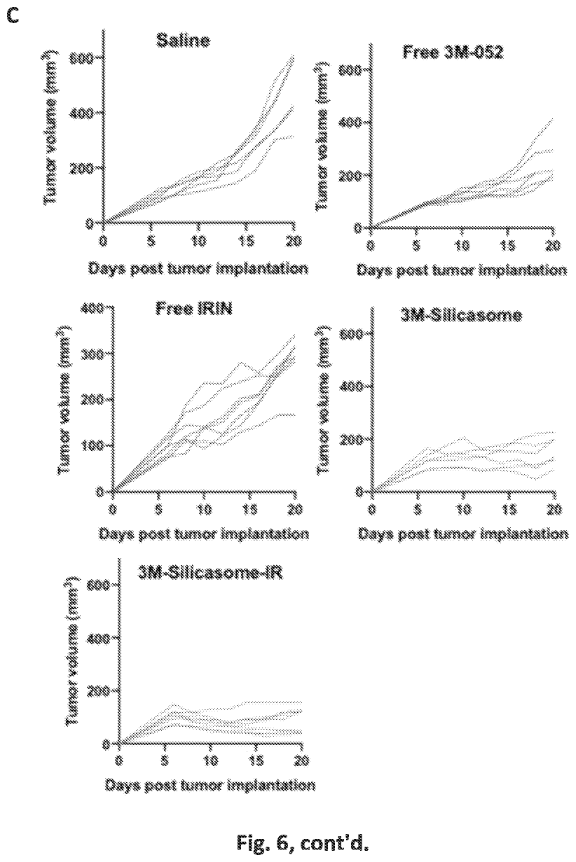

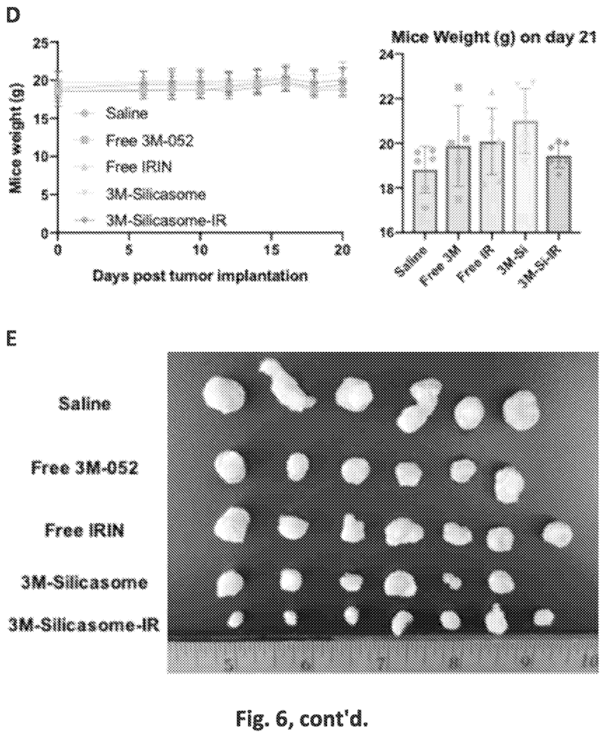

FIG. 6 illustrates the therapeutic and synergistic drug effects of the dual delivery silicasome in a subcutaneous KPC pancreatic cancer model. The schematic in FIG. 6, panel A depicts the treatment outline for conducting the subcutaneous KPC tumor experiment. In brief, 100 μL of PBS/Matrigel (1:1 v/v), containing 1×10⁶ KPC cells were subcutaneously injected into the right flank of female B6129SF1/J mice (8-10 weeks) 6 days before the onset of treatment. Tumor-bearing mice were randomly divided into 5 groups (n=6 or 7), which received IV administration of the different treatment modalities, every 3-4 days on 4 occasions. The different treatments were: saline, free 3M-052, free IRIN, 3M-Silicasome and 3M-Silicasome-IR. IRIN was administered at 40 mg/kg while the 3M-052 dose was delivered at 2 mg/kg. FIG. 6, panel B, depicts the average subcutaneous KPC tumor growth kinetics, as determined by tumor volume. Tumor size was measured every 2 days, and tumor volume was calculated by the formula: tumor volume (mm³)=(length in mm)×(width in mm)²/2. FIG. 6, panel C depicts the spaghetti growth curves (tumor volume) for each animal in each of the treatment groups, while FIG. 6, panel D, r reports animal body weight over 20 days. FIG. 6, panel E, depicts photographic images of tumors in each treatment group on day 21.

The results depicting tumor volume and spaghetti plots show that while the tumors in control animals (saline) continued to grow over time, the various different treatments had different impacts. These results demonstrate that, while free 3M-052 and IRIN had modest effects, there was a stronger response in response to the 3M-slicasome. However, the best outcome was achieved during the administration of the 3M-silicasome-IR. These results demonstrate the efficacy of 3M-052 delivery by the silicasome, which significantly increased during Irinotecan co-delivery by the same silicasome. This shows the synergistic effect of dual drug delivery by the silicasome, which indicates that TLR7 acting synergistically with the immunogenic cell death response (ICD) induced by IRIN. Noteworthy, there was no weight loss in any of the groups, which is in favor of our previous studies showing the safety of Irinotecan delivery by silicasomes.

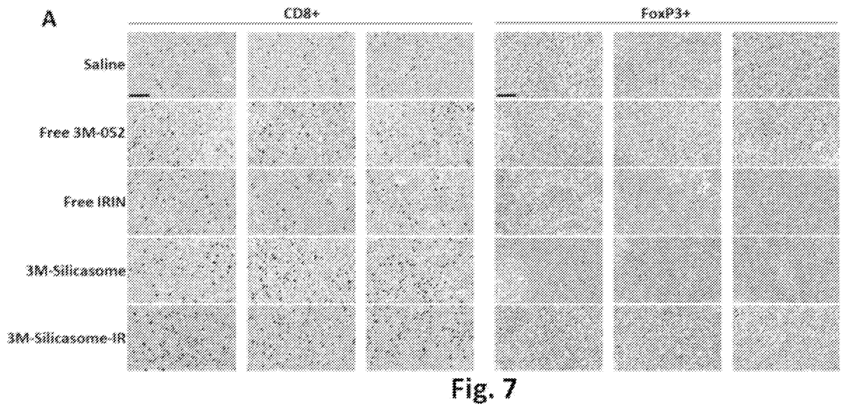

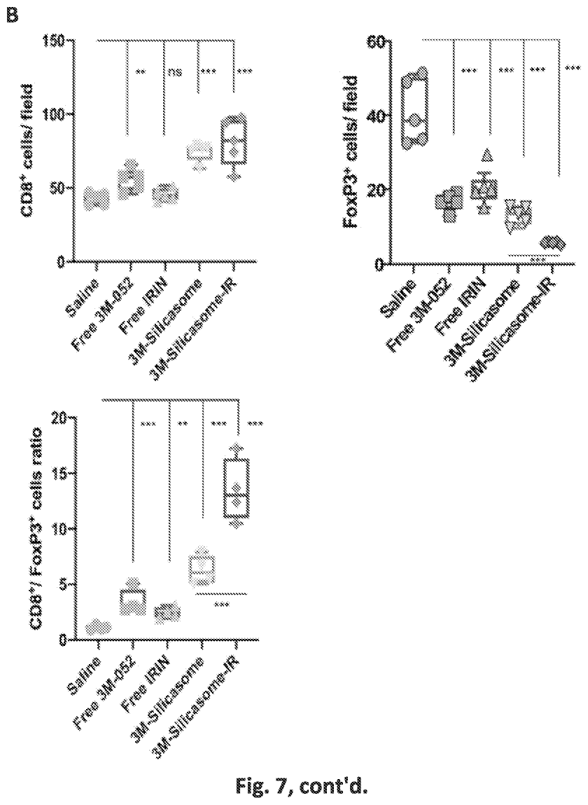

The tumors harvested from the animals are undergoing immunohistology to characterize the specifics of the immune response, including biomarkers that are indicative of the generation of cytotoxic T-cells, reprogramming of macrophage activity, inflammation markers and activation of TLR signaling pathways. The Immunohistochemistry analysis in FIG. 7 demonstrates the presence and distribution of CD8+ cytotoxic T cells and FoxP3+ regulatory T cells (Treg) and the subcutaneous tumor sites. FIG. 7, panel A depicts the representative IHC images on day 21. Bar is 100 μm. Basically, these images show increased recruitment of cytotoxic T-cells in response to monotherapy with Irinotecan or 3M-052, which was increased synergistically by combination therapy. The opposite trend was seen for disappearance of Tregs. The slides were used for quantitative image analysis of cell number. FIG. 7, panel B, depicts the quantitative data for CD8+ and FoxP3+ cells, as well as the ratio of the cell types. The statistics indicate a significant increase in the number of CD8⁺ T-cells with all treatments, but the higher statistical significance for encapsulated 3M-052, with or without IRIN. In contrast, there is a highly significant decrease in the number of Tregs with all treatments, with encapsulated 3M-052 providing the biggest decline. Moreover, in this case, the dual delivery carrier induced a significant difference from the 3M-052 only carrier, indicative of a synergistic effect. Plotting of the CD8/Treg ratio demonstrated a significant increase with all treatments. However, the highest ratio was achieved with encapsulated 3M-052, in which the dual delivery carrier was significantly higher than the 3M-052 only carrier. This confirms a synergistic effect.

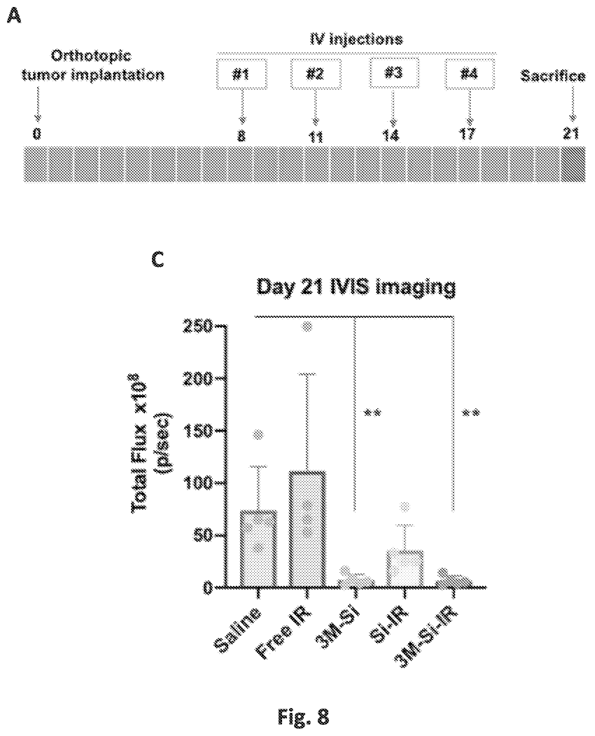

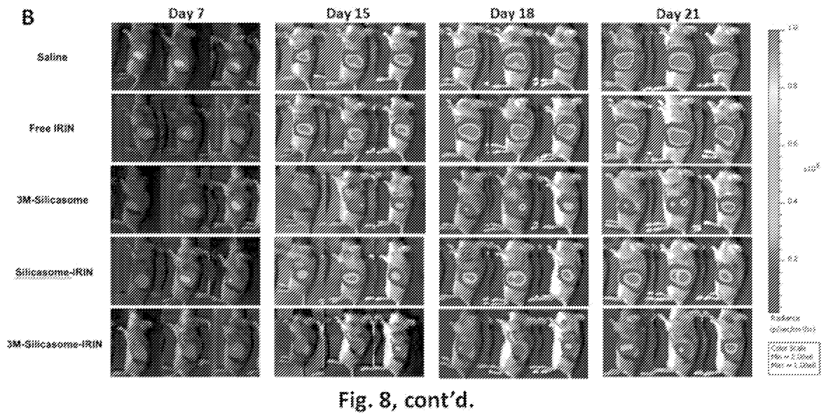

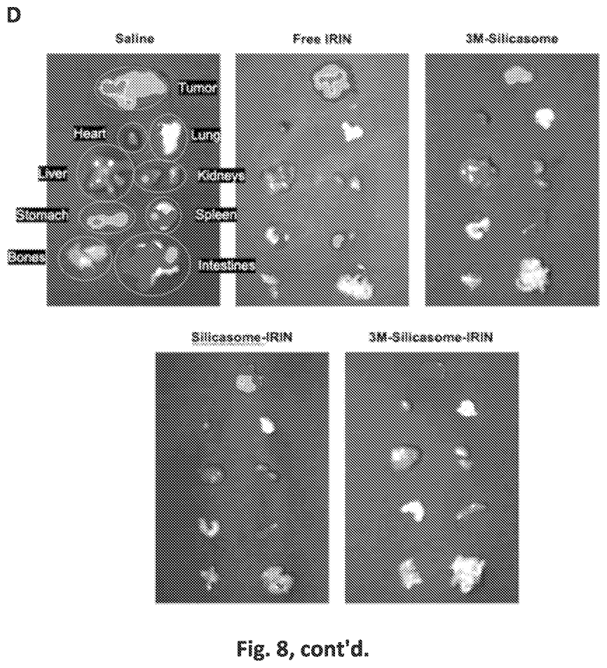

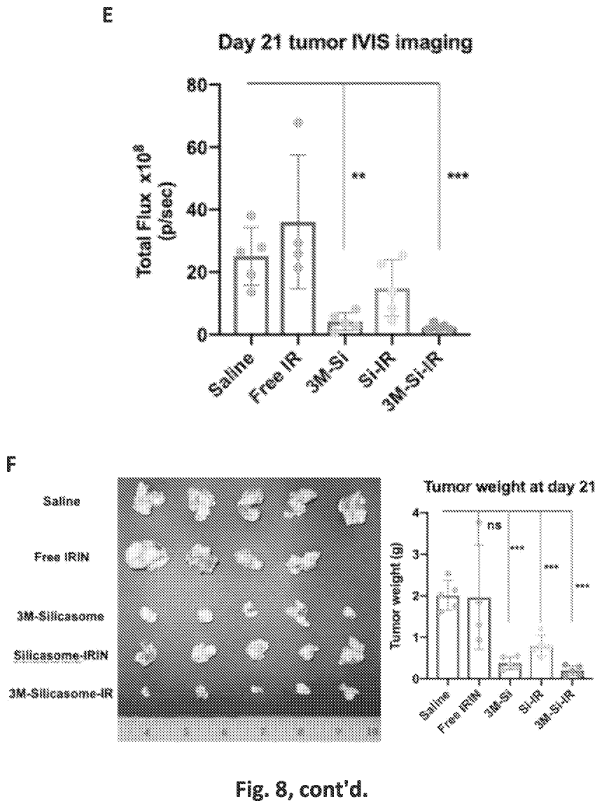

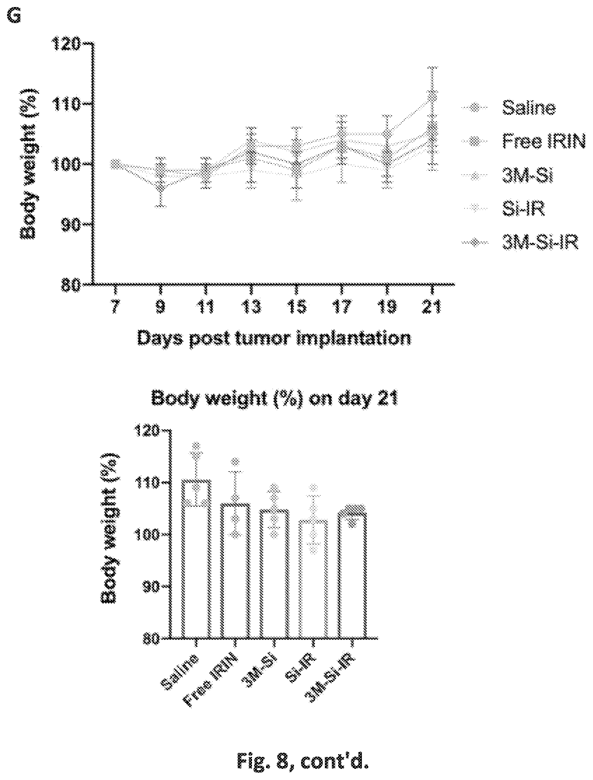

FIG. 8 illustrates a study of the therapeutic and synergistic drug effects of the dual delivery silicasome in an orthotopic KPC pancreatic cancer model. FIG. 8, panel A, depicts the experimental outline of the study conducted by orthotopic implantation KPC-luc tumor cells in the pancreatic tail of syngeneic mice. In brief, 50 μL of PBS/Matrigel (6:4 v/v), containing 0.8×10⁶ KPC-luc cells was orthotopically injected into the tail of the pancreas of female B6129SF1/J mice 8 days before the onset of treatment. Once the spending tumor growth was confirmed by IVIS bioluminescence imaging 1 day prior to drug injection, tumor-bearing mice were randomly divided into 5 groups (n=5), namely: saline, free IRIN, 3M-Silicasome, Silicasome-IRIN and 3M-Silicasome-IR (IRIN and 3M-052. These animals received IV injection of the different treatment modalities every 3 days on 4 occasions. The differently dose equivalents for IRIN and 3M-052 amounted to 40 mg/kg and 2 mg/kg respectively. FIG. 8, panel B depicts the IVIS imaging intensity for each orthotopic tumor in each of the animal groups, recorded on days 7, 15, 18 and 21. FIG. 8, panel C depicts the average quantitative tumor bioluminescence in each group at day 21, as calculated by IVIS software. **p<0.01.

Mice were sacrificed on day 21. FIG. 8, panel D, depicts the IVIS imaging performed on the ex vivo explanted tumor tissues and potential metastatic organs on day 21. The average quantitative bioluminescence of the primary tumors in each group is displayed in FIG. 8, panel E. FIG. 8, panel F, depicts photographic images and primary tumor weights in each group shown, at the same level of magnification, on day 21. FIG. 8, panel G depicts animal body weights over a time period of 21 days.

The results demonstrate the efficacy of 3M-052 delivery by the silicasome, either as a single treatment or in combination with Irinotecan. Prior to the onset of treatment (day 7), each animal displayed roughly the same level of tumor bioluminescence. The tumors in the saline group (control) group continued to grow rapidly, achieving highest bioluminescence intensity at day 21. Mice in the free IRIN group had similar IVIS imaging effects as the control group, indicating that the free drug had little effect on orthotopic tumors. This stands in contrast to treatment with the 3M-Silicasome, Silicasome-IRIN and 3M-Silicasome-IR groups, which showed a significant reduction in bioluminescence intensity than saline or free IRIN groups. These results are also in agreement with significant reduction in tumor weight during treatment with each of these carriers. However, comparison of the carriers demonstrates that the treatment effect of the 3M-Silicasome or the 3M-Silicasome-IR was significantly better than the Silicasome-IRIN, further emphasizing the efficacy of encapsulated 3M-052 delivery. While the dual delivery response was slightly more effective than the response to the 3M-Silicasome, the additional impact did not reach statistical significance.

The ex vivo IVIS imaging data demonstrates extensive metastasis of the primary tumor to surrounding organs (kidneys, liver, stomach, spleen and intestines) in the control group. While the same metastatic burden could be observed in the free IRIN group, there were barely any metastases present in the 3M-Silicasome, Silicasome-IRIN and 3M-Silicasome-IR treatment groups. In addition, the quantitative ex vivo IVIS imaging results could further substantiate the significant inhibition of the primary orthotopic tumor growth in all the encapsulated treatment groups. We did not observe a significant reduction in animal weight in all of the treatment groups, which is in keeping with the treatment safety of the silicasomes. As for the subcutaneous model, the major hypothesis for the treatment effect is TLR7 immune activation as well as the contribution by immunogenic cell death. The excised tumor tissues are currently being studied by Immunohistochemistry.

Tumors were harvested from the animals for immunohistology to characterize the specifics of the immune response, including biomarkers that are indicative of the generation of cytotoxic T-cells, reprogramming of macrophage activity, inflammation markers and activation of TLR signaling pathways. These studies are ongoing. We have also performed pharmacokinetic analysis, which is ongoing.

Over the past decade, advances in diagnostic approaches, perioperative management, radiotherapy techniques, and systemic therapies for advanced disease have made relevant but only modest incremental progress in patient outcomes. For the purpose of reducing toxicity of irinotecan, encapsulated drug delivery has been developed. The liposomal carrier ONIVYDE® was approved in 2015 for combination with 5-FU/LV as a second line therapeutic option for patients with metastatic PDAC who progressed after gemcitabine monotherapy. Due to the aggressiveness of this disease and the dearth of effective therapies, the majority of patients receive only a single line of chemotherapy.

While we have demonstrated that we can improve over ONIVYDE® efficacy and safety, we have recently also provided evidence that Irinotecan delivery by the silicasome induce a more effective immunogenic response than the liposome (see, e.g., Liu et al. (2021) Adv. Sci. 8(6): 2002147). This was corroborated by augmenting the Irinotecan response by anti-PD1 therapy. We now also demonstrate a strong response to the encapsulated delivery of a TLR7 agonist, which shows synergy with Irinotecan in the subcutaneous model. These results have commercial implications for considering the use of encapsulated 3M-052 to boost the immunogenic cell death effects, as well as using encapsulated 3M-052 monotherapy to enhance the response to other immunomodulatory agents, including chemotherapy that induce immunogenic cell death.

Based on the success of dual drug delivery demonstrated in these studies, a number of additional commercial applications can be envisaged. First, we can develop a geo-delivery liposome for Irinotecan plus 3M-052. Second, we can develop liposomes and silicasomes that deliver 3M-052 plus any of a number of ICD inducing chemo agents that can be remotely loaded, including, for example, Irinotecan, doxorubicin, and mitoxantrone. Third, both lipid bilayer encapsulated carriers can be used for combining 3M-052 with other small molecule agent that can be remote loaded, including GSK3 inhibitors and CXCR4 inhibitors. Fourth, 3M-025 can be incorporated into lipid bilayer carriers together with additional drugs that can be incorporated into the lipid bilayer, such as TLR4 agonists, IDO-1 inhibitors, small molecule PD1 inhibitors, etc. All of the above could also be implemented in the treatment of cancers, other than PDAC, including triple negative breast cancer, lung cancer, colon cancer, renal cancer, etc.

### Example 2

### Development of a Dual Delivery Lipoxin-Chemotherapeutic Drug (e.g., ICD Inducer) Delivery Vehicle

Pancreatic ductal adenocarcinoma (PDAC) is highly aggressive form of cancer with an estimated 5-year survival rate of less than 10%. It's the fourth leading cause of cancer-related deaths in the United States owing to the limited success in available treatment modalities.

Pancreatic tumors are characterized by a hypovascular, desmoplastic stroma that results in poor drug delivery to extravascular tumor tissue. In addition, its immune excluded—microenvironment prevents generation of neoantigens to promote a sufficiently strong antigen specific immune response, leading to aggressive growth and metastatic spread. The American Cancer Society projects about 62,210 new cases of pancreatic cancer and about 49,830 deaths in the United States in 2022.

Irinotecan, a topoisomerase I inhibitor, is used against PDAC in combination regimens, most notably FOLFIRINOX (5-FU, folinic acid, irinotecan, and oxaliplatin) and have shown remarkable improvement in overall survival in comparison to gemcitabine monotherapy. Despite its efficacy, the clinical application of irinotecan is dose-limited due to fatal side effects including intestinal toxicity, electrolyte imbalance and dehydration. Introduction of liposomal carriers for targeted delivery of irinotecan such as Onivyde has led to improved survival in PDAC patients and reduction in drug toxicity. Recent studies with irinotecan nanocarriers by our group have demonstrated its potential to induce immunogenic cell death (ICD), a promising endogenous vaccination approach for cancer immunotherapy.

PDAC consists of transformed cells, immune cells as well as a non-transformed stroma that accounts for 70-90% of tumor mass. The tumor microenvironment is a crucial factor in the pathobiology and progression of PDAC. Pancreatic stellate cells (PSCs) are myofibroblast-like cells in the pancreas that interact with transformed cells and mount a dysregulated wound healing response. The resulting fibrosis progresses to generate the desmoplastic stroma which in turn modulates immune evasion, proliferation, EMT, migration and invasion of pancreatic cancer cells. PSCs are activated by a plethora of molecules including transforming growth factor-β (TGF-β) which is released by cancer cells and immune cells. Upon activation, PSCs secrete cytokines such as IL6, IL1 p and TGF-β as paracrine signals to cancer cells to sustain proliferation, migration, and invasiveness. PSCs are not passive bystanders but pro-inflammatory, tumor-supporting and therefore, warrant therapeutic targeting alongside tumor cells to establish treatment longevity and improve patient outcome.

Lipoxins belong to the first recognized class of anti-inflammatory lipids that function as endogenous “stop signals”, impeding the deleterious responses of PMNs and regulating excessive leukocyte trafficking. Lipoxins are transiently and locally secreted by immune cells such as neutrophils and macrophages in response to injury or inflammation. Lipoxins and epi-lipoxins bind to the high-affinity G protein-coupled lipoxin A4 (LXA4) receptor formyl peptide receptor 2 (FPR2)/ALX to resolve inflammation at nanomolar concentrations. There are several lines of evidence indicating possible anti-fibrotic properties of LXA4. It has been studied to inhibit connective tissue growth factor-induced proliferation and to interfere with TGF-p dependent pro-fibrotic properties of lung myofibroblasts. LXA4 also attenuated experimental renal fibrosis and inhibited epithelial to mesenchymal transition of renal epithelial cells in proximal tubules. Presently, it's role in reducing desmoplasia is being explored for anti-cancer application to inhibit cancer progression and metastasis in pancreatic tumors.

LXA4 is rapidly metabolized by human monocytes by dehydrogenation and reduction to 13,14-dihydro LXA4. Therefore, the quest for stable and hydrophilic analogs of LXA4 to resist rapid enzymatic inactivation and to prolong their duration of action is of great relevance. Commercially, lipoxins are available as a solution in EtOH, DMF or DMSO. Incorporating LXA4 into a liposomal bilayer to develop a stable, injectable, sustained-release formulation with room for remote-loading of a chemotherapeutic for targeted therapy in PDAC. Considering the immunogenic properties of irinotecan, we proposed that the development of a dual-delivery liposome comprising a chemotherapeutic agent, and a pro-resolving, anti-fibrotic lipid could present a therapeutic strategy for the following reasons:
(1) LXA4 mediated attenuation of the TGF-0 pathway in the TME will desist activation of tumor resident PSCs, potentially reducing stromogenesis.
(ii) By alleviating the stromal burden, the LXA4-irinotecan liposomes would enhance sensitivity of tumor cells to the ablative effects of irinotecan as well as enhance intratumoral trafficking of immune cells.
(iii) Irinotecan mediated induction of ICD would potentiate training of antigen-specific cytotoxic T cells, thus enabling regression of primary and metastatic lesions.

In this example, we describe the development of a dual drug liposome for the co-delivery of LXA4 that is embedded in the lipid bilayer while irinotecan is remotely imported into the aqueous interior. We present data showing that LXA4 could be successfully incorporated into a liposomal lipid bilayer, which could subsequently be used for remote loading of Irinotecan. The rationale behind this dual-delivery liposomal platform is premised on combining the anti-fibrotic/desmoplastic role of lipoxins and the ablative and immunogenic effects of irinotecan. We demonstrate the functional effectiveness of LXA4 liposomes in vitro by their ability to interfere in TGF-beta activity and inhibit the production of IL6 from human PSCs. In a preliminary subcutaneous KPC model, we also show treatment with LXA4-irinotecan liposomes to slow down tumor progression compared to no treatment controls. The tumors were harvested upon termination of the study to perform histopathological analyses, in which we observed a significant reduction of collagen content in the tumor microenvironment with a concomitant increase in the ratio of cytotoxic to regulatory T lymphocytes.

### Results

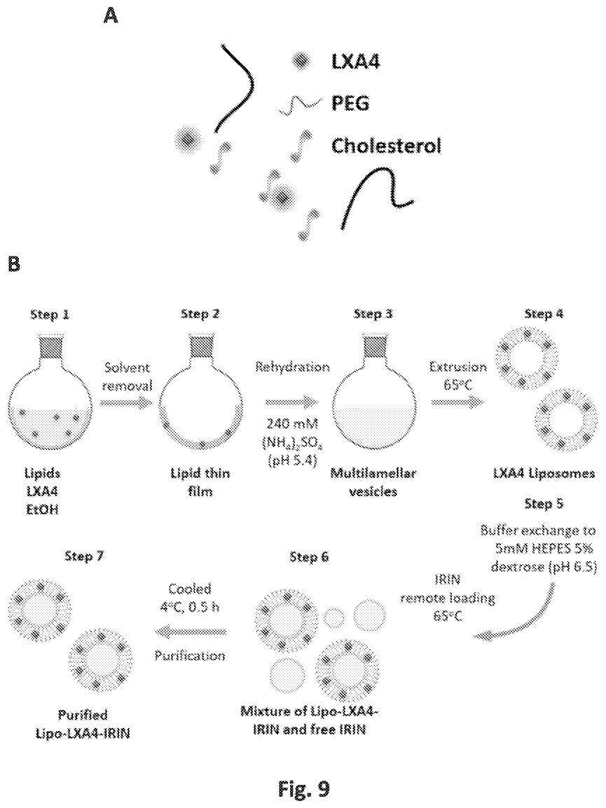

FIG. 9 shows the preparation of LXA4 embedded lipid bilayer for liposome synthesis. Panel A shows a schematic showing incorporation of lipophilic LXA4 into a lipid bilayer structure. Panels B shows a schematic showing synthesis methodology of liposomal LXA4, remotely loaded with irinotecan (IRIN). Briefly, lipids such as DSPC, cholesterol and DSPE-PEG₂ₖ were dissolved in ethanol and mixed with LXA4 in a molar ratio of 55.43:39.6:4.7:0.2, respectively (see Table 7), in a round bottomed glass flask. The solvent was evaporated, leaving an evenly deposited thin lipid film. The dried film was rehydrated in ammonium sulfate solution (pH 5.4) to form a cloudy suspension of multi-lamellar vesicles, followed by extrusion through a gradient of filters to generate liposomes of desired size. For remote drug loading of IRIN, the buffer in which the LXA4-liposomes were suspended was changed to a HEPES-buffered dextrose solution (5 mM HEPES, 5% dextrose, pH 6.5) by Amicon Ultra centrifugal dialysis tubes (30,000 MWCO). The LXA4-liposomes were then mixed with IRIN and incubated at 65° C. After 1 h, the mixture was cooled at 4° C. for 0.5 h. To remove the unloaded IRIN from the buffer, the loaded LXA4-iposomes (Lipo-LXA4-IRIN) was purified by Amicon Ultra centrifugal dialysis tubes (30,000 MWCO).

For use in animal models, a large-scale batch synthesis was performed with a microfluidic device, Benchtop NanoAssembr which allows blending of inner aqueous phase with lipid mixture solvent stream, with active mixing.

TABLE 7
Bilayer composition of liposomes.

| Lipid (Mol %) | DSCP | CHOL | DSPE-PEG_{2 kDa} | LXA4 |
|---|---|---|---|---|
| Lipo-IRIN | 55.8 | 39 | 5 | — |
| Lipo-LXA4-IRINDSPC | 55.43 | 39.6 | 4.7 | 0.2 |

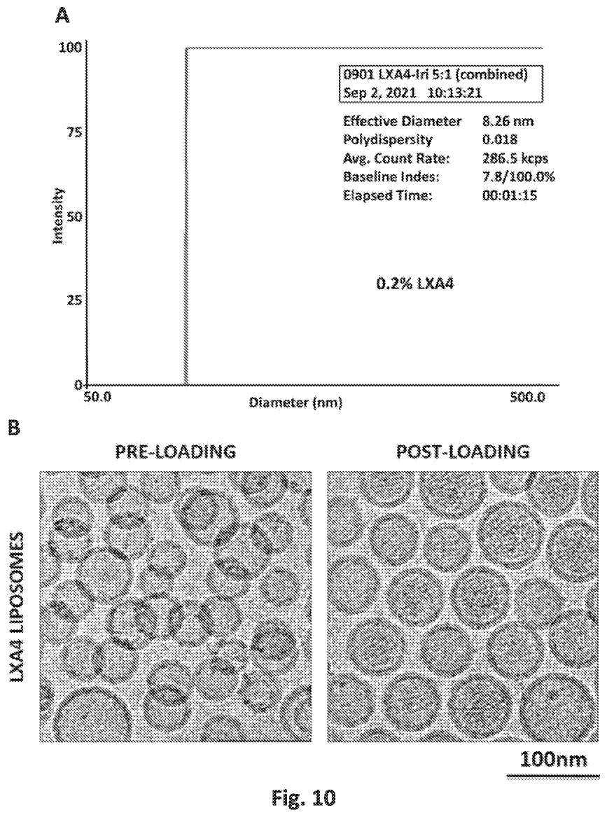

FIG. 10, panels A-B, shows the characterization of the liposomes. As shown in panel A, the liposomes were characterized for size and zeta potential and the drug loading capacity was determined with a ZETAPALS instrument (Brookhaven Instruments Corporation) and UV spectroscopy respectively. We also imaged the resulting liposomes (panel B) with cryo EM to determine the morphology, uniformity and structural integrity of the lipid bilayer coating. The cryo-electron images of the liposomes were obtained with a cryoEM microscope (TF20 FEI TecnaiG2).

The images showed uniform liposomes with a clear unilamellar bilayer structure and large aqueous cores. After remote loading, the presence of drug was visible by a darker contrast of the liposomal interior. The liposomes were ˜80-90 nm in size with a negative zeta potential (see, Table 8).

TABLE 8
Characterization of Lipo-LXA4 and Lipo-LXA4 IRIN
for hydrodynamic size, polydispersity Index (PDI),
zeta potential and drug loading capacities.

| | | | Zeta | IRIN |
|---|---|---|---|---|
| | Size | | potential | loading |
| Sample | (nm) | PDI | (mV) | capacity (%) |
| LXA4 liposome | 88.2 | 0.15 | −3.06 ± 2.1 | — |
| Lipo-LXA4-IRIN | 82.6 | 0.02 | −6.5 ± 0.75 | 25.5 |

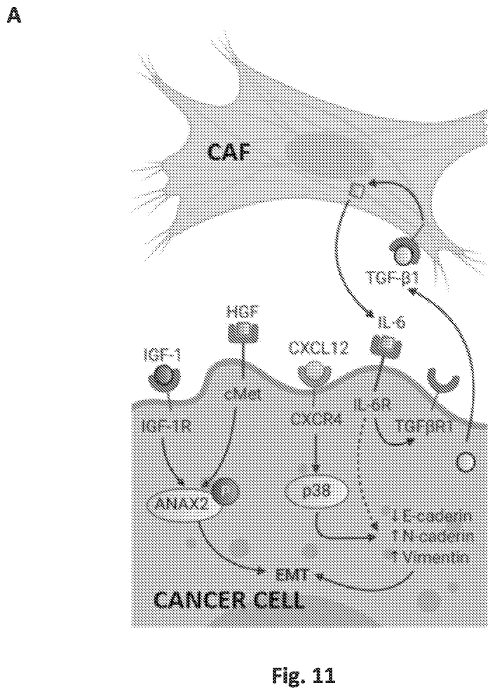

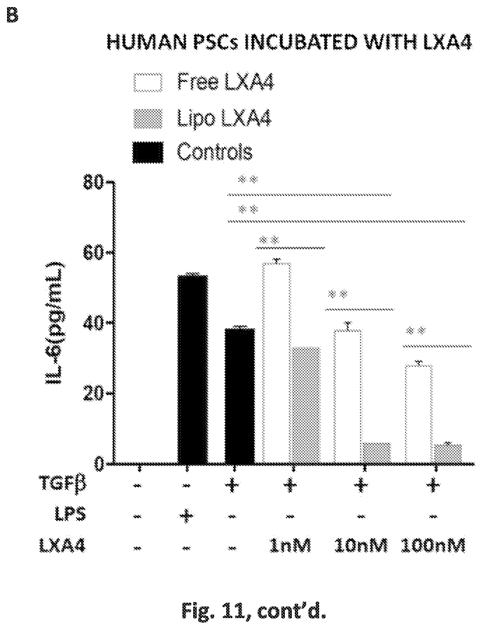

FIG. 11, panels A-B, shows in vitro functional characterization of Lipo-LXA4-IRIN on human pancreatic stellate cells (hPSCs). Panel A shows a schematic showing the TGF-p mediated cross-talk between cancer-associated fibroblasts (CAF) and cancer cells and involvement of cytokines such as IL6 that induces cancer cell growth. Panel B shows LXA4 mediated inhibition of IL6 secretion from TGF-p activated hPSCs detected from culture supernatant by ELISA.

HPaSteCs were seeded at 2500 cells/well in a 96-well plate and activated with TGF-p (20 ng/mL) overnight. The next day, hPSCs were treated with 1-100 nM of LXA4 in free and liposomal form. After 48 h of incubation, the supernatant was collected and analysed for IL-6 secretion with ELISA. Treatment with LXA4 not only significantly inhibited IL-6 release in a concentration-dependent manner, but liposomal LXA4 showed the most robust effects at a concentration of 10 nM compared to positive controls and free LXA4. A possible explanation is provided by the schematic in FIG. 11, panel A.

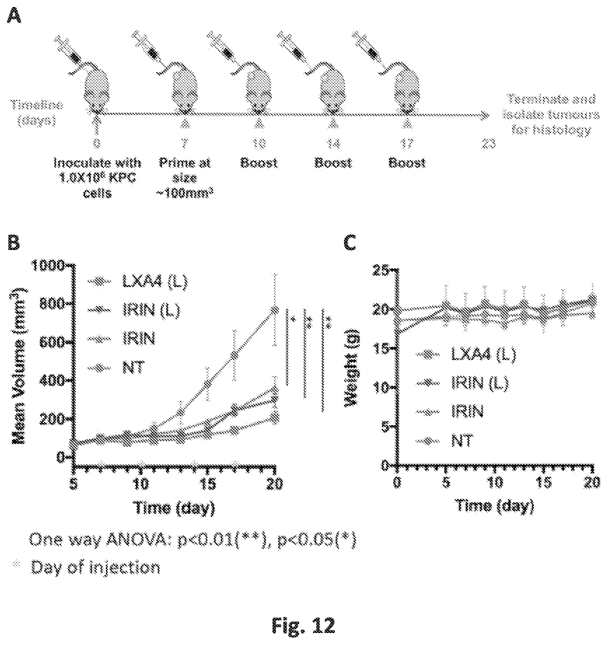

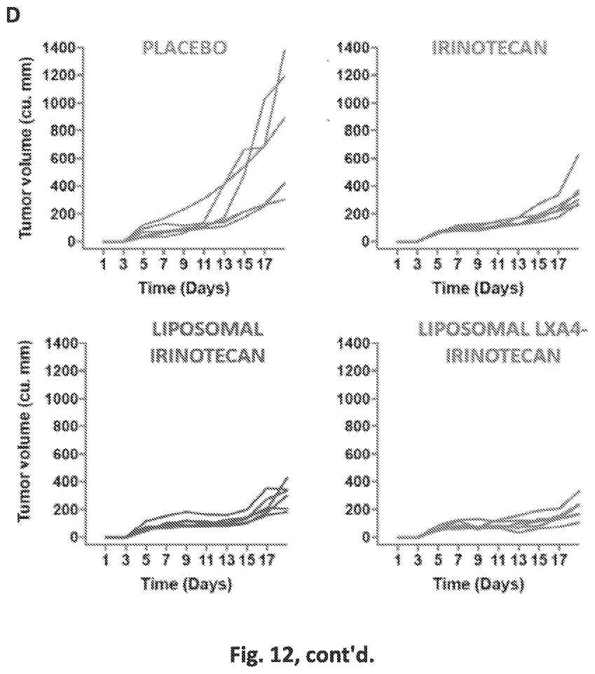

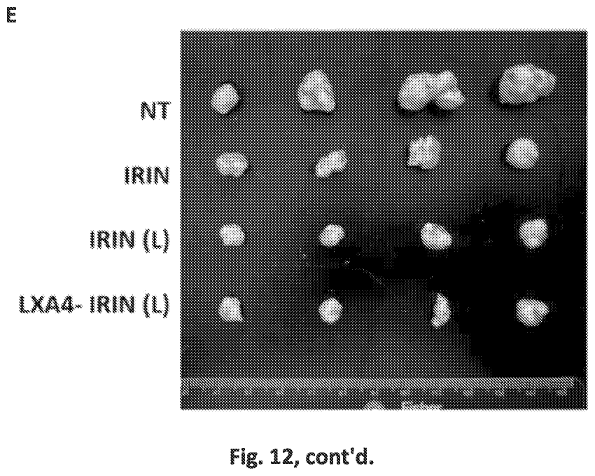

FIG. 12, panels A-E shows in vivo therapeutic efficacy of the dual-delivery lipo-LXA4-IRIN in a subcutaneous KPC pancreatic cancer model. Panel A shows a schematic outlining the implantation and treatment timeline of the subcutaneous KPC model. Briefly, 1×10⁶ KPC cells suspended in PBS/Matrigel (1:1 v/v) solution was injected subcutaneously into the right flank of female B6129SF1/J mice (8-10 weeks old). After 7 days, when tumors were palpable, the mice were randomly divided into 4 groups (n=8) and injected intravenously with the following treatments at an interval of 3-4 days for a total of 4 injections per mouse: (i) saline (no treatment control, NT), (ii) free irinotecan, (iii) liposomal irinotecan (Lipo-IRIN) and (iv) lipo-LXA4-IRIN. Irinotecan was administered at 40 mg/kg while LXA4 was dosed at 1.8 mg/kg. Panel B shows mean tumor volume across the treatment groups over the duration of the study depicting the growth kinetics. Tumor size was measured every 2 days, and tumor volume was calculated by the formula: tumor volume (mm³)=0.52×(length in mm)×(width in mm)². Panel C shows the average body weights of the treated mice across the treatment groups to depict the overall health of the animal over the duration of the study. Panel D shows spaghetti plots showing the growth of individual tumors in each treatment group. Panel E shows images of the tumors that were excised upon termination of the experiment.

Data from the tumor volume curve, demonstrated that lipo-LXA4-IRIN treated mice had the slowest growing tumors compared to the monotherapy controls. However, the difference was only modest and not statistically significant in comparing the two liposomal treatment groups. One interpretation of the data could be the lack of a robust stroma in a subcutaneous KPC model, which limits the extent towards which LXA4 can realistically interfere in tumor growth. To understand the effects of LXA4 delivery and a synergy between the two components we further performed collagen estimation and histopathological analysis. The average weights of mice across treatment groups showed no significant decline, reflecting all treatments to be well tolerated and non-toxic. This observation was further bolstered by a CBC, liver and renal function panel that showed values consistently within normal range and not significantly different between the treatment groups (data not shown).

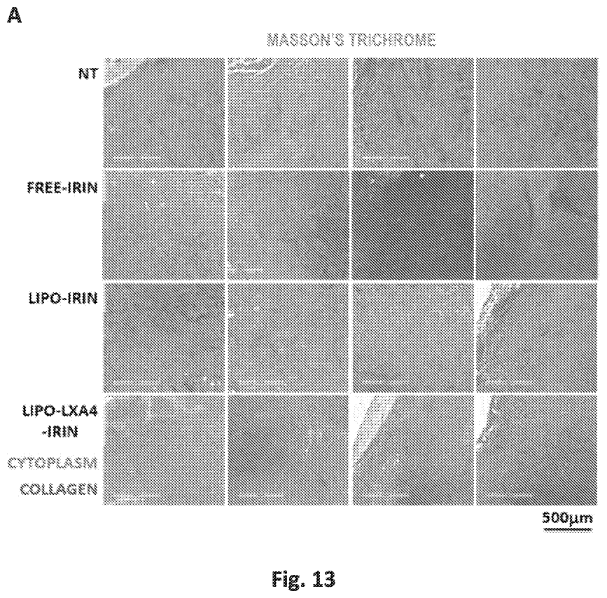

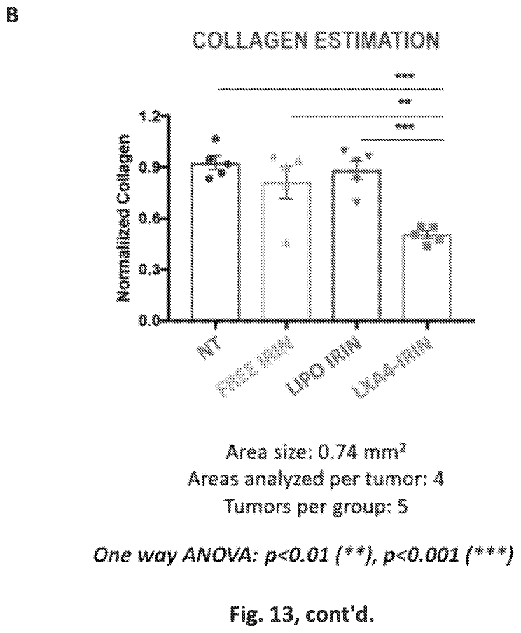

FIG. 13, panels A-B, shows the effect of LXA4 on the severity of desmoplasia estimated by collagen content in the KPC tumor microenvironment. Panel A shows images showing paraffin embedded tumor sections for each treatment group, sectioned at 4 μm stained for Masson's trichrome. Panel B shows quantification of collagen positive areas, normalized over no treatment control.

From the image quantification data, we observed that tumors from mice treated with lipo-LXA4-IRIN showed a statistically significant decrease in mean collagen content, which is a reflection of desmoplasia. This phenotype could be attributed entirely to the efficacy of LXA4 since the difference between lipo-IRIN and lipo-LXA4-IRIN is statistically significant as well. It also demonstrates a potential synergistic effect between pro-resolving lipid and chemotherapy on the stromal composition.

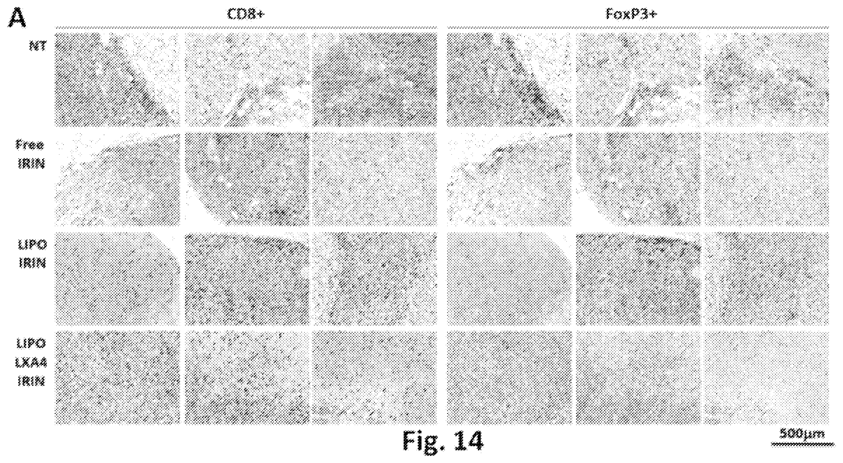

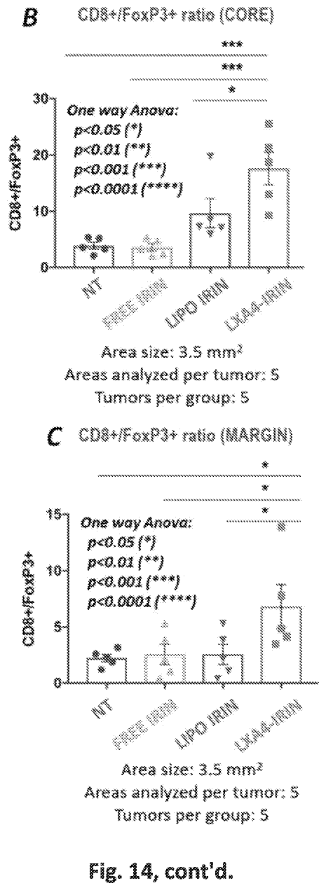

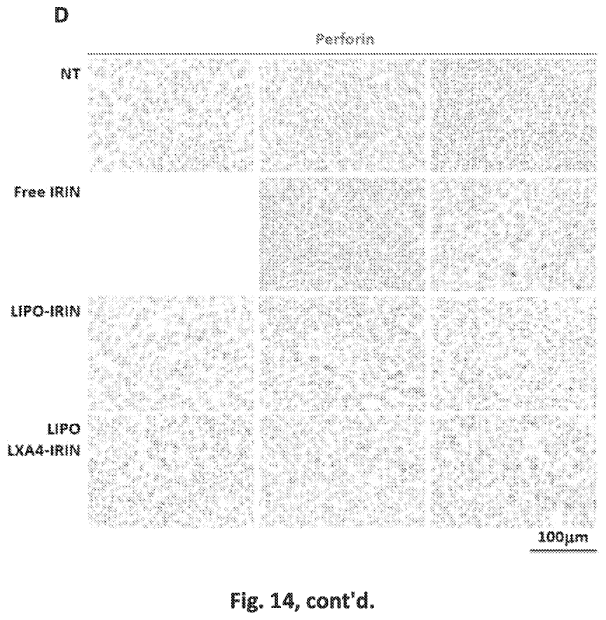

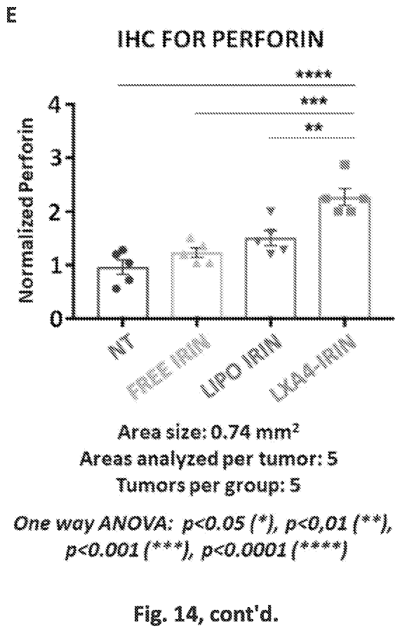

FIG. 14, panels A-E, shows the effect of lipo-LXA4-IRIN on the immune potentiation estimated by immunohistochemistry for T lymphocytes in the KPC tumor microenvironment. Panel A shows representative IHC images of tumor sections stained for Perforin that serves as a marker for cytotoxic activity of CD8+ T cells. Panel B shows quantification of perforin positive signals, normalized over no treatment control.

The immunohistochemical analysis of perforin added an extra dimension of understanding to the change in the immune cell profile for the KPC tumor microenvironment. The lipo-LXA4-IRIN treatment showed significantly higher intensity of the perforin signal compared to all the other treatment group. The free irinotecan and lipo-IRIN treatments also showed an increase in the perforin levels compared to the no-treatment control. This indicates a higher antigen-specific immune response with the dual delivery LXA4-IRIN liposomes.

In order to demonstrate the impact on a tumor model with more rigorous stroma, we are undertaking further studies in an orthotopic KPC model, which is characterized by a more fibrotic stroma that is also known to exert more significant immune suppressive effects than the subcutaneous model.

### Example 3

### Nanocarrier Co-Formulation for Delivery of a TLR7 Agonist Plus an Immunogenic Cell Death Stimulus Triggers Effective Pancreatic Cancer Chemoimmunotherapy

### Summary of Example 3

Although toll-like receptor (TLR) agonists hold great promise as immune modulators for reprogramming the suppressive immune landscape in pancreatic ductal adenocarcinoma (PDAC), their use is limited by poor pharmacokinetics (PK) and off-target systemic inflammatory effects. To overcome these challenges as well as to attain drug synergy, we developed a lipid bilayer (LB)-coated mesoporous silica nanoparticle (silicasome) platform for co-delivery of the TLR7/8 agonist, 3M-052, with the immunogenic chemotherapeutic agent, irinotecan. This was accomplished by incorporating the C18 lipid tail of 3M-052 in the coated LB, which is also used for irinotecan remote loading into the porous interior. Not only did the co-formulated carrier improve PK but strengthened the irinotecan-induced immunogenic cell death (ICD) response by 3M-052-mediated dendritic cell activation at the tumor site, as well as participating lymph nodes. The accompanying increase in CD8⁺ T-cell infiltration along with a reduced number of regulatory T-cells was associated with tumor shrinkage and metastasis disappearance in subcutaneous and orthotopic KRAS-mediated pancreatic carcinoma (KPC) tumor models. Moreover, this therapeutic outcome was accomplished without drug or nanocarrier toxicity. All considered, dual-delivery strategies that combine chemo-immunotherapy with co-formulated TLR agonists or other lipid-soluble immune modulators holds great promise for intervening in heterogeneous PDAC immune landscapes.

### Introduction to Example 3

Pancreatic ductal adenocarcinoma (PDAC) is the third leading cause of cancer death in the United States, with a five-year survival rate of ˜11%.^{[1,2]} The poor prognosis is due to late clinical presentation, as well as interference in drug delivery and drug resistance by the abundant dysplastic stroma.^{[3]} To improve the pharmacokinetics (PK) of drug delivery, a number of nanocarriers have been introduced for PDAC treatment more recently, including the irinotecan-delivering liposome, Onivyde, and an albumin-paclitaxel nanocarrier, nab-paclitaxel (Abraxane).^{[4,5]} While the principal consideration for nab-paclitaxel development was to overcome the toxicity of an incipient, Onivyde was developed to improve the efficacy of combination chemotherapy in patients with metastatic disease and who failed to respond to gemcitabine treatment. More recently, the pegylated irinotecan liposome was also approved as first-line therapy for untreated, locally advanced, metastatic disease in combination with oxaliplatin, 5-fluorouracil, and leucovorin.

However, while Onivyde improved therapeutic efficacy, the drug received a black box warning for severe diarrhea and neutropenia, most likely related to carrier leakage resulting from the impact of plasma proteins, complement and circulatory shear forces on the intactness of the lipid bilayer (LB).^{[4]} These shortcomings were instrumental in the development of the silicasomes, which make use of a supported LB for coating of mesoporous silica nanoparticles (MSNPs).^{[6-7]} Not only is the supported LB more stable, but also allows irinotecan remote loading and high drug import into the porous interior. This allowed us to accomplish improved irinotecan delivery to orthotopic KRAS-dependent pancreatic cancer (KPC) tumors compared to Onivyde, in addition to demonstrating the absence of bone marrow toxicity, blunting of intestinal villi, and hepatotoxicity, which are seen during treatment with free or liposomal irinotecan.^{[8]}

An exciting recent advance for silicasome use and irinotecan delivery is the demonstration that this chemo agent also induced immunogenic cell death (ICD), in addition to its role as a topoisomerase I inhibitor.^{[9]} The robust induction of ICD was explained by a collateral irinotecan effect on lysosomal alkalization, leading to interference in autophagy flux and generation of endoplasmic reticulum stress.^{[10,11]} This culminates in a robust generation of immune danger signals to assist the specialized cell death response, which promotes dying cancer cell uptake by bystander antigen-presenting cells (APCs). This action involves calreticulin (CRT) expression on the dying tumor cell surface, further assisted by high mobility group protein B1 (HMGB1) and ATP release, which function as adjuvant stimuli to promote dendritic cell (DC) activation and maturation.^{[12,13]} The ICD process can be likened to an endogenous tumor vaccination response, allowing tumor antigen presentation to naïve T-cells by DC in secondary lymphoid organs (lymph nodes and spleen). The activated CD8+ cytotoxic T-cells (CTLs) return to the primary tumor site to complete the cancer immunity cycle.^{[14]}

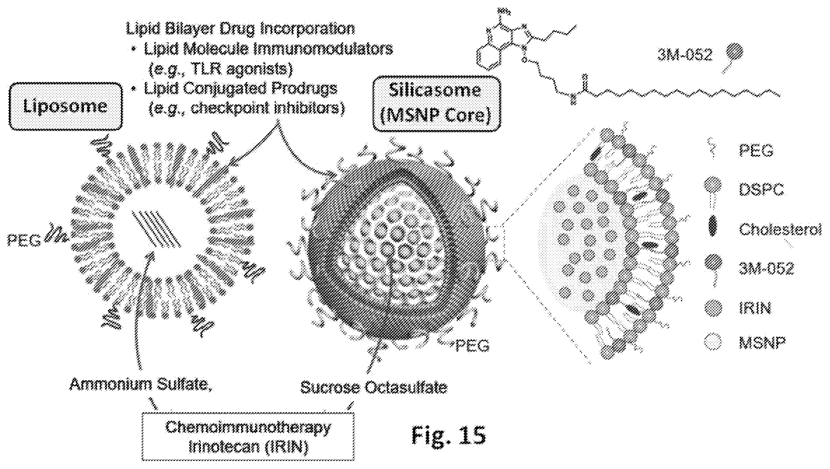

Not only did irinotecan induce an ICD response, but could also be seen to promote PD-L1 expression as a consequence of endoplasmic reticulum stress.^{[9]} This allowed us to strengthen the ICD response by co-administration of a monoclonal antibody that blocks PD-L1 binding to PD-1. The combinatorial effect raised the possibility of using the multifunctional features of the silicasome to co-formulate immunomodulators, selected to intervene in a host of immune challenges in the PDAC immune landscape such as a paucity of neoantigens, poor recruitment of APC, checkpoint receptor-mediated immune escape and immune suppressive cellular components in the tumor microenvironment (TME).^{[15]} A facile design approach emerging from this thinking was to use the packaging space for incorporation of ICD-inducing chemo agents (such as irinotecan) into the particle pores, while including synthetic lipid immunomodulators such as toll-like receptor (TLR) agonists or lipid-conjugated prodrugs into the LB to deliver adjuvant stimuli or interfere in immune checkpoint pathways (FIG. 15).

The ubiquitously expressed toll-like receptor (TLR) family functions as pattern recognition receptors (PRRs), capable of activating the innate immune system in response to pathogen-associated molecular patterns (PAMPs) or damage-associated molecular patterns (DAMPs).^{[16,17]} All TLRs share a typical structural motif in the form of leucine-rich repeats, which upon dimerization by cognate ligands, induce recruitment of adapter and signaling molecules providing transcriptional activation in innate immune cells.^{[18-20]} For instance, TLR7, an endosomal-expressed receptor, is capable of sensing viral single-stranded (ss) RNA, leading to NF-κB/AP1 and IRF-mediated expression of type I IFNs, pro-inflammatory cytokines, and co-stimulatory molecules (e.g., CD80 and CD86) in antigen presenting cells (APC), including dendritic cells.^{[21]} This introduces TLR7 and a closely related endosomal homolog, TLR8, as attractive therapeutically assessable immune modulators for boosting innate immunity in the context of infectious disease and tumor immune landscapes. Historically, the first patent filing was for a drug known as imiquimod (a.k.a. R837) to treat genital warts and basal cell carcinomas, followed by introduction of resiquimod (R848), which is 10-fold to 100-fold times more potent than R837.^{[22,23]} However, while these agents triggered robust immune activation during conducting of clinical trials, a major setback was the occurrence of systemic on-target but off-tumor inflammatory reactions.^{[20,24]} Consequently, drug advancement focused on local or encapsulated drug delivery. For instance, 3M-052, an imidazoquinoline compound linked to a C18 lipid tail, was used to construct liposomal, polylactic co-glycolic acid (PLGA) and small lipid nanoparticles, to provide adjuvant stimuli for attempts at infectious disease vaccination.^{[25-28]} In addition, the small molecule TLR7 agonist, 1V209, was used for cholesterol conjugation (1V209-Cho) and incorporation into a nanocarrier capable of reaching lymph nodes and boosting immunotherapy responses in CT26 colorectal, 4T1 breast cancer, and Pan02 PDAC tumor models.^{[29]}. This included demonstration that that improved DC activation leads to the boosting of immune responses without systemic side effects.

Based on this background as well as the awareness of poor dendritic cell function in human and murine PDAC microenvironments, we hypothesized that the 3M-052 lipid tail would be ideal for LB incorporation of irinotecan-delivering silicasomes, with the possibility of strengthening the chemoimmunotherapy response by DC activation. To test this hypothesis, we developed a method to incorporate 3M-052 into a LB before applying it to the coating of MSNP. The carrier was then used for irinotecan remote loading before intravenous (IV) injection in PDAC tumor-bearing mice. We demonstrate generation of a synergistic anti-PDAC immune response by improved drug delivery, including through boosting of APC activity that also involve regional lymph nodes.

### Results

### Development of Dual-Drug Silicasomes that Deliver Irinotecan Plus 3M-052

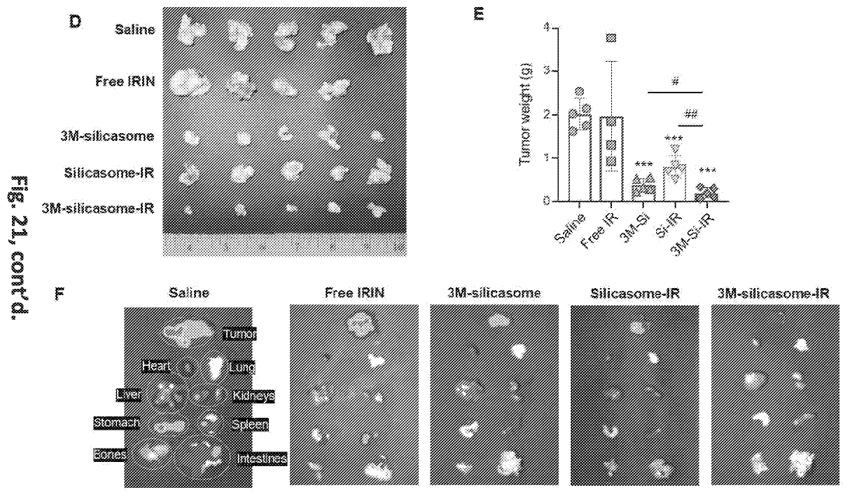

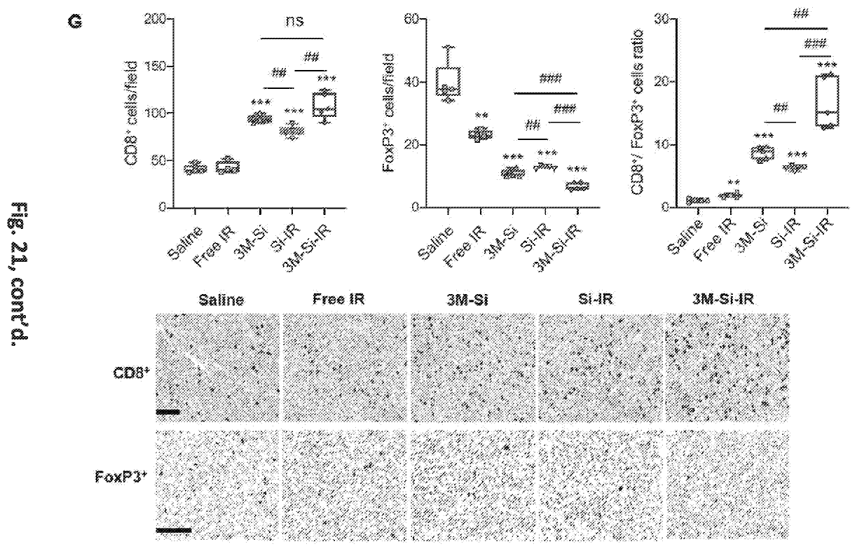

The envisaged design principle for accomplishing a co-formulated silicasome carrier requires remote loading of irinotecan (IRIN) into the porous packaging space of the silicasome using a protonating agent, while incorporating 3M-052 (a.k.a. Telratolimod) into the lipid bilayer (LB) (FIG. 15). To develop an appropriate bilayer composition, we experimented with a series of lipid biofilms to determine whether 3M-052 can be stably incorporated into a LB. The optimal lipid bilayer composition was achieved with DSPC/Chol/DSPE-PEG2000/3M-052 in the molar ratio of 55.5:38.5:2.7:3.3); this yielded liposomes with average size of 136 nm and 3M-052 loading capacity of 1.5% (FIG. 22). We also demonstrated achievement of an IRIN remote loading capacity of 18.6% through the use of (NH₄)₂SO₄ as trapping agent. Additional physicochemical characteristics of this carrier, designated 3M-liposome-IRIN, appear in FIG. 22.

Successful establishment of a 3M-052 lipid bilayer allowed us to proceed with silicasome construction. Controlled synthesis of spherical MSNPs was accomplished using a heated CTAC solution for TEOS addition, as previously described by us.^{[6-8]} The particles were soaked in a trapping agent, TEA₈SOS, before sonicating in a lipid solution of the same composition as described above (FIG. 3, panel A). Following removal of free TEA₈SOS, IRIN remote loading and silicasome purification was accomplished as previously described by us.^{[8]} This yielded a carrier, designated as 3M-silicasome-IR (also abbreviated as 3M-Si-IR). As a control, we also prepared a nanocarrier loaded with 3M-052 only, designated a 3M-silicasome or 3M-Si. Cryo-electron microscopy (CyroEM) of both carriers confirmed the presence of MSNPs with a worm-like porous structure, coated with an intact LB (FIG. 3, panels B and C). Hydrodynamic size measurement showed sizes of ˜112 and ˜127 nm, respectively, for the 3M-silicasome and 3M-silicasome-IR carriers (Table 9). Both carriers had a 3M-025 loading capacity of 2.1%, with the dual delivery carrier capable of achieving an IRIN loading capacity of 38%.

TABLE 9
Physicochemical properties of both silicasomes, including
hydrodynamic size, polydispersity index (PDI), zeta
potential, and drug loading capacities.

| | | | | 3M-052 | IRIN |
|---|---|---|---|---|---|
| | | | Zeta | Loading | loading |
| | Size | | potential | Capacity | capacity |
| Sample | (nm) | PDI | (mV) | (%) | (%) |
| 3M-silicasome | 122.2 ± 1.0 | 0.102 | −9.1 ± 0.9 | 2.1 | — |
| 3M-silicasome- | 127.0 ± 2.9 | 0.066 | −12.8 ± 2.0 | 2.1 | 37.9 |
| IR | | | | | |

### Demonstration of TLR7/8 Activation by Encapsulated 3M-052 in Cellular Studies

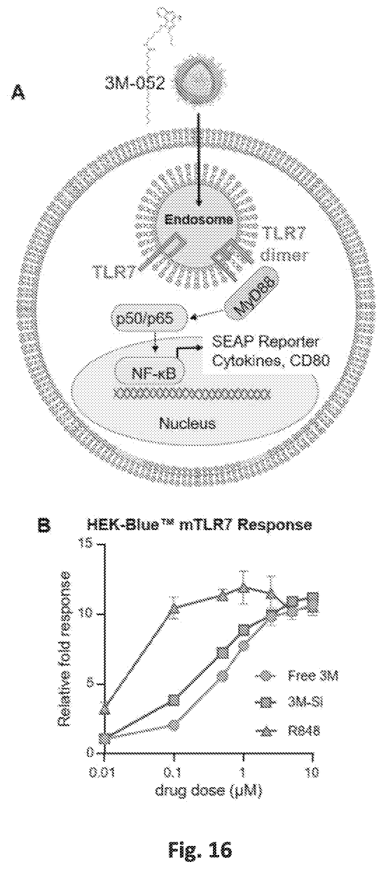

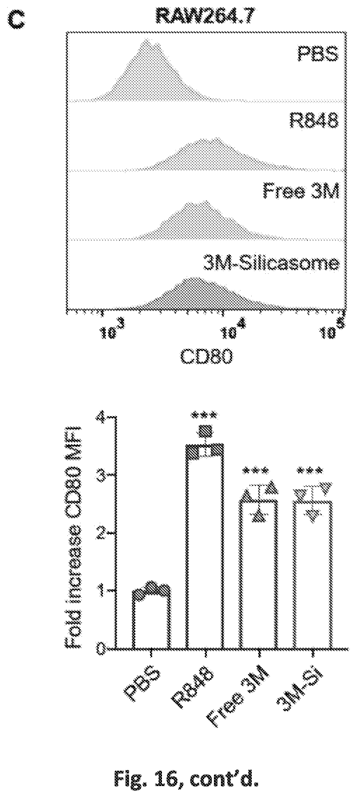

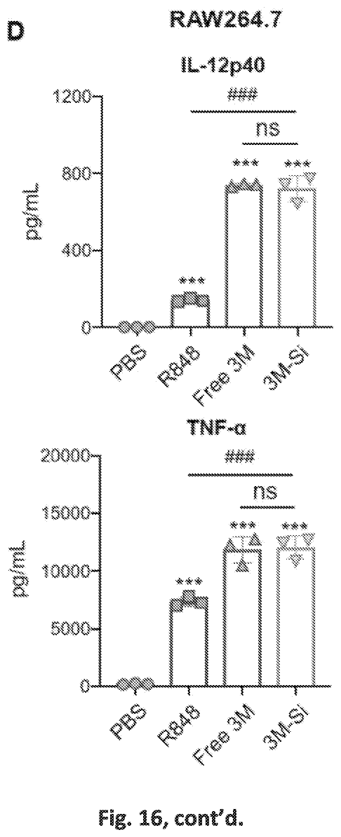

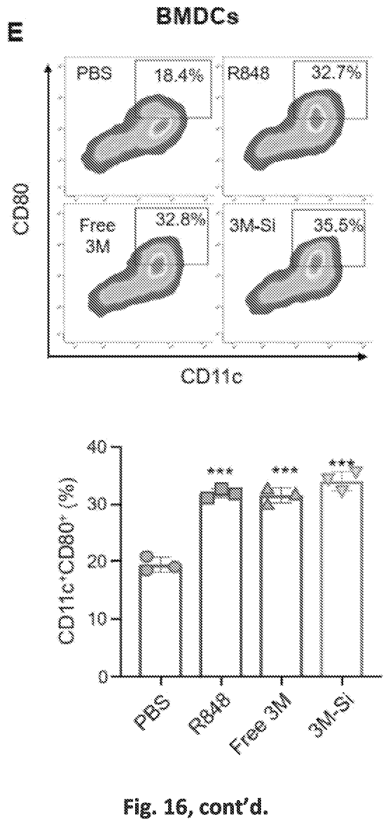

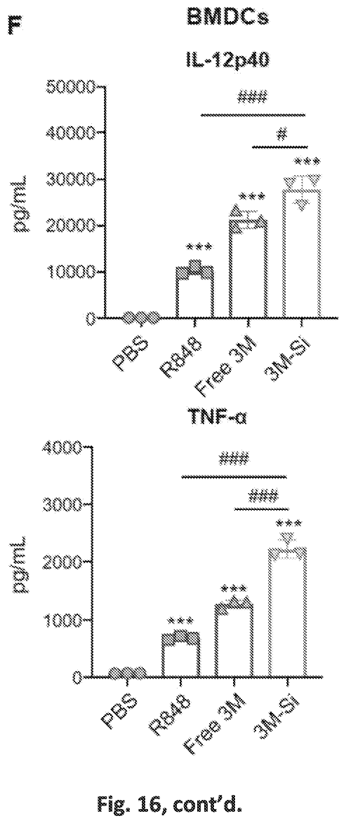

Both the TLR7 and TLR8 receptors are widely expressed in innate immune cells, including DC and macrophages. Their endosomal localization allows intracellular sensing of single-stranded viral RNA, leading to receptor dimerization and triggering of transcriptional activation pathways involved in DC and macrophage activation and maturation (FIG. 16, panel A). More specifically, TLR7 and TLR8 dimerization leads to the recruitment of the adaptor protein, myeloid differentiation primary response 88 (MyD88), which triggers NF-κB/AP-1 mediated signaling cascades that induce production of pro-inflammatory cytokines such as IL-6, IL-12p40, and TNF-α.

To confirm intracellular delivery and attainment of a TLR7 agonist effect for the 3M-silicasome, we used a commercially available HEK-Blue™ cell line, which expresses the murine TLR7 gene and an NF-κB/AP-1-responsive SEAP (secreted embryonic alkaline phosphatase) reporter gene (FIG. 16, panel A). This allows detection of SEAP levels in the culture medium of the cells exposed to 0.01 to 10 μM free R848 (positive control), free 3M-052, or the 3M-silicasome for 20 h (FIG. 16, panel B). Quantification of SEAP levels, using the HEK-Blue™ Detection kit, demonstrated similar dose-response relationships for free and encapsulated 3M-052, with R848.

Having confirmed effective intracellular delivery of TLR7 activation, the next task was to determine the effect of the 3M-silicasome in the ex vivo activation of macrophages and DCs. These assays were performed in RAW264.7 macrophages and murine bone marrow-derived dendritic cells (BMDCs). RAW264.7 and BMDCs were treated with 10 μM each of R848 (positive control), free 3M-052, or the 3M-silicasome for 21 hours before assessing CD80 expression (FIG. 16, panels C and E). The flow cytometry assessment demonstrated a significant increase in cell surface expression of a maturation marker, regulated by NF-κB and AP-1 response elements. We also quantified the release of two key pro-inflammatory cytokines, IL-12p40 and TNF-α by ELISA in the same cell types, using similar stimuli. This confirmed significant cytokine release into the culture supernatants, demonstrating higher responses to free and encapsulated 3M-052 band for R848 ((FIG. 16, panels D and F).

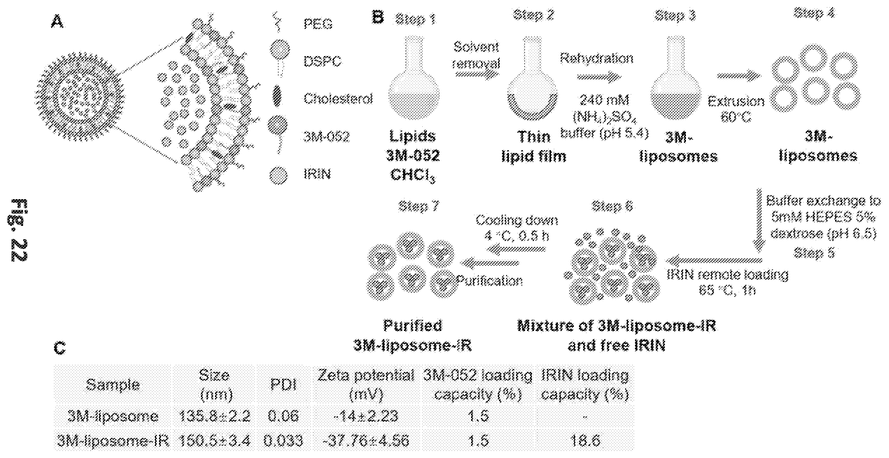

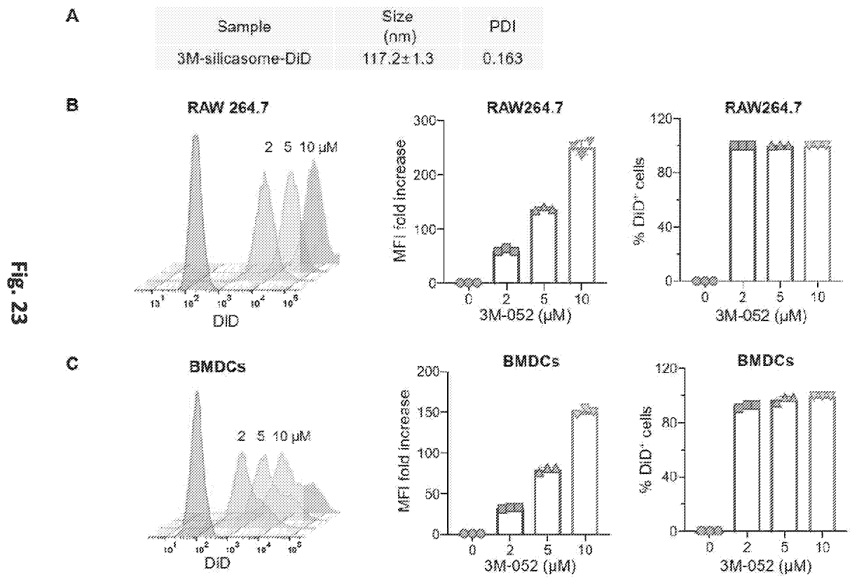

To confirm cellular uptake of the 3M-silicasome in RAW264.7 and BMDC, a cellular study was performed, using a DiD-labeled 3M-silicasome carrier with the characteristics shown in FIG. 23, panel A.

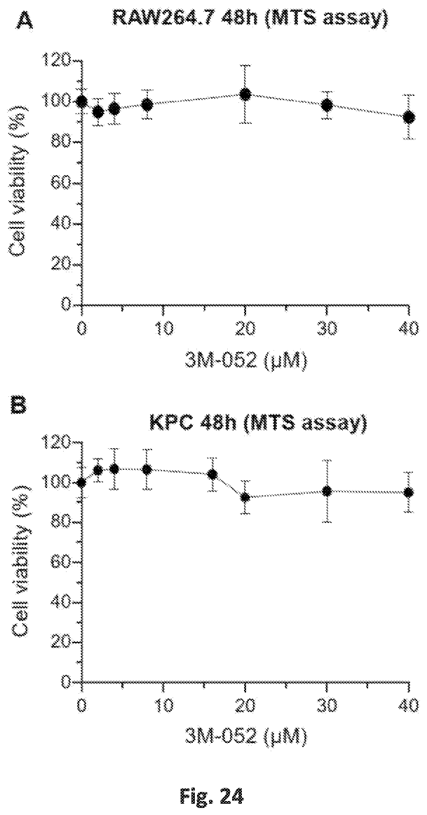

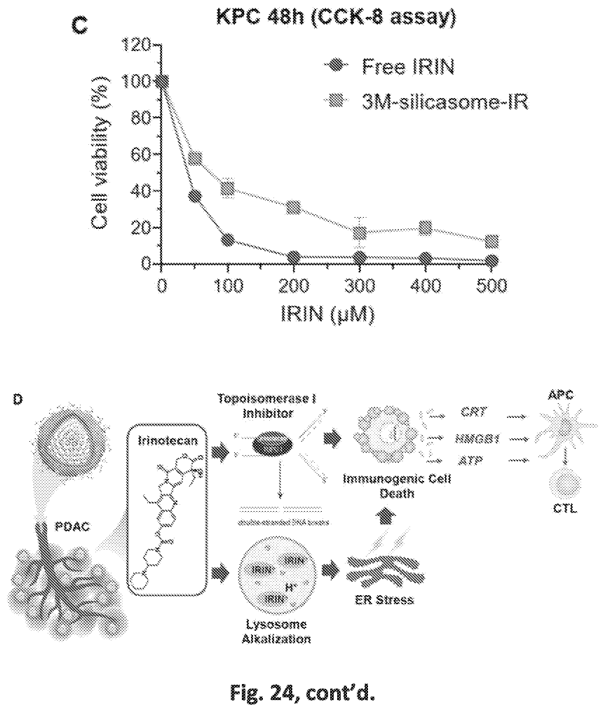

Flow cytometry was used to assess cellular fluorescence following incubation with a carrier dose range delivering incremental amounts of encapsulated 3M-052 (FIG. 23, panels B and C). This demonstrated a dose-related increase in cellular fluorescence with almost all cells engaging in particle association. There was no evidence of toxicity in response to encapsulated 3M-052 in RAW267.4 cells, as well as a pancreatic cancer cell line (KPC) derived from the genetically engineered KRAS mouse pancreas cancer model (Pdx1-cre/LSL-Kras G12D/p53R172H) (FIG. 24, panels A and B). To assess the impact of IRIN delivery on KPC cells, they were exposed to a wide dose range of free and encapsulated IRIN for 48 h (FIG. 24, panel C). This demonstrated dose-dependent cytotoxicity, with a steeper decline in viability in response to the free drug (FIG. 24, panel C). Noteworthy, we have previously demonstrated that the KPC cell death response to IRIN involves a combination of nuclear damage (topoisomerase 1 inhibition) plus triggering of an endoplasmic reticulum (ER) stress response (FIG. 24, panel D).^{[9]} Collectively, this culminates in an ICD response, which is characterized by calreticulin (CRT) expression on the cell surface, as well as HMGB1 and ATP release from the dying cells (FIG. 24, panel D). CRT acts as a “eat-me” signal for APC processing while HMGB1 and ATP act as adjuvants for APC activation and maturation.^{[12,13]}. In vivo studies will further demonstrate the relevance of the mode of cell death to immune response boosting by 3M-052.

### Demonstration of Carrier Therapeutic Efficacy and Immune Activation in a Subcutaneous KPC Tumor Model

KPC cells were subcutaneously injected into the right flank of female B6129SF1/J mice for in vivo experimentation. Following tumor growth to ˜100 mm³, groups of 6-7 animals received IV injection of saline, free 3M-052 (2 mg/kg), free IRIN (40 mg/kg), 3M-silicasome (3M-052, 2 mg/kg) and 3M-silicasome-IR (3M-052, 2 mg/kg; IRIN, 40 mg/kg) every 3 or 4 days on four occasions (FIG. 17, panel A). Subcutaneous tumor sizes were monitored every 2 days. While all treatment modalities significantly impacted the tumor volumes at the harvesting stage compared to saline (p<0.001), the effects of free 3M-052 and free IRIN were significantly reduced compared to treatment with single-drug or dual-drug silicasomes (p<0.01) (FIG. 17, panel B). Moreover, tumor volume reduction in mice treated with the dual-delivery carrier was significantly less (p<0.05) than for the 3M-silicasome. These findings were further corroborated by capture of photographic images displaying tumor size at the time of harvest (FIG. 17, panel C), as well as displaying the growth kinetics of individual tumors, displayed as spaghetti plots (FIG. 17, panel D). Differences in the response outcome between free 3M-052 and the 3M-silicasome suggested that encapsulated delivery is more efficacious in promoting the anti-PDAC immune response at the primary tumor site.

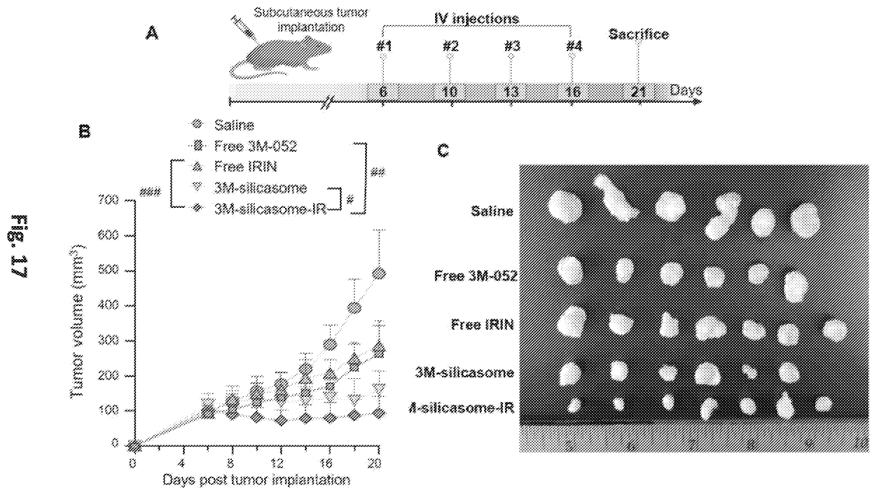

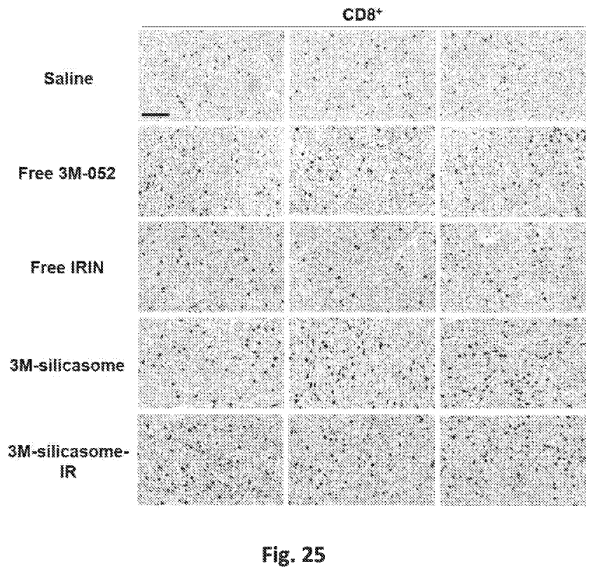

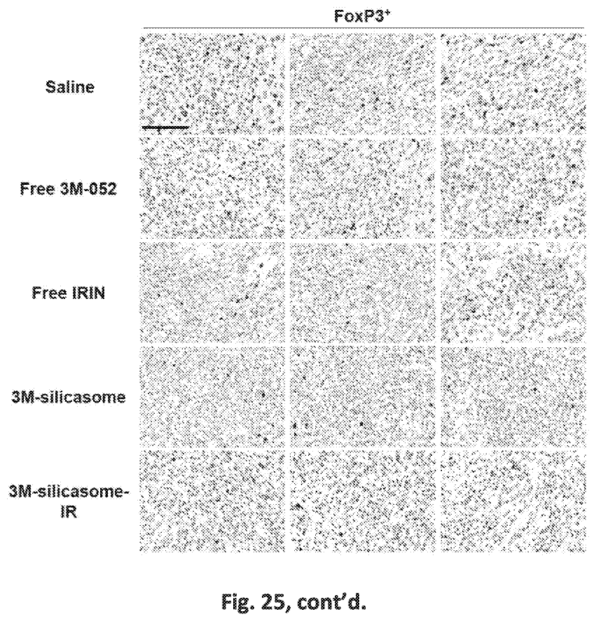

In order to investigate specifics of the immune response induced at the primary cancer site, tumors were harvested from sacrificed animals on day 21 for performance of immunohistochemistry (IHC) analysis. First, we looked at the appearance of CD8+ T-cells, demonstrating that both the 3M-silicasome-IR as well as the 3M-silicasomes could induce significant CD8⁺ CTL recruitment compared to free IRIN or free 3M-052 (FIG. 18, panel A). Representative IHC images are shown underneath the graphic, with the comprehensive data panel appearing in FIG. 25. We also performed IHC staining to assess the presence of FoxP3+ expression on regulatory T-cells (Tregs), which make an important contribution to immune suppressive PDAC landscape.^{[30-32]} Quantification of FoxP3⁺ cell number demonstrated a highly significant reduction of this marker for all treatment modalities, the most robust contribution being that of the 3M-silicasome-IR (FIG. 18, panel A and FIG. 25). Additional display of the number of CD8⁺ vs. FoxP3⁺ T-cells, reflected as a ratio, confirmed that the biggest ratio increase occurred in the dual-delivery carrier compared to other treatment modalities. The magnitude of change is in favor of a synergistic response generation by co-delivery of irinotecan with the TLR7 agonist.

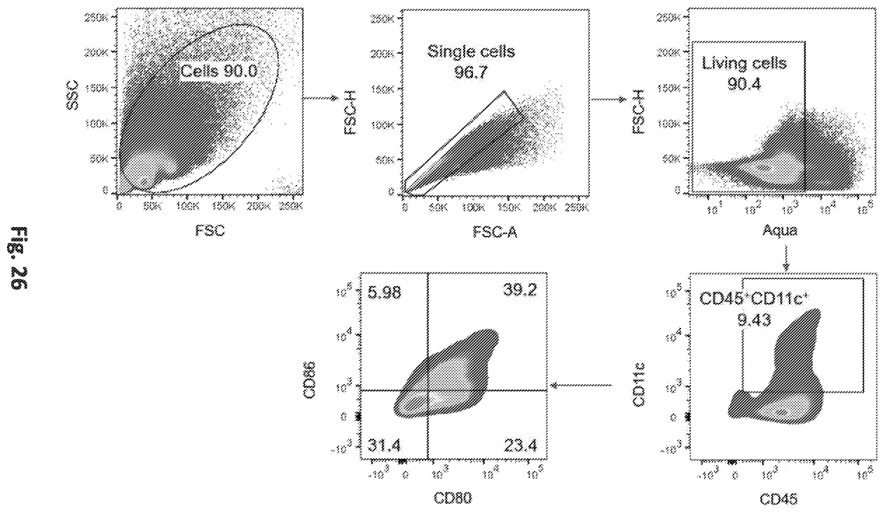

Subcutaneous tumor implantation in the rear animal flank allows lymphatic drainage to the regional inguinal lymph nodes (LNs), which were harvested to assess therapeutic impact on LN-resident dendritic cells (LN-DCs). This reasoning is premised on the possibility that LN participation in the cancer immunity cycle may be able to strengthen the ICD response by increasing recruitment of newly-activated cytotoxic T-cells back to the principal tumor site. Following inguinal LN harvesting and cellular release, initial flow cytometry gating selected singlet live cells (FIG. 26). Further gating on the CD11c⁺/CD45⁺ DC subset was used to quantify CD80 and CD86 expression, representative of the surface receptors on an activated DC subset, participating in T-cell activation. Flow analysis demonstrated an increase in the percentage of CD45⁺/CD11c⁺ LN-DCs in response to free or encapsulated 3M-052 delivery (single and dual-drug carriers), compared to saline and free IRIN (FIG. 18, panel B). Moreover, while CD80 and CD86 expression were up-regulated in response to 4 rounds of treatment with free or encapsulated 3M-052, the most significant increase was obtained for the 3M-052/IRIN silicasome (FIG. 18, panel C). All considered, the dual-drug silicasome was more effective than the free drug or single-drug silicasome for generating anti-PDAC immunity, favoring the notion that TLR7/8 activation is capable of boosting the IRIN-induced ICD response. We also obtained evidence that the response boosting involves LNDC participation as a key component of the cancer immunity cycle.

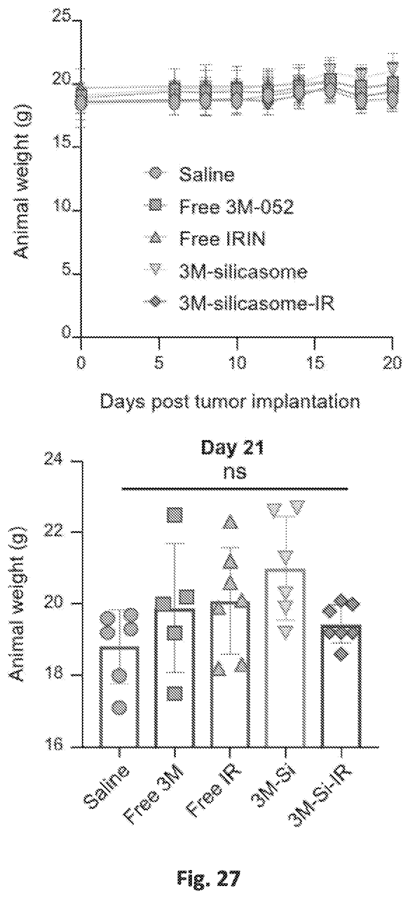

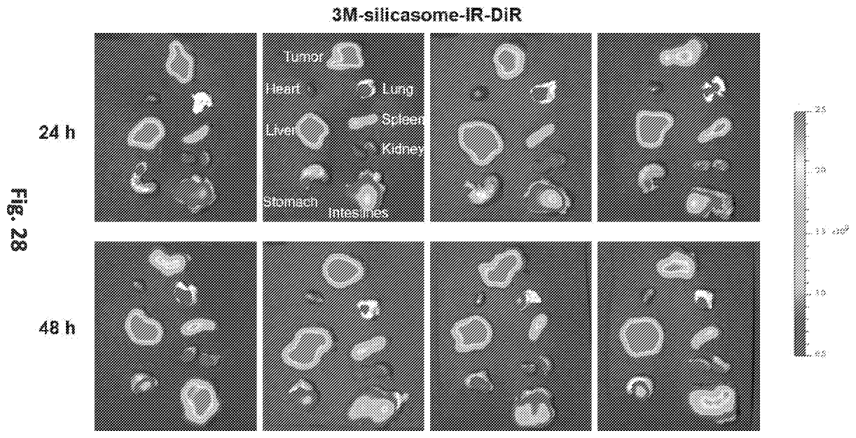

Noteworthy, these therapeutic outcomes were achieved without a significant change in animal body weight in any of the groups, which is in favor of the absence of systemic toxicity (FIG. 27). This notion was further confirmed by blood collection for white blood cell counting and assessment of kidney function (creatinine and urea levels), liver enzymes (ALP, AST, and ALT), blood calcium and phosphate levels, as demonstrated in Table 10. These results are also congruent with previous demonstration of improved safety of our IRIN nanocarrier, in which a supported LB is superior to a liposomal LB.^{[1]} Table 10. A subcutaneous KPC tumor model was established in B6129SF1/J mice. Tumor-bearing mice were treated with saline, free 3M-052, free IRIN, 3M-silicasome, and 3M-silicasome-IR at dose equivalents of 2 mg/kg and 40 mg/kg for 3M-052 and IRIN, respectively, every 3-4 days, for a total of 4 IV injections. Blood was collected from the sacrificed animals on day 21. The biochemical parameters were assayed by UCLA Division of Laboratory Animal Medicine (DLAM) diagnostic laboratory services. These parameters include white blood cell (WBC), alkaline phosphatase (ALP), aspartate aminotransferase (AST), alanine aminotransferase (ALT), blood urea nitrogen (BUN), creatinine, calcium, and phosphorus. There were no statistically significant differences between the saline control group and other treatment modalities.

| | WBC | ALP | AST | ALT |
|---|---|---|---|---|
| Treatments | (10 \hat{ }3/μL) | (U/L) | (U/L) | (U/L) |
| Saline | 5.1 ± 2.0 | 94.7 ± 15.6 | 56.7 ± 3.5 | 18.3 ± 3.8 |
| Free 3M | 3.5 ± 0.1 | 64.3 ± 7.6 | 95.0 ± 14.0 | 32.3 ± 11.9 |
| Free IRIN | 3.9 ± 0.5 | 91.0 ± 1.7 | 66.3 ± 22.2 | 20.3 ± 1.5 |
| 3M-Si | 3.7 ± 0.7 | 77.7 ± 8.1 | 61.7 ± 11.9 | 22.3 ± 4.2 |
| 3M-Si-IR | 5.0 ± 1.2 | 80.7 ± 18.5 | 63.3 ± 5.8 | 24.7 ± 4.5 |
| | BUN | Creatinine | Calcium | Phosphorous |
| Treatments | (mg/dL) | (mg/dL) | (mg/dL) | (mg/dL) |
| Saline | 17.7 ± 0.6 | 0.0 ± 0.0 | 8.8 ± 0.3 | 6.7 ± 1.0 |
| Free 3M | 16.0 ± 2.0 | 0.0 ± 0.0 | 8.7 ± 0.2 | 7.2 ± 1.3 |
| Free IRIN | 17.0 ± 1.0 | 0.0 ± 0.1 | 8.9 ± 0.1 | 7.4 ± 0.5 |
| 3M-Si | 14.7 ± 2.1 | 0.1 ± 0.0 | 8.8 ± 0.2 | 6.9 ± 0.6 |
| 3M-Si-IR | 16.3 ± 2.5 | 0.0 ± 0.1 | 8.4 ± 0.2 | 6.8 ± 0.4 |

### Improved Pharmacokinetics of Drug Delivery and Effective Silicasome Biodistribution to Orthotopic Tumors

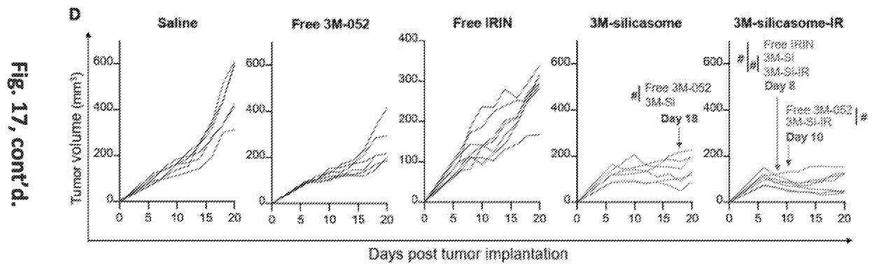

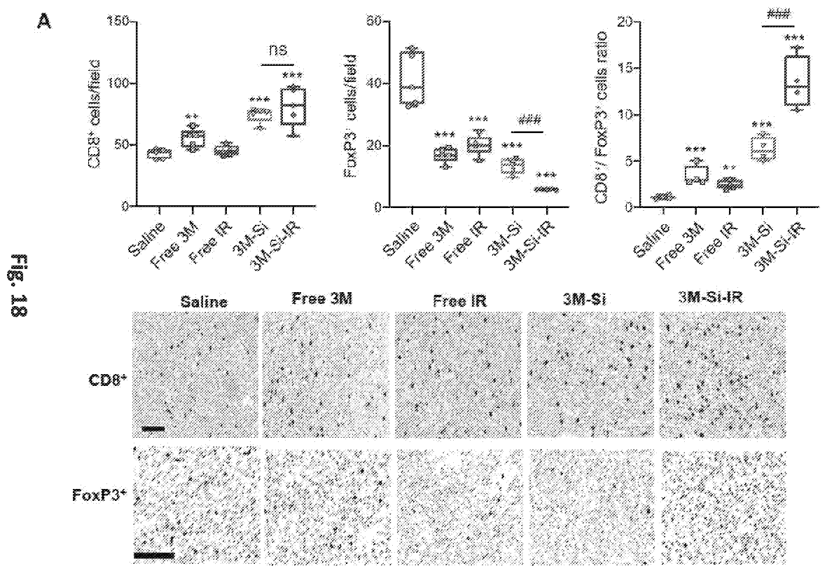

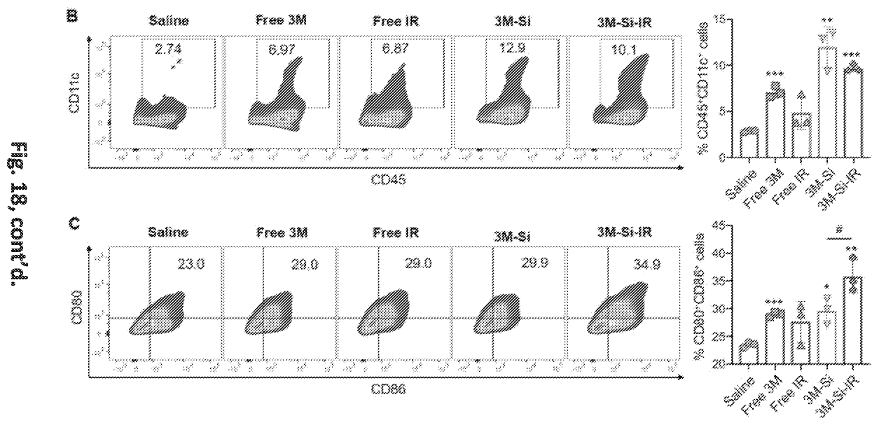

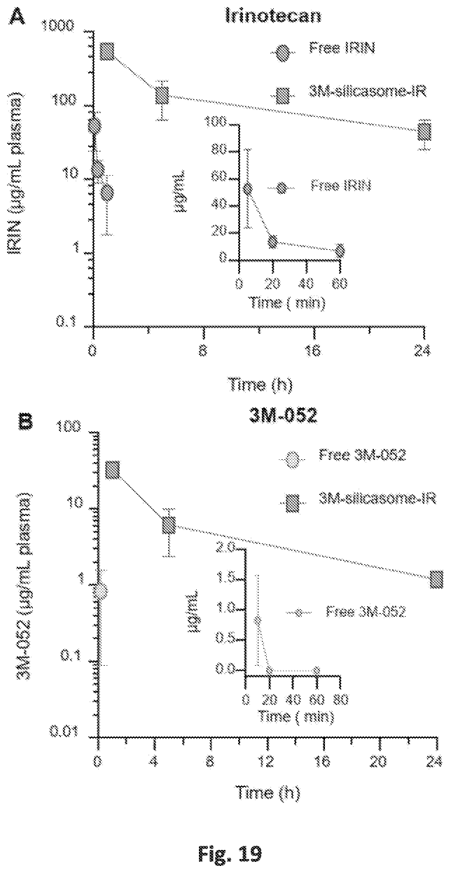

The pharmacokinetics (PK) studies were performed in healthy B6129SF1/J mice (n=3) which received a single IV injection of free 3M-052 (2 mg/kg), free IRIN (40 mg/kg), and 3M-silicasome-IR (3M-052, 2 mg/kg; IRIN, 40 mg/kg), respectively. Plasma samples were collected at different time points over 24 h and used to measure the concentrations of 3M-052 and IRIN by HPLC. These results show that while free 3M-052 and IRIN were rapidly cleared from the blood circulation, treatment with the dual-delivery carrier led to sustained increases in blood levels of both drugs for at least 24 h, confirming stable drug entrapment by the PEGylated LB (FIG. 19).

In order to assess the particle biodistribution, a DiR-labeled dual-delivery carrier (3M-silicasome-IR-DiR) was developed for IVIS imaging, as described in the Experimental Section. The particle characteristics are shown in FIG. 20, panel A. Biodistribution was assessed in the orthotopic KPC tumor model, derived by injecting stable luciferase-transfected KPC cells into the pancreas tails of female B6129SF1/J mice, as described by us.^{[7]} Orthotopic tumor-bearing mice (n=3-4) received a single IV injection of saline, free 3M-052 (2 mg/kg), free IRIN (40 mg/kg) and the DiR-labeled 3M-silicasome-IR (3M-052, 2 mg/kg; IRIN, 40 mg/kg), before collection of IVIS images (FIG. 20, panel B). In vivo IVIS imaging demonstrated effective biodistribution to the tumor site after 24 and 48 h (FIG. 20, panel C). However, since pancreas images overlap with the liver, we also performed animal sacrifice and organ explantation to collect ex vivo IVIS images and quantification of DiR fluorescence intensity. The right-side panel in FIG. 20, panel D shows representative images of an animal selected from each group, with the rest of the animal images on display in FIG. 28. Quantitative display of DiR intensities for both animal groups demonstrated 26.5% and 23.6% label distribution to the primary tumor sites after 24 or 48 h, respectively (FIG. 20, panel D). Comparable liver biodistribution values were 37.0% and 33.3%, respectively, in addition to some fluorescence intensity appearing in the spleens and intestines. The spleen, similar to lymph nodes, acts as a secondary lymphoid organ in supplementing immune responses in the pancreas.

We also used the orthotopic KPC model to assess the IRIN content at the primary tumor site, using HPLC analysis. These results demonstrate that treatment with the labeled 3M-silicasome-IR carrier could increase the IRIN content at the tumor site by 36- and 49-fold, respectively, after 24 and 48 h (FIG. 20, panel E). These results agree with the improved PK data in FIG. 19. It was not possible to quantify 3M-052 levels at the tumor site because the limited threshold of HPLC analysis for this substance.

### Immunotherapy Efficacy of Dual-Delivery Silicasomes in the Orthotopic KPC Tumor Model

While helpful to observe the chemoimmunotherapy response in subcutaneous KPC tumors, this tumor model displays a modest stromal content, differing from the extensive dysplasia seen in human PDAC. This is of relevance in considering the contribution of the stroma to shaping the immune-suppressive landscape in PDAC. While genetically engineered Pdx1-cre/LSL-Kras/G12D/p53R172H) mice do display many similarities to human PDAC (including a G12D KRAS mutation and TRP53 loss, extensive desmoplasia, and an immune suppressive TME), the logistical constraints of animal breeding, prompted the use of an orthotopic model, instead. We have previously shown that implantation of a luciferase-expressing KPC cell line into the pancreatic tail of immunocompetent B6129SF1/J mice can be used to obtain tumor growth, with retention of human PDAC features, including a more robust stroma that resemble the heterogeneous immune landscape of the genetic animal models.

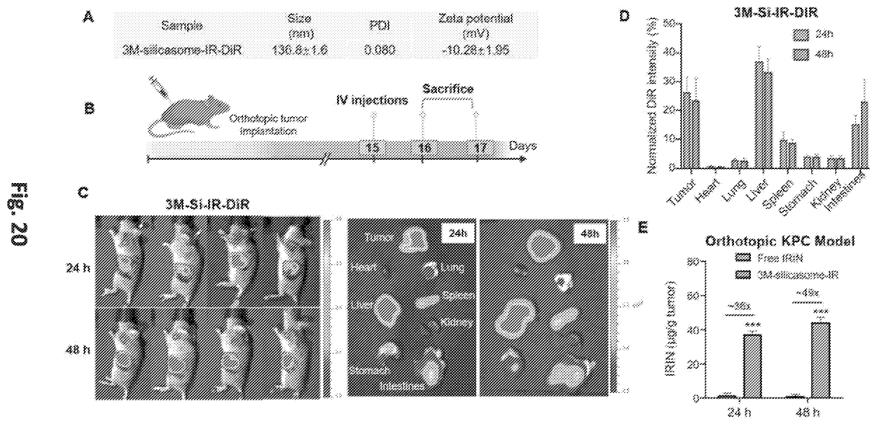

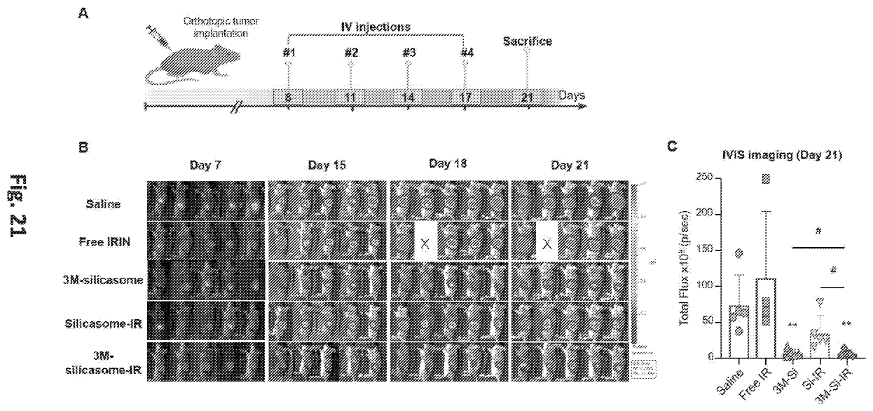

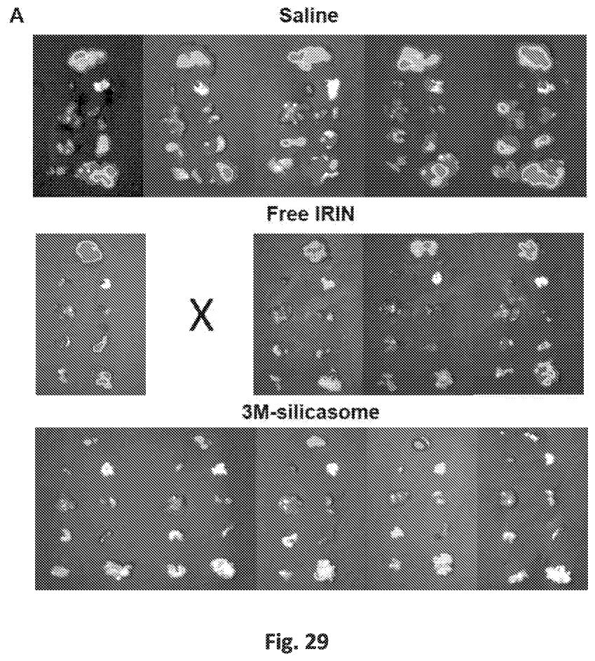

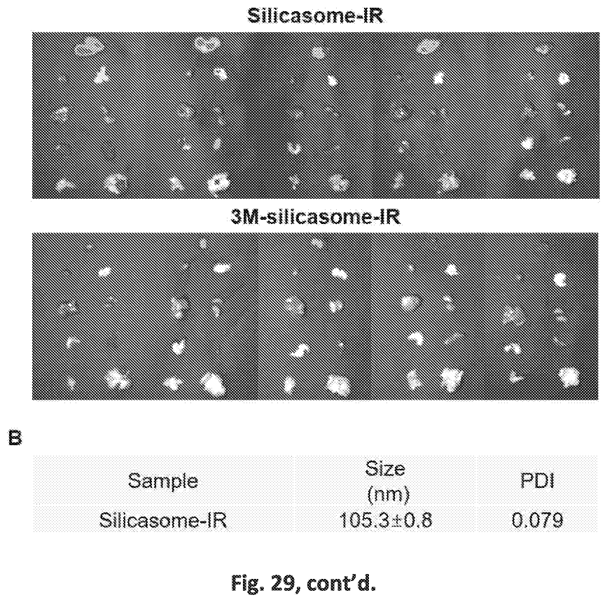

Orthotopic tumor-bearing B6129SF1/J female mice were established as described in FIG. 21, panel A. After confirming tumor growth at the pancreatic site by IVIS imaging, the tumor-bearing mice (n=5/6) received IV injection with saline, free IRIN (40 mg/kg), 3M-silicasome (3M-052, 2 mg/kg), silicasome-IR (IRIN, 40 mg/kg) and 3M-silicasome-IR (3M-052, 2 mg/kg; IRIN, 40 mg/kg) every 3 days for a total of 4 administrations (FIG. 20, panel A). Characterization of the IRIN-silicacasome (silicasome-IR or Si-IR) appear in FIG. 29, panel B. Animals continued to be monitored by in vivo IVIS imaging on days 7, 15, 18, and 21, as displayed in FIG. 21, panel B. Quantitative analysis of tumor bioluminescence intensity (“region of interest”) on day 21 demonstrated significant intensity decline in mice treated with the 3M-silicasome or 3M-silicasome-IR, compared to the saline control (FIG. 21, panel C). Moreover, the dual-drug silicasome had a statistically significant effect (p<0.05) compared with the single-drug silicasome. These findings were further corroborated by photographs, displaying tumor size (FIG. 21, panel D), in addition to conducting tumor weight assessment (FIG. 21, panel E).

Following animal sacrifice on day 21, primary tumors and organs were harvested for IVIS imaging and for performing IHC analysis of the tumor immune landscape. Of note, ex vivo IVIS image analysis showed differences in the level of metastatic spread to the liver, kidneys, stomach, spleen, and intestines among the different treatment modalities (FIG. 21, panel F). Thus, while there was evidence for splenic involvement in the representative example displayed in FIG. 21, panel F, for free IRIN, there was scant evidence of metastatic spread during treatment with single or dual drug silicasomes (FIG. 21, panel F). The comprehensive list of ex vivo IVIS images appears in FIG. 29, panel A.

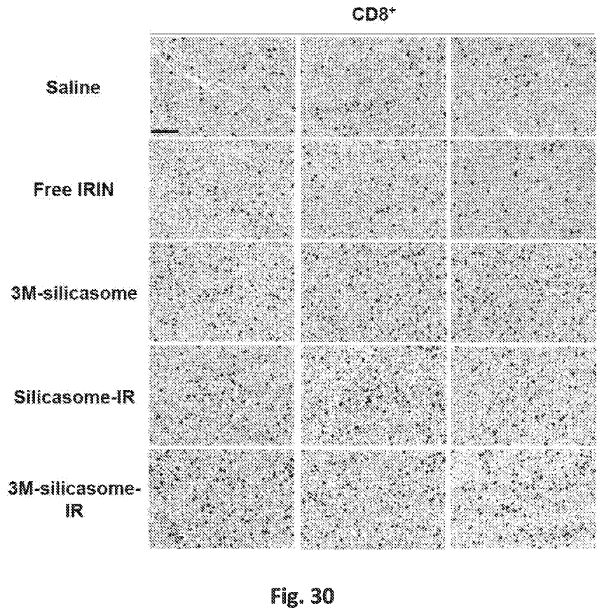

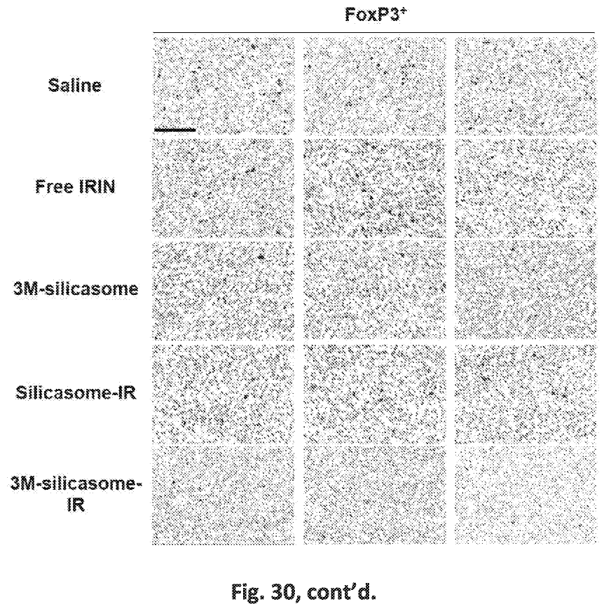

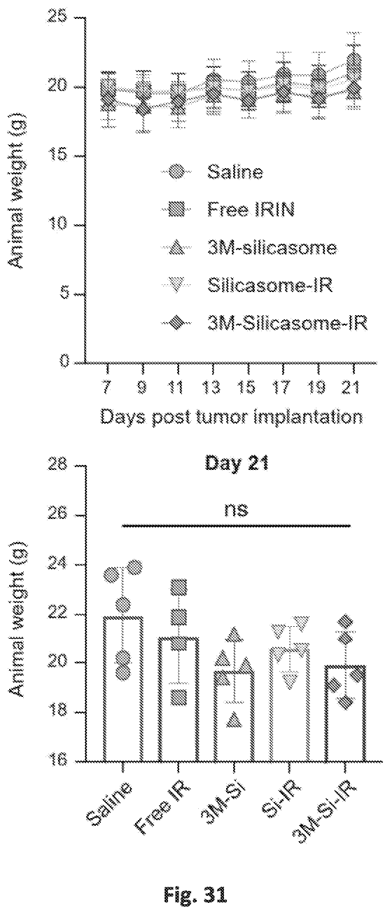

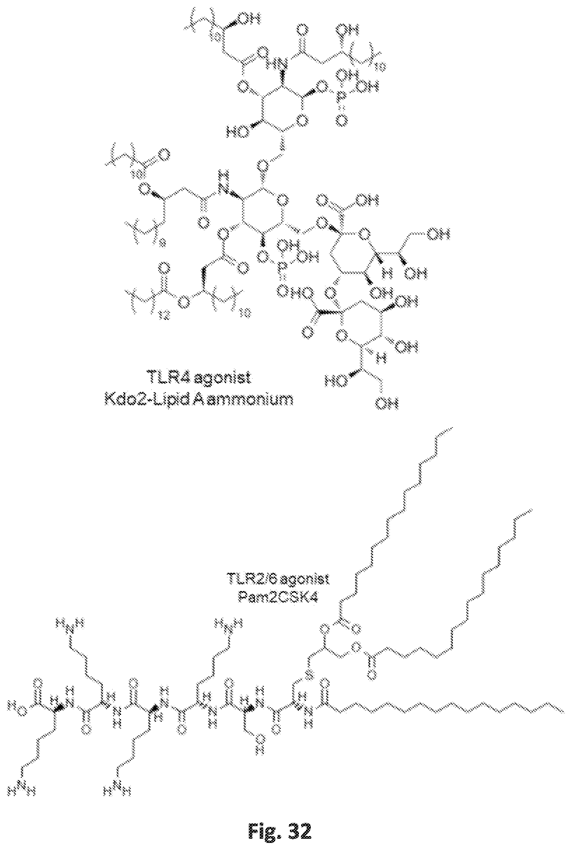

IHC analysis was performed to assess the expression of CD8⁺ T cells and FoxP3⁺ Treg cells, as demonstrated in FIG. 29, panel G. This demonstrate that the dual-drug silicasome showed the highest levels of CD8⁺ T-cell recruitment along with the largest decline in Treg cell numbers at the primary orthotopic tumor site (FIG. 29, panel G). This was also associated with the most statistically significant increase in the CD8⁺/Treg ratio compared to the other treatments (FIG. 29, panel G). Representative IHC images appear in FIG. 30. Body weight monitoring every 2 days did not show any significant weight loss among all groups (FIG. 31). There was also no evidence of toxicity elsewhere.

### DISCUSSION

In this study, we demonstrate that the CD18 lipid tail of the TLR7/8 agonist, 3M-052, can be incorporated into the lipid bilayer (LB) of a mesoporous silica nanocarrier (silicasome), which can also be used for remote loading of the amphipathic chemotherapeutic agent, irinotecan. We hypothesized that co-delivery of the TLR7/8 agonist with irinotecan may be able to mount a synergistic anti-PDAC immune response, based on boosting of APC function of dendritic cells receiving an enriched supply of tumor antigens as a result of the ICD response. We demonstrated effective TLR7 activation in a HEK-Blue™ mTLR7 reporter cell line, in addition to accomplishing activation and maturation of macrophage and BMDCs. This was followed by in vivo experimentation, demonstrating effective generation of an anti-PDAC immune response in a subcutaneous KPC tumor model, showing evidence of enhanced tumor shrinking, increased CD8⁺/Treg ratios and increased LNDC activation in response to the dual-delivery carrier. Moreover, further experimentation in a robust orthotopic KPC model confirmed the ability of the dual-delivery carrier to trigger a synergistic immune response, backed by data showing improved drug delivery and tumor biodistribution. Not only do these results demonstrate the efficacy of a co-formulated drug carrier but expand the scope of employing a chemoimmunotherapy approach for PDAC and other cancers.

One key advance demonstrated in this example is the impact of a co-delivered TLR7/8 agonist on a PDAC immune landscape, characterized by a paucity of neoantigens, poor antigen presentation, and interference in cytotoxic T-cell killing by a variety of immune escape or immune suppressive mechanisms. This includes dampening of the supply of conventional DC progenitors and their action as APC at the primary tumor site.^{[33]} This is also in agreement with the poor prognosis of PDAC patients expressing a reduced number of circulatory DC, thereby necessitating therapeutic intervention to improve APC supply and DC activation at the cancer site.^{[34]} While encapsulated irinotecan improves tumor antigen delivery to DC through its ICD effect, additional adjuvant stimuli are required for DC activation, maturation and APC function at the primary tumor as well as secondary lymphatic organ sites. It is therefore of major significance that the TLR family is widely expressed at the PDAC site and capable of improving DC function in response to danger signals. Moreover, a number of TLR agonists are available for therapeutic intervention, including synthetic imidazoquinoline agonists capable of ligating endosomal TLR7 and TLR8 receptors, including through silicasome delivery.

Protected delivery of TLR7/8 agonists provides an ideal opportunity for preventing inflammatory side effects, known to include manifestations such as pyrexia, fatigue, chills, decreased lymphocyte counts, nausea, or pain at the injection site.^{[20]} To overcome this problem localized administration of 3M-052 (MEDI-9197), either as a monotherapy or in combination with checkpoint blockade, has been attempted.^{[35]} However, in spite of minimal systemic effects in mouse models, severe systemic effects were still observed in a human phase I clinical trial, leading to abandonment of phase II trials.^{[20]} This triggered nanocarrier development, including by absorbing 3M-052 to lipid-decorated alum oxyhydroxide nanoparticles, used as an adjuvant during tuberculosis vaccination.^{[36]} Another approach was to encapsulate 3M-052 in PLGA nanoparticles, capable of providing even and more effective vaccination response to an HIV antigen than alum-TLR7.^{[28]} Moreover, Wightman et al. showed that liposomal encapsulation of 3M-052 could also serve as a vaccine adjuvant for antibody generation against viral proteins.^{[25]} While able to develop an in-house 3M-052 liposome (FIG. 22), we prefer to develop a carrier with a more stable supported LB, as previously demonstrated for obtaining toxicity reduction for a liposomal irinotecan carrier.^{[8]} We confirmed the safety of the silicasome carrier, showing no impact on animal weight or an impact on white blood cell counts, liver and kidney function, blood calcium, or phosphate (Table 10).

The efficacy of dual drug delivery by the 3M-silicasome-IR was demonstrated by more effective tumor shrinkage, compared to the response to the irinotecan- or 3M-silicasomes only (FIG. 25 panel B, and FIG. 29, panel C). Moreover, evidence was provided that this outcome is accomplished by immune activation, as demonstrated by the synergistic increase of CD8⁺/Treg ratios in animals treated with the dual-delivery compared to single-drug carriers (FIG. 18, panel A, and FIG. 29, panel G). Nonetheless, the 3M-silicasome had a significant effect on its own in the subcutaneous and orthotopic KPC tumor models (FIG. 25 and FIG. 29), demonstrating the utility of the TLR7/8 pathway on its own accord for PDAC. These results are also in agreement with the findings of Michaelis et al., who demonstrated effective tumor shrinkage, improved survival, and attenuation of cachexia in murine PDAC models treated with R848.^{[37]} This outcome was also accompanied by increased CD8⁺ T-cell infiltration and a reduction in the number of Tregs at the tumor site. Moreover, these authors also used a TLR knockout model to show that the major R848 impact was on a stromal TLR7-mediated immune response, rather than an impact on cancer cells.

Importantly, our subcutaneous KPC data demonstrate the importance of TLR7 on co-stimulatory receptor (CD80 and CD86) expression on lymph node-derived CD11c⁺ dendritic cells (FIG. 18, panel C). This agrees with the importance of lymph node participation in the cancer immunity cycle, including the irinotecan immunogenic response. Following calreticulin-mediated uptake of tumor antigens present in dying PDAC cells, participating DCs migrate to secondary lymphoid organs, where instruction of naïve T-cells allows activated, mature cytotoxic CD8⁺ T-cells to return to the tumor site. Not only do we demonstrate that encapsulated 3M-052 delivery is capable of boosting DC activation at the site and regional lymph nodes in the subcutaneous model, but also that the DiR-labeled 3M-Si-IR biodistributes to the spleen (FIG. 20, panels C and D). This finding is commensurate with the demonstration that conjugation of a TLR7 small-molecule, 1V209, to cholesterol could achieve construction of a liposome, capable of distributing to lymph nodes in CT26 colorectal cancer, 4T1 breast cancer, and Pan02 pancreatic ductal cancer models.^{[29]} Moreover, the encapsulation of resiquimod (R848) in PLA nanoparticles was capable of regional lymph node targeting and DC uptake to enhance immunotherapy for skin cancer.^{[38]}

In addition to impacting DC function, there are other cell types in the PDAC TME that could be impacted by TLR7 agonists. For instance, it has been shown that R848 reduces immune suppression by inducing differentiation of MDSC into macrophages and DCs.^{[39]} The same applies to tumor-associated macrophages, including demonstration that R848 and 3M-052 are potent drivers of the M1 differentiation, including during nanoparticles delivery.^{[40]} In addition to additional cell targets, it is appropriate to consider combining TLR7/8 with other TLR agonists, including CpG oligonucleotides, polyinosinic-polycytidylic acid, monophosphoryl 3-deacyl lipid A (3D-PHAD or 3D(6-acyl)-PHAD) for activation of TLR9, TLR3 and TLR4, respectively.^{[20]} Moreover, several of these agonists are available as lipophilic agents for silicasome LB incorporation. This is in agreement with the demonstration that a TLR combination strategy can lead to synergistic DC activation, e.g., combining R848 with either a TLR4 agonist or a TLR3 agonist to activate multiple human DC subsets.^{[41]} It is also possible to combine TLR7/8 agonists with other classes of immune-modulatory agents, such as the combined use with antibodies to checkpoint receptors, EGFR, HER2/neu or OX-40, as well as photothermal therapy.^{[20]}

### CONCLUSIONS

In summary, we developed a synergistic drug carrier to co-deliver a chemotherapeutic agent with a TLR7/8 agonist for augmenting the ICD-inducing effect of irinotecan by the DC activating effect of 3M-052, including at a participating lymph node venue. Based on the fact that we have already achieved upscale manufacturing of a single drug silicasome platform, these results hold great promise for strengthening this platform for use in the treatment of PDAC and other cancers. Where necessary, combination therapy can also be achieved by synthesizing liposomes that combine irinotecan and 3M-052. Moreover, it is also possible to load other ICD-inducing chemotherapeutic agents (e.g., doxorubicin, mitoxantrone, oxaliplatin) into these carriers, where the chemoimmunotherapy effects can be combined with co-delivered TLR agonists, small-molecule PD1 inhibitors, GSK3 inhibitors and TGF-beta inhibitors to intervene in multiple cancer types.

### Experimental Section

### Materials

1,2-distearoyl-sn-glycero-3-phosphocholine (DSPC), cholesterol (Chol) and 1,2-distearoyl-sn-glycero-3-phosphoethanolamine-N-[methoxy(polyethylene glycol)-2000](ammonium salt) (DSPE-PEG2000) were purchased from Avanti Polar Lipids, USA. Fetal bovine serum (FBS) was purchased from Gemini Bio Products. Dulbecco's modified Eagle medium (DMEM), Roswell Park Memorial Institute (RPMI) 1640 medium, penicillin, streptomycin, DiD′ solid (DiIC₁₈(5) solid (1,1′-Dioctadecyl-3,3,3′,3′-Tetramethylindodicarbocyanine, 4-Chlorobenzenesulfonate Salt)) and DiR′ (DiIC₁₈(7) (1,1′-Dioctadecyl-3,3,3′,3′-Tetramethylindotricarbocyanine Iodide)) were purchased from Invitrogen. 3M-052 (a.k.a. Telratolimod) and Cell Counting Kit-8 (CCK-8) were purchased from MedChemExpress, USA. Irinotecan hydrochloride trihydrate was purchased from LC Laboratories, USA. ACK Lysing Buffer was purchased from Thermo Fisher Scientific Inc., USA. HEPES (4-(2-hydroxyethyl)-1-piperazineethanesulfonic acid), dextrose, DNase I, and Collagenases (Type II and IV) were purchased from Sigma-Aldrich, USA. Recombinant Murine granulocyte-macrophage colony-stimulating factor (GM-CSF) was purchased from PeproTech, USA. ELISA kits for quantitative measurement of murine IL-12/IL-23 p40 and TNF-alpha levels were purchased from R&D Systems, Inc., USA. The murine assay for assessing TLR-induced reporter gene activity in HEK 293 cells (HEK-Blue™ Detection) as well as R848 (Resiquimod) were purchased from InvivoGen, USA. Anti-mouse CD16/32 antibody, Zombie Violet™, Cell Staining Buffer, FITC anti-mouse CD11c antibody, anti-mouse CD80 antibody, PerCP/Cyanine5.5 anti-mouse CD45.2 antibody, anti-mouse CD11c antibody, PE anti-mouse CD80 antibody, and FITC anti-mouse CD86 antibody were purchased from BioLegend, USA.

### Synthesis, Purification, and Characterization of 3M-Silicasome-IR

Bare MSNPs were synthesized and purified by extensive acidic ethanol washing to remove the CTAC detergent, previously described by us.^{[8]} The trapping agent, TEA₈SOS, was prepared from a commercially available sucrose octasulfate (SOS) sodium salt through an ion-exchange chromatography procedure, previously described by us.^{[7]} For the synthesis of 3M-silicasome, a mixture of 50 mg of 3M-052 and lipids (DSPC/Chol/DSPE-PEG2000/3M-052, in the molar ratio of 55.5:38.5:2.7:3.3) was dissolved in 0.1 mL pure ethanol at 65° C. Then, 1 mL of a preheated (65° C.) solution, containing 40 mg MSNP soaked in 80 mM TEA₈SOS trapping agent, was poured into the lipid solution and mixed with a pipette. This mixture was treated by probe sonication with a 15/15 s on/off working cycle and a power output of 32.5 W. After 15 min, the sample was purified by size-exclusion chromatography, using a Sepharose CL-4B column with a HEPES (5 mM HEPES, 5% dextrose, pH 6.5) buffer elution to remove the free trapping agent.

For IRIN remote loading, 20 mg irinotecan was dissolved in 2 mL HEPES-buffered dextrose, before mixing and incubation of the TEA₈SOS-loaded 3M-silicasome at 65° C. for 30 min. The reaction was quenched on ice water for another 30 min, following which the irinotecan-loaded silicasome, designated 3M-silicasome-IR, was washed three times by centrifugation at 15 000 rpm for 30 min in a HEPES-buffered NaCl solution (4.05 mg/mL HEPES, 8.42 mg/mL NaCl, pH 7.2). In addition to the synthesis of dual-delivery particles, we also constructed particles capable of delivering IRIN only by eliminating 3M from the lipid suspension.

In experiments requiring particles for the performance of in vitro and in vivo imaging, DiD and DiR labeling was performed by adding 0.2 mg DiD or DiR into the mixture of 50 mg of 3M-052 and lipids (DSPC/Chol/DSPE-PEG2000/3M-052, in the molar ratio of 55.5:38.5:2.7:3.3). The procedure used for DiD-3M-silicasome or DiR-3M-silicasome-IR was the same as that of 3M-silicasome-IR.

For the characterization of the nanocarriers, loading capacity was determined by calculating the weight ratio of 3M-052 or IRIN relative to the total particle composition. MSNP mass was determined by TGA. The concentrations of 3M-052 and IRIN were determined by the UV-vis absorbance at 320 nm and 360 nm respectively (M5e, Molecular Device, USA). Particle hydrodynamic size, size distribution, and zeta potential were measured by a ZETAPALS instrument (Brookhaven Instruments Corporation). The uniformity and integrity of the lipid-coated particles containing 3M-052, with or without IRI remote loading, were characterized by the performance of cryoEM, using a TF20 FEI Tecnai-G2 instrument.

### Cell Culture

KRAS transformed murine pancreatic adenocarcinoma (KPC) cells, derived from a spontaneous tumor originating in a transgenic KrasLSL-G12D/+; Trp53LSL-R172H/+; Pdx-1-Cre mouse model, was used for stable transfection with a luciferase-based lentiviral vector to derive KPC-luc cells. The cells as well as the murine macrophage-like cell line (RAW264.7) cells were cultured in DMEM with 10% (v/v) FBS, 100 units/mL of penicillin, and 100 mg/mL of streptomycin under 37° C. with 5% CO₂.

Bone marrow-derived dendritic cells (BMDCs) were prepared according to our established procedure, with a slight modification.^{[42]} Bone marrow cells flushed from the femur and tibia of B6/129 mice were collected and red blood cells were lysed by incubating in ACK Lysing Buffer. On the 1St day, cells were cultured in a 24-well plate with 1 mL/well of RPMI-1640 medium supplemented with 10% (v/v) FBS, 100 units/mL of penicillin, 100 mg/mL of streptomycin and 20 ng/mL GM-CSF under 37° C. with 5% CO₂. On the 3^{rd} and 5^{th} day, the culture medium was replaced to include GM-CSF for an additional 2 days. Immature BMDCs were acquired by using non-adherent or loosely adherent cells for centrifugation at 1400 rpm for 5 min.

Commercially acquired HEK-Blue™ mTLR7 cells, derived from the human embryonic kidney HEK293 cell line, were used to confirm the generation of TLR7 signaling by free and encapsulated 3M-052, demonstrating NF-κB/AP1-induced activation of the transgene promotor leads to release of secreted embryonic alkaline phosphatase (SEAP). These cells were cultured in DMEM medium supplemented with 10% (v/v) FBS, 100 units/mL of penicillin, 100 mg/mL of streptomycin, and 10 μg/ml of Blasticidin, and 100 μg/ml of Zeocin.

### Cellular Response of TLR7 Reporter Cell Line

A suspension of 2.2×10⁵ cells/mL was prepared in HEK-Blue™ Detection medium, following which 180 μL aliquots (around 4×10⁴ cells/well) were dispensed into the wells of a 96-well plate. This was followed by the addition of 20 μL PBS (negative control), R848 (positive control), free 3M-052, and 3M-silicasome to achieve a concentration range of 0.01 to 10 μM. Cells were incubated at 37° C. in 5% CO₂ for 20 h. The release of SEAP into the supernatant was determined, using a microplate reader at 630 nm.

### Assessment of RAW264.7 and BMDC Responses to TLR7 Activation

RAW264.7 cells (1×10⁵ cells/well) were cultured in 48-well plates for 24 h, before the addition of PBS or 10 μM concentrations of R848, free 3M-052 or the 3M-silicasome for a further 21 h. Immature BMDCs (5×10⁵ cells/well) were cultured in 24-well plates, receiving the same dose of R848, free 3M-052, and 3M-silicasome for 21 h. The cellular suspensions from each well were collected and centrifuged to obtain supernatants for ELISA analysis. This included measurement of murine IL-12p40 and TNF-α levels, using each vendor's protocol.

For flow cytometry analysis of surface markers, cells were washed with cell staining buffer, before staining RAW264.7 cells with anti-CD80 or BMDCs with FITC-conjugated anti-CD11c and anti-CD80 on ice, according to the manufacturer's instructions. The assessment of the differentiation and activation markers were evaluated by flow cytometry (LSRFortessa; BD Biosciences), analyzed by FlowJo software.

### Cell Viability Test, Cellular Uptake and In Vitro Killing Effects

For cell viability testing, KPC and RAW264.7 cells were seeded in 100 μL culture medium in 96-well plates at a density of 3-5×10³ cells per well for 24 h, before the addition of 100 μL fresh medium containing the 3M-silicasome at 3M-052 concentrations of 2, 4, 8, 16, 20, 30, 40 μM for a further 48 h. After removal, MTS was added in fresh medium at a concentration of 317 μg/mL for an additional 1 to 2 h, before the determination of UV-visible absorption at 490 nm in a microplate reader. Cell viability (%) was calculated using the formula: (ODₛₐₘₚₗₑ−OD_{blank})/(OD_{control}−OD_{blank})×100.

To assess cellular nanocarrier uptake, the DiD-3M-silicasome was incubated with RAW264.7 cells (1×10⁵ cells/well) to deliver concentrations of 2, 5, and 10 μM for 21 h. The same procedure was followed for immature BMDCs (5×10⁵ cells/well), incubated with the same concentration range of the DiD-3M-silicasome for 21 h in a 24-well plate. The cells were harvested and washed before assessing Di D fluorescence in a flow cytometer, using FlowJo software.

To verify KPC cytotoxicity, cells seeded in 96-well plates were incubated with different concentrations (50, 100, 200, 300, 400, and 500 μM) of free IRIN and the 3M-silicasome-IR for 48 h. 20 μL of CCK-8 solution was added to each well and the plates were incubated for an additional 1-2 h in an incubator. The absorbance of each well was assessed at 450 nm in a microplate reader. Cytotoxicity (% dead cells) was calculated using the formula: (ODₛₐₘₚₗₑ−OD_{blank})/(OD_{control}−OD_{blank})×100.

### Assessment of 3M-052 and IRIN Pharmacokinetics

All animal experimental protocols were approved by the UCLA Animal Research Committee. The PK study was carried out in 8-10-week-old healthy female B6129SF1/J mice which received one IV injection of free 3M-052, free IRIN, and 3M-silicasome-IR at dose equivalents of 2 mg/kg and 40 mg/kg for 3M-052 and IRIN, respectively. Blood collections were performed at 1, 5, 24, and 48 h post-injection, and the plasma was collected by centrifuging at 3500 rpm in plasma collecting tubes. Free 3M-052 and IRIN were extracted by methanol and measured by HPLC. The details are described in the HPLC analysis section.

### Assessment of Carrier Efficacy, Using a Subcutaneous KPC Tumor Model

A subcutaneous KPC tumor model was established in immunocompetent B6129SF1/J mice, using protocols approved by the UCLA Animal Research Committee. Briefly, 100 μL of a DMEM/Matrigel (1:1 v/v) suspension, containing 1×10⁶ KPC cells, was subcutaneously injected into the right flank of female B6129SF1/J mice, 8-10 weeks old. Tumor-bearing mice were randomly assigned into 5 groups (n=6-7), which received IV injections of saline, free 3M-052, free IRIN, 3M-silicasome, and 3M-silicasome-IR, respectively, when tumor sizes reached ˜100 mm³. Injections were given every 3-4 days, using the dosing schedule that delivers the equivalent of 2 mg/kg and 40 mg/kg, respectively, for 3M-052 and IRIN. Subcutaneous KPC tumor size and animal weight were monitored every 2 days. The tumor size was calculated according to the formula: (Width²×Length)/2.

Following animal sacrifice, photographs were obtained of the harvested tumors, which were subsequently fixed in 10% formalin for IHC staining by the UCLA Translational Pathology Core Laboratory. Image processing was performed using Aperio ImageScope software (Leica). IHC analysis was performed to assess tumor staining intensity for CD8 and FoxP3. Additionally, we assessed the immune response in the draining inguinal lymph nodes (LNs) harvested from the sacrificed animals. The lymph nodes were washed in PBS, before slicing into small fragments. These fragments were placed into a 6-well plate, incubated in 5 mL digestion medium (1 mg/mL collagenase, type II, 1 mg/mL collagenase, type IV and 0.2 mg/mL DNase, Type I) at 37° C. for 2-3 h. Single-cell suspensions were obtained by passing the tissue digests through a 70 mm cell strainer, followed by washing in a cell staining buffer. For the performance of flow cytometry analysis, cells were stained on ice with PerCP/Cyanine5.5-CD45 and anti-CD11c antibodies to analyze the number of activated DCs, in addition to the use of PE-CD80 and FITC-CD86 antibodies to assess DC maturation. The flow cytometry procedure for the analysis of these phenotypes, with the use of FlowJo software, is explained in FIG. 26.

We also collected blood for separating serum after centrifugation at 3500 rpm for 20 min. The following biochemical parameters were assayed by UCLA Division of Laboratory Animal Medicine (DLAM) diagnostic laboratory services: white blood cell (WBC), alkaline phosphatase (ALP), aspartate aminotransferase (AST), alanine aminotransferase (ALT), blood urea nitrogen (BUN), creatinine, calcium, and phosphorus.

### Assessment of Nanocarrier and Drug Biodistribution in an Orthotopic KPC-Luc Tumor Model

A DiR-labeled 3M-silicasome-IR nanocarrier was prepared for biodistribution assessment in a KPC-derived orthotopic tumor model in immunocompetent B6129SF1/J mice, as previously described by us^{[7]}. The animal protocol received institutional approval. Briefly, a 50 μL suspension of DMEM/Matrigel (6:4 v/v), containing 8×10⁵ KPC-luc cells, was injected into the pancreas tail of female B6129SF1/J mice, using a limited surgical procedure under anesthesia. After 15 days, tumor-bearing mice were randomly assigned into 5 treatment groups (n=3-4), designed to receive one IV injection of saline, free IRIN, and DiR-3M-silicasome-IR. The dose equivalent for IRIN was 40 mg/kg. The in vivo fluorescence intensity of the DiR label group was performed in a Xenogen IVIS imaging system 24 and 48 h after IV injection. Following animal sacrifice, tumors and major organs were harvested from the DiR-3M-silicasome-IR treated group, and also used for ex vivo IVIS imaging and quantitative analysis. In addition, tumors were also collected from all groups to determine IRIN contents by HPLC analysis.

### Assessment of the Therapeutic Efficacy in the Orthotopic KPC-Luc Tumor Model

50 μL of a DMEM/Matrigel (6:4 v/v) suspension, containing 8×10⁵ KPC-luc cells, was injected into the tail of the pancreas in female B6129SF1/J mice by a survival surgery procedure. One week after the surgery, IVIS imaging was used to confirm the establishment of bioluminescent tumors, following which animals were randomly assigned into 5 treatment groups (n=5), earmarked for IV injection of saline, free IRIN, 3M-silicasome, silicasome-IR, and 3M-silicasome-IR, respectively, every 3 days. The dose equivalents of 3M-052 and IRIN were 2 mg/kg and 40 mg/kg per dose, respectively. Animal weight was monitored every 2 days.

To perform the bioluminescence imaging of the luciferase-expressing tumors, mice were injected intraperitoneal with 50 mg/kg D-Luciferin on day 7, 15, 18, and 21. Ex vivo IVIS images of tumors and major organs, collected from sacrificed animals, were obtained on day 21. In addition, we also obtained photographs and weighed the harvested tumors, which were subsequently fixed in 10% formalin for the performance of IHC staining. IHC analysis was the same as described above.

### HPLC Analysis

The harvested tumor and organ samples, collected in the drug biodistribution experiment, were weighed and homogenized. The collected plasma, tumor, and organ homogenates were extracted in methanol, using 1:5, 1:4, and 1:3 v/v dilutions, respectively. The extracts were vortexed for 20 s and centrifuged at 13000 rpm for 10 min, following which the drug-containing supernatants were filtrated through 0.22 μm filters to perform HPLC analysis. The HPLC system is operated by a Knauer Smartline Pneumatic Pump, C18 column, K-2600 spectrophotometer, and Gina data acquisition software. The mobile phase consisted of mobile phase A (0.01% trifluoroacetic acid in water) and mobile phase B (0.01% trifluoroacetic acid in methanol) as 70% A and 30% B (v/v). A 20 μL of the sample was injected to measure the 3M-052 and IRIN absorptions at 320 and 360 nm. The 3M-052 and IRIN standard curves were generated over the maximal concentrations of 40 and 100 μg/mL respectively.

### Statistical Analysis

Differences among groups were estimated by one-way ANOVA analysis. Data were expressed as mean±standard deviation (S.D.), representing at least three independent experiments. A statistically significant difference was considered at *p<0.05; **p<0.01; ***p<0.001; #p<0.05; ##p<0.01 and ###p<0.001, as indicated in the figure legends.

### REFERENCES FOR EXAMPLE 3

[1] R. L. Siegel, K. D. Miller, H. E. Fuchs, A. Jemal, CA: A Cancer Journal for Clinicians 2021, 71, 7.
[2] R. L. Siegel, K. D. Miller, H. E. Fuchs, A. Jemal, CA: A Cancer Journal for Clinicians 2022, 72, 7.
[3] G. C. Chu, A. C. Kimmelman, A. F. Hezel, R. A. DePinho, Journal of Cellular Biochemistry 2007, 101, 887.
[4] A. Wang-Gillam, C.-P. Li, G. Bodoky, A. Dean, Y.-S. Shan, G. Jameson, T. Macarulla, K.-H. Lee, D. Cunningham, J. F. Blanc, R. A. Hubner, C.-F. Chiu, G. Schwartsmann, J. T. Siveke, F. Braiteh, V. Moyo, B. Belanger, N. Dhindsa, E. Bayever, D. D. Von Hoff, L.-T. Chen, N.-S. Group, The Lancet 2016, 387, 545.
[5] J. E. Frampton, Drugs 2020, 80, 1007.
[6] H. Meng, M. Wang, H. Liu, X. Liu, A. Situ, B. Wu, Z. Ji, C. H. Chang, A. E. Nel, ACS Nano 2015, 9, 3540.
[7] X. Liu, A. Situ, Y. Kang, K. R. Villabroza, Y. Liao, C. H. Chang, T. Donahue, A. E. Nel, H. Meng, ACS Nano 2016, 10, 2702.
[8] X. Liu, J. Jiang, R. Chan, Y. Ji, J. Lu, Y.-P. Liao, M. Okene, J. Lin, P. Lin, C. H. Chang, X. Wang, I. Tang, E. Zheng, W. Qiu, Z. A. Wainberg, A. E. Nel, H. Meng, ACS Nano 2019, 13, 38.
[9] X. Liu, J. Jiang, Y.-P. Liao, I. Tang, E. Zheng, W. Qiu, M. Lin, X. Wang, Y. Ji, K.-C. Mei, Q. Liu, C. H. Chang, Z. A. Wainberg, A. E. Nel, H. Meng, Advanced Science 2021, 8, 2002147.
[10] O. Kepp, L. Menger, E. Vacchelli, C. Locher, S. Adjemian, T. Yamazaki, I. Martins, A. Q. Sukkurwala, M. Michaud, L. Senovilla, L. Galluzzi, G. Kroemer, L. Zitvogel, Cytokine & Growth Factor Reviews 2013, 24, 311.
[11] L. Bezu, L. C. Gomes-da-Silva, H. Dewitte, K. Breckpot, J. Fucikova, R. Spisek, L. Galluzzi, O. Kepp, G. Kroemer, Frontiers in Immunology 2015, 6.
[12] G. Kroemer, L. Galluzzi, O. Kepp, L. Zitvogel, Annu. Rev. Immunol. 2013, 31, 51.
[13] L. Galluzzi, A. Buqu6, O. Kepp, L. Zitvogel, G. Kroemer, Nat Rev Immunol 2017, 17, 97.
[14] D. S. Chen, I. Mellman, Immunity 2013, 39, 1.
[15] A. E. Nel, K.-C. Mei, Y.-P. Liao, X. Liu, ACS Nano 2022, 16, 5184.
[16]S. Akira, K. Takeda, T. Kaisho, Nat Immunol 2001, 2, 675.
[17] O. Takeuchi, S. Akira, Cell 2010, 140, 805.
[18] K. Takeda, S. Akira, International Immunology 2005, 17, 1.
[19] F. Leulier, B. Lemaitre, Nat Rev Genet 2008, 9, 165.
[20] S. Bhagchandani, J. A. Johnson, D. J. Irvine, Advanced Drug Delivery Reviews 2021, 175, 113803.
[21] A. Iwasaki, R. Medzhitov, Nat Immunol 2004, 5, 987.
[22] J. Ryu, F. C. Yang, Clinical Medicine. Therapeutics 2009, 1, CMT.S1969.
[23] U. Hengge, B. Benninghoff, T. Ruzicka, M. Goos, The Lancet Infectious Diseases 2001, 1, 189.
[24] A. L. Engel, G. E. Holt, H. Lu, Expert Review of Clinical Pharmacology 2011, 4, 275.
[25] D. Smirnov, J. J. Schmidt, J. T. Capecchi, P. D. Wightman, Vaccine 2011, 29, 5434.
[26] Q. Wang, M. A. Barry, C. A. Seid, E. M. Hudspeth, C. P. McAtee, M. J. Heffernan, Journal of Biomedical Materials Research Part B: Applied Biomaterials 2018, 106, 1587.
[27] F. Auderset, E. Belnoue, B. Mastelic-Gavillet, P.-H. Lambert, C.-A. Siegrist, Frontiers in Immunology 2020, 11.
[28] S. P. Kasturi, M. A. U. Rasheed, C. Havenar-Daughton, M. Pham, T. Legere, Z. J. Sher, Y. Kovalenkov, S. Gumber, J. Y. Huang, R. Gottardo, W. Fulp, A. Sato, S. Sawant, S. Stanfield-Oakley, N. Yates, C. LaBranche, S. M. Alam, G. Tomaras, G. Ferrari, D. Montefiori, J. Wrammert, F. Villinger, M. Tomai, J. Vasilakos, C. B. Fox, S. G. Reed, B. F. Haynes, S. Crotty, R. Ahmed, B. Pulendran, Science Immunology 2020, 5, eabb1025.
[29] D. Wan, H. Que, L. Chen, T. Lan, W. Hong, C. He, J. Yang, Y. Wei, X. Wei, Nano Lett. 2021, 21, 7960.
[30] N. Hiraoka, K. Onozato, T. Kosuge, S. Hirohashi, Clinical Cancer Research 2006, 12, 5423.
[31] M. Wartenberg, I. Zlobec, A. Perren, V. H. Koelzer, B. Gloor, A. Lugli, K. Eva, Oncotarget 2015, 6, 4190.
[32] R. Saleh, E. Elkord, Cancer Letters 2020, 490, 174.
[33] S. Hegde, V. E. Krisnawan, B. H. Herzog, C. Zuo, M. A. Breden, B. L. Knolhoff, G. D. Hogg, J. P. Tang, J. M. Baer, C. Mpoy, K. B. Lee, K. A. Alexander, B. E. Rogers, K. M. Murphy, W. G. Hawkins, R. C. Fields, C. J. DeSelm, J. K. Schwarz, D. G. DeNardo, Cancer Cell 2020, 37, 289.
[34] M. A. Meyer, J. M. Baer, B. L. Knolhoff, T. M. Nywening, R. Z. Panni, X. Su, K. N. Weilbaecher, W. G. Hawkins, C. Ma, R. C. Fields, D. C. Linehan, G. A. Challen, R. Faccio, R. L. Aft, D. G. DeNardo, Nat Commun 2018, 9, 1250.
[35] S. R. Mullins, J. P. Vasilakos, K. Deschler, I. Grigsby, P. Gillis, J. John, M. J. Elder, J. Swales, E. Timosenko, Z. Cooper, S. J. Dovedi, A. J. Leishman, N. Luheshi, J. Elvecrog, A. Tilahun, R. Goodwin, R. Herbst, M. A. Tomai, R. W. Wilkinson, Journal for ImmunoTherapy of Cancer 2019, 7, 244.
[36] C. B. Fox, M. T. Orr, N. Van Hoeven, S. C. Parker, T. J. T. Mikasa, T. Phan, E. A. Beebe, G. I. Nana, S. W. Joshi, M. A. Tomai, J. Elvecrog, T. R. Fouts, S. G. Reed, Journal of Controlled Release 2016, 244, 98.
[37] K. A. Michaelis, M. A. Norgard, X. Zhu, P. R. Levasseur, S. Sivagnanam, S. M. Liudahl, K. G. Burfeind, B. Olson, K. R. Pelz, D. M. Angeles Ramos, H. C. Maurer, K. P. Olive, L. M. Coussens, T. K. Morgan, D. L. Marks, Nat Commun 2019, 10, 4682.
[38] J. Widmer, C. Thauvin, I. Mottas, V. N. Nguyen, F. Delie, E. Allémann, C. Bourquin, International Journal of Pharmaceutics 2018, 535, 444.
[39] M. Lee, C.-S. Park, Y.-R. Lee, S.-A. Im, S. Song, C.-K. Lee, Arch. Pharm. Res. 2014, 37, 1234.
[40] C. B. Rodell, S. P. Arlauckas, M. F. Cuccarese, C. S. Garris, R. Li, M. S. Ahmed, R. H. Kohler, M. J. Pittet, R. Weissleder, Nat Biomed Eng 2018, 2, 578.
[41] G. Napolitani, A. Rinaldi, F. Bertoni, F. Sallusto, A. Lanzavecchia, Nat Immunol 2005, 6, 769.
[42] K. Inaba, M. Inaba, N. Romani, H. Aya, M. Deguchi, S. Ikehara, S. Muramatsu, R. M. Steinman, Journal of Experimental Medicine 1992, 176, 1693.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A drug delivery vehicle for the co-delivery of a chemotherapeutic agent and a Toll-Like Receptor (TLR) agonist and/or a lipoxin, said vehicle comprising:
a silicasome comprising:
a porous nanoparticle encapsulated in a lipid bilayer, where:
said lipid bilayer contains a lipoxin and/or a lipid compatible Toll-Like Receptor (TLR) agonist disposed in the lipid bilayer; and
said chemotherapeutic agent is contained in pores comprising said porous nanoparticle and said chemotherapeutic agent comprises a chemotherapeutic agent that induces immunogenic cell death (ICD); or
a liposome comprising a lipid bilayer where:
said lipid bilayer contains a lipoxin and/or a lipid compatible a Toll-Like Receptor (TLR) agonist disposed in the lipid bilayer; and
said chemotherapeutic agent is inside said liposome and said chemotherapeutic agent comprises a chemotherapeutic agent that induces immunogenic cell death (ICD).

2. The drug delivery vehicle of claim 1, wherein said TLR agonist comprises a TLR7/8 agonist.

3. The drug delivery vehicle according to any one of claims 1-2, wherein said vehicle comprises a silicasome.

4. The drug delivery vehicle of claim 3, wherein said porous nanoparticle comprises a mesoporous silica nanoparticle.

5. The drug delivery vehicle according to any one of claims 1-2, wherein said vehicle comprises a liposome.

6. The drug delivery vehicle according to any one of claims 1-5, wherein said drug delivery vehicle comprises as lipid compatible TLR agonist.

7. The drug delivery vehicle of claim 6, wherein said drug delivery vehicle comprises a lipidated TLR agonist.

8. The drug delivery vehicle according to any one of claims 6-7, wherein said drug delivery vehicle comprises a TLR7/TLR8 agonist.

9. The drug delivery vehicle of claim 8, wherein said drug delivery vehicle comprises a lipidated TLR7/8 agonist selected from the group consisting of 3M-052, lipidated UM-3001, and an imidazoquinoline molecule covalently linked to a phospho- or phosphonolipid group.

10. The drug delivery vehicle of claim 9, wherein said lipidated TLR7/8 agonist comprises 3M-052 (Telratolimod).

11. The drug delivery vehicle of claim 9, wherein said lipidated TLR7/8 agonist comprises a lipidated UM-3001 selected from the group consisting of UM-3003, UM-3004, and UM-3005.

12. The drug delivery vehicle of claim 11, wherein said lipidated imidazoquinoline comprises UM-3003.

13. The drug delivery vehicle of claim 11, wherein said lipidated imidazoquinoline comprises UM-3004.

14. The drug delivery vehicle of claim 11, wherein said lipidated imidazoquinoline comprises UM-3005.

15. The drug delivery vehicle of claim 9, wherein said lipidated TLR7/8 agonist comprises a lipidated imidazoquinoline a molecule selected from the group consisting of L1, L2, L3, L4, and L5.

16. The drug delivery vehicle of claim 15, wherein said lipidated imidazoquinoline comprises L1.

17. The drug delivery vehicle of claim 15, wherein said lipidated imidazoquinoline comprises L2.

18. The drug delivery vehicle of claim 15, wherein said lipidated imidazoquinoline comprises L3.

19. The drug delivery vehicle of claim 15, wherein said lipidated imidazoquinoline comprises L4.

20. The drug delivery vehicle of claim 15, wherein said lipidated imidazoquinoline comprises L5.

21. The drug delivery vehicle according to any one of claims 1-20, wherein said drug delivery vehicle comprises a lipoxin.

22. The drug delivery vehicle of claim 21, wherein said lipoxin comprises LXA4 or an analog thereof.

23. The drug delivery vehicle of claim 22, wherein said lipoxin comprises a lipoxin selected from the group consisting of 16-phenoxy-LXA4-Me, 15-cyclohexyl-LXA4-Me, and 15-R/S-methyl-LXA4-Me.

24. The drug delivery vehicle according to any one of claims 6-7, wherein said drug delivery vehicle comprises a TLR agonist shown in Table 1, and/or shown in FIG. 32, and/or selected from the group consisting of MEDI9197, 3M-052 (Telratolimod), MPLA (PHAD®), KRN7000, Kdo2-Lipid A ammonium, Pam2CSK4, Pam3CSK4, FSL-1, CRX-527, LXA4, Resolvins (D series 1-6), and Resolvins (E series 1-6).

25. The drug delivery vehicle according to any one of claims 1-24, wherein said chemotherapeutic agent is an ICD inducer selected from the group consisting of mitoxantrone (MTX), doxorubicin (DOX), oxaliplatin, anthracenedione, bleomycin, bortezomib, cisplatin, daunorubicin, docetaxel, epirubicin, idarubicin, paclitaxel, R2016, cyclophosphamide, and irinotecan.

26. The drug delivery vehicle of claim 25, wherein said chemotherapeutic agent comprises irinotecan (IRIN).

27. The drug delivery vehicle of claim 25, wherein said chemotherapeutic agent comprises mitoxantrone (MTX).

28. The drug delivery vehicle of claim 25, wherein said chemotherapeutic agent comprises oxaliplatin.

29. The drug delivery vehicle of claim 25, wherein said chemotherapeutic agent comprises doxorubicin.

30. The drug delivery vehicle of according to any one of claims 1-29, wherein said lipid bilayer comprises a phospholipid.

31. The drug delivery vehicle of claim 30, wherein said phospholipid comprises a saturated fatty acid with a C14-C20 carbon chain, and/or an unsaturated fatty acid with a C14-C20 carbon chain, and/or a natural lipid comprising a mixture of fatty acids with C12-C20 carbon chains.

32. The drug delivery vehicle of claim 31, wherein said phospholipid comprises a phospholipid selected from the group consisting of phosphatidylcholine (DPPC), 1,2-dimyristoleoyl-sn-glycero-3-phosphocholine (DMPC), 1,2-dioleoyl-sn-glycero-3-phosphocholine (DOPC), 1,2-distearoyl-sn-glycero-3-phospho-rac-glycerol (DSPG), 1,2-dipalmitoyl-sn-glycero-3-phosphoglycerol (DPPG), distearoylphosphatidylcholine (DSPC), 1,2-dieicosenoyl-sn-glycero-3-phosphocholine, and diactylphosphatidylcholine (DAPC).

33. The drug delivery vehicle of claim 31, wherein said phospholipid comprises a natural lipid selected from the group consisting of egg phosphatidylcholine (egg PC), and soy phosphatidylcholine (soy PC).

34. The drug delivery vehicle of claim 31, wherein said phospholipid comprises distearoylphosphatidylcholine (DSPC).

35. The drug delivery vehicle according to any one of claims 30-34, wherein said lipid bilayer comprises an mPEG phospholipid with a phospholipid C14-C18 carbon chain, and a PEG molecular weight ranging from about 350 Da to 5000 Da.

36. The drug delivery vehicle of claim 35, wherein said lipid bilayer comprises 1,2-distearoyl-sn-glycero-3-phosphoethanolamine-PEG (DSPE-PEG).

37. The drug delivery vehicle of claim 36, wherein said DSPE-PEG comprises DPSE-PEG_{2K} or DPSE-PEG_{5K}.

38. The drug delivery vehicle according to any one of claims 34-37, wherein said lipid bilayer comprises DSPC:CHOL and/or CHEMS:DSPE-PEG:lipid compatible TLR7/TLR8 agonist and/or lipoxin.

39. The drug delivery vehicle of claim 38, wherein the ratio of DSPC:CHOL and/or CHEMS:DSPE-PEG:TLR7/8 agonist ranges from 40-90% DSPC:10%-50% CHEL and/or CHEMS: 1%-10% DSPE-PEG:1%-20% TLR7/8 agonist (molar ratio).

40. The drug delivery vehicle of claim 39, wherein the lipid bilayer comprise 55.5:38.5:2.7:3.3 for DSPC, cholesterol, DSPE-PEG₂ₖ and TLR7/8 agonist.

41. The drug delivery vehicle of claim 40, wherein said TLR7/8 agonist comprises 3M-052.

42. The drug delivery vehicle of claim 38, wherein the ratio of DSPC:CHOL and/or CHEMS:DSPE-PEG:TLR7/8 agonist ranges from 40-90% DSPC:10%-50% CHEL and/or CHEMS: 1%-10% DSPE-PEG:0.1%-20% lipoxin (molar ratio).

43. The drug delivery vehicle of claim 42, wherein the lipid bilayer comprise 55.4:39.6:4.7:0.2 for DSPC, cholesterol, DSPE-PEG₂ₖ and lipoxin.

44. The drug delivery vehicle of claim 43, wherein said lipoxin comprises LXA4.

45. The drug delivery vehicle according to any one of claims 30-44, wherein said lipid bilayer comprises a cholesterol derivative selected from the group consisting of cholesterol hemisuccinate (CHEMS), lysine-based cholesterol (CHLYS), and PEGylated cholesterol (Chol-PEG).

46. The drug delivery vehicle of claim 45, wherein said lipid bilayer comprises CHEMS.

47. The drug delivery vehicle of claim 46, wherein said bilayer comprises CHEMS ranging from about 5% (mol percent) up to about 30% total lipid.

48. The drug delivery vehicle of claim 47, wherein said bilayer comprise about 10% or about 20% CHEMS or about 30% CHEMS or about 40% CHEMS.

49. The drug delivery vehicle according to any one of claims 1-48, wherein when the drug delivery vehicle contains a cargo-trapping agent (e.g., protonating agent).

50. The drug delivery vehicle of claim 49, wherein said cargo trapping agent before reaction with the chemotherapeutic agent loaded in drug delivery vehicle is selected from the group consisting of citric acid, triethylammonium sucrose octasulfate (TEA₈SOS), (NH₄)₂SO₄, an ammonium salt, a trimethylammonium salt, and a triethylammonium salt.

51. The drug delivery vehicle of claim 50, wherein said cargo-trapping agent before reaction with said chemotherapeutic agent is citric acid.

52. The drug delivery vehicle of claim 50, wherein said cargo-trapping agent before reaction with said chemotherapeutic agent is ammonium sulfate.

53. The drug delivery vehicle according to any one of claims 1-52, wherein said drug carrier is conjugated to a moiety selected from the group consisting of a targeting moiety, a fusogenic peptide, and a transport peptide.

54. The drug delivery vehicle according to any one of claims 1-53, wherein:
said drug delivery vehicle in suspension is stable for at least 1 month, or at least 2 months, or at least 3 months, or at least 4 months, or at least 5 months, or at least 6 months when stored at 4° C.; and/or
said drug delivery vehicle forms a stable suspension on rehydration after lyophilization; and/or
said drug delivery vehicle shows reduced drug toxicity as compared to free drug; and/or
said drug delivery vehicle colloidal stability in physiological fluids with pH 7.4 and remains monodisperse to allow systemic biodistribution and is capable of entering a disease site by vascular leakage (EPR effect) or transcytosis.

55. The drug delivery vehicle drug carrier according to any one of claims 1-54, wherein said carrier is colloidally stable.

56. A pharmaceutical formulation comprising:
a drug delivery vehicle according to any one of claims 1-55; and
a pharmaceutically acceptable carrier.

57. The pharmaceutical formulation of claim 56, wherein said formulation is formulated for administration via a route selected from the group consisting of intravenous administration, intraarterial administration, intracerebral administration, intrathecal administration, oral administration, aerosol administration, administration via inhalation, intracranial administration via a cannula, and subcutaneous or intramuscular depot deposition.

58. The pharmaceutical formulation according to any one of claims 56-57, wherein said formulation is formulated for systemic administration.

59. The pharmaceutical formulation according to any one of claims 56-58, wherein said formulation is sterile.

60. The pharmaceutical formulation according to any one of claims 56-59, wherein said formulation is a unit dosage formulation.

61. A method of treating a cancer in a mammal, said method comprising administering to said mammal an effective amount of a drug delivery carrier according to any one of claims 1-55.

62. The method of claim 61, wherein said administering comprises administering an effective amount of a pharmaceutical formation according to any one of claims 56-61.

63. The method according to any one of claims 61-62, wherein said cancer comprises a cancer selected from the group consisting of pancreatic ductal adenocarcinoma (PDAC). In certain embodiments the cancer is a cancer selected from the group consisting of acute lymphoblastic leukemia (ALL), acute myeloid leukemia (AML), adrenocortical carcinoma, AIDS-related cancers (e.g., Kaposi sarcoma, lymphoma), anal cancer, appendix cancer, astrocytomas, atypical teratoid/rhabdoid tumor, bile duct cancer, extrahepatic cancer, bladder cancer, bone cancer (e.g., Ewing sarcoma, osteosarcoma, malignant fibrous histiocytoma), brain stem glioma, brain tumors (e.g., astrocytomas, glioblastoma, brain and spinal cord tumors, brain stem glioma, central nervous system atypical teratoid/rhabdoid tumor, central nervous system embryonal tumors, central nervous system germ cell tumors, craniopharyngioma, ependymoma, breast cancer, bronchial tumors, burkitt lymphoma, carcinoid tumors (e.g., childhood, gastrointestinal), cardiac tumors, cervical cancer, chordoma, chronic lymphocytic leukemia (CLL), chronic myelogenous leukemia (CML), chronic myeloproliferative disorders, colon cancer, colorectal cancer, craniopharyngioma, cutaneous t-cell lymphoma, duct cancers e.g. (bile, extrahepatic), ductal carcinoma in situ (DCIS), embryonal tumors, endometrial cancer, ependymoma, esophageal cancer, esthesioneuroblastoma, extracranial germ cell tumor, extragonadal germ cell tumor, extrahepatic bile duct cancer, eye cancer (e.g., intraocular melanoma, retinoblastoma), fibrous histiocytoma of bone, malignant, and osteosarcoma, gallbladder cancer, gastric (stomach) cancer, gastrointestinal carcinoid tumor, gastrointestinal stromal tumors (GIST), germ cell tumors (e.g., ovarian cancer, testicular cancer, extracranial cancers, extragonadal cancers, central nervous system), gestational trophoblastic tumor, brain stem cancer, hairy cell leukemia, head and neck cancer, heart cancer, hepatocellular (liver) cancer, histiocytosis, langerhans cell cancer, Hodgkin lymphoma, hypopharyngeal cancer, intraocular melanoma, islet cell tumors, pancreatic neuroendocrine tumors, kaposi sarcoma, kidney cancer (e.g., renal cell, Wilm's tumor, and other kidney tumors), langerhans cell histiocytosis, laryngeal cancer, leukemia, acute lymphoblastic (ALL), acute myeloid (AML), chronic lymphocytic (CLL), chronic myelogenous (CML), hairy cell, lip and oral cavity cancer, liver cancer (primary), lobular carcinoma in situ (LCIS), lung cancer (e.g., childhood, non-small cell, small cell), lymphoma (e.g., AIDS-related, Burkitt (e.g., non-Hodgkin lymphoma), cutaneous T-Cell (e.g., mycosis fungoides, Sézary syndrome), Hodgkin, non-Hodgkin, primary central nervous system (CNS)), macroglobulinemia, Waldenström, male breast cancer, malignant fibrous histiocytoma of bone and osteosarcoma, melanoma (e.g., childhood, intraocular (eye)), merkel cell carcinoma, mesothelioma, metastatic squamous neck cancer, midline tract carcinoma, mouth cancer, multiple endocrine neoplasia syndromes, multiple myeloma/plasma cell neoplasm, mycosis fungoides, myelodysplastic syndromes, chronic myeloid leukemia (CML), multiple myeloma, nasal cavity and paranasal sinus cancer, nasopharyngeal cancer, neuroblastoma, oral cavity cancer, lip and oropharyngeal cancer, osteosarcoma, ovarian cancer, pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, plasma cell neoplasm, pleuropulmonary blastoma, primary central nervous system (CNS) lymphoma, prostate cancer, rectal cancer, renal cell (kidney) cancer, renal pelvis and ureter, transitional cell cancer, rhabdomyosarcoma, salivary gland cancer, sarcoma (e.g., Ewing, Kaposi, osteosarcoma, rhadomyosarcoma, soft tissue, uterine), Sézary syndrome, skin cancer (e.g., melanoma, merkel cell carcinoma, basal cell carcinoma, nonmelanoma), small intestine cancer, squamous cell carcinoma, squamous neck cancer with occult primary, stomach (gastric) cancer, testicular cancer, throat cancer, thymoma and thymic carcinoma, thyroid cancer, trophoblastic tumor, ureter and renal pelvis cancer, urethral cancer, uterine cancer, endometrial cancer, uterine sarcoma, vaginal cancer, vulvar cancer, Waldenström macroglobulinemia, and Wilm's tumor.

64. The method of claim 63, wherein said cancer comprise pancreatic cancer.

65. The method of claim 64, wherein said cancer comprises advanced PDAC.

66. The method according to any one of claims 64-65, wherein said drug delivery carrier comprises a component in a drug combination known as the FOLFIRINOX (folinic acid, 5-fluorouracil, irinotecan, and oxaliplatin) regimen.

67. The method of claim 66, wherein said drug delivery carrier comprises irinotecan and said carrier replaces irinotecan in the FOLFIRINOX drug combination.

68. The method of claim 66, wherein said drug delivery carrier comprises oxaliplatin and said carrier replaces oxaliplatin in the FOLFIRINOX drug combination.

69. The method of claim 66, wherein said drug delivery carrier is administered in addition to the combination of folinic acid, 5-fluorouracil, irinotecan, and oxaliplatin.
